SEQUENCE LISTING (51 sequences)

SEQ ID NO: 1 - RNA, 4262 nt, synthetic construct
  source 1..4262: mol_type = other RNA
gggaaataagagagaaaagaagagtaagaagaaatataagaccccggcgccgccaccatgttccttatcctgctgatcagcctgcccaccgccttcgccgtgatcggcgacctgaagtgcccactggagagccggaccggcagcctgaataacatcgataccggaccgcccagcatcagcaccgccaccgtggacgtgaccaacggcctgggcacctactacgtgctggaccgggtgtacctgaataccaccctgttcctgaacggctattaccccaccagcggcagcacctaccggaatatggcgctgaagggcaccgacaagctgagcaccctgtggttcaaaccacccttcctgagcgactttatcaacggcattttcgcgaaggtgaagaacaccaaggtgttcaaggacggcgtgatgtacagcgagttccccgccatcaccattgggagcaccttcgtcaacaccagctactctgtggtggtgcagccccggaccatcaacagcacccaggacggtgtgaacaagctgcagggcctgctggaggtgagcgtgtgccagtacaacatgtgcgagtatccccacaccatctgccaccccaacctgggcaaccacttcaaggagctgtggcacatggacaccggagtggtgagctgcctgtacaagcggaacttcacctacgacgtgaacgctacctacctgtacttccacttctaccaggagggcggcaccttctacgcctacttcaccgataccggcgtggtgaccaagttcctgttcaacgtgtacctgggcatggccctgagccactactacgttatgcccctgacctgtatcagccggcggaacatcggcttcaccctggagtactgggtgacccctctgaccagccggcagtacctgctggccttcaaccaggacggcatcatcttcaacgccgtggactgcatgtcagacttcatgagcgagatcaagtgcaagacccagagcatcgcccctcccaccggcgtgtacgagctgaacggttacaccgtgcagcccatcgccgacgtgtaccggcggaagcccgacctgcccaactgcaacatcgaggcctggctgaacgacaagagcgtgcccagcccactgaactgggagcggaagaccttcagtaactgcaacttcaacatgagcagcctgatgagcttcatccaggccgactcattcacctgcaacaacatcgacgccgccaagatctacggcatgtgcttcagcagcatcaccatcgacaagttcgccatcccaaacggccggaaggtggacttacagctgggcaatctgggctacctgcagagcttcaactaccggatcgacacgaccgccacctcctgccagttatactacaatctgcctgccgccaacgtgagcgtgagccgcttcaaccccagcacctggaacaagcggttcggcttcatcgaggacagcgtgttcaaaccccagcccgccggcgtgttcacaaaccacgacgtggtgtacgcccagcactgctttaaggcccctaagaacttctgcccctgcaagtccaatagcagcttgtgcgtcgggtctggccccggaaagaacaacgggattggcacctgccccgctggcaccaactacctgacctgccacaacctctgcaaccccgaccccatcaccttcaccggcccctacaagtgtccccagaccaagagcctggtgggcatcggcgagcactgcagcggcctggcagtgaagagcgactactgcggcggcaacctgtgcacctgccagccccaggccttcctgggctggagcgccgacagctgcctgcagggcgacaagtgcaacatcttcgccaatctgatcctgcacgacgtcaacagcggactgacctgcagcaccgacctgcagaaggccaacaccgacatcaagctgggcgtgtgcgtgaactacgacctgtacggcatcagcggccagggcatcttcgtggaggtcaacgccacctattacaacagctggcagaacctgctgtacgacagcaacggtaatttatacggcttccgggactacatcaccaaccggaccttcatgatccggagctgctacagcggccgggtgagcgcagctttccacgccaacagcagcgagcccgccctgctgtttcggaacatcaagtgtaactacgtgttcaacaacagcctgacccggcagctgcagcctatcaactacttcgacagctacctgggctgcgtggtgaacgcatacaacagcactgccatcagcgtgcagacctgcgacctgaccgtgggctccgggtactgcgtggactacagcaagaacggcggctccggcggagctatcaccaccggctaccggttcaccaacttcgagcccttcaccgtgaacagcgtgaacgacagcctggagcccgtgggcggactgtacgagatccagatccccagcgacttcaccatcggcaacatggaggagttcatccagacctcaagccccaaggtgaccatcgactgcgccgccttcgtgtgcggcgactacgccgcctgcaagagccagctggtggagtacggcagcttctgcgacaacatcaacgccatcctgaccgaggtgaacgaactgctggacaccacccagttacaggtggccaacagtctgatgaacggcgtgaccctgagtatcaagctgaaggacggagtgaacttcaacgtggacgacatcaatttcagcagcgtgctgggctgcctgggcagcgagtgcagcaaggccagtagccggagcgccatagaggacctgctgttcgacaaggtgaaactgagcgacgtgggcttcgtggccgcctacaacaactgcaccggtggcgccgagatccgggacctgatctgcgtgcagagctacaagggcatcaaagtgctgccacctctgctgagcgagaaccagatcagcggctacaccctcgccgccaccagcgctagcctgtttcctccttggactgctgccgcaggcgtgcccttctacctgaacgtgcagtaccggattaacggcctcggtgtgaccatggacgtgctgagccagaaccagaagctgatcgccaacgccttcaacaacgccctggacgccatccaggagggattcgacgccaccaacagcgccctggtgaagatccaggcggtagtgaacgccaacgctgaggccctgaacaacctgctgcaacagctgagcaaccggttcggcgccatcagcagcagcctgcaggagatcctgagccggctggatccaccagaggccgaggcccagatcgaccggctgatcaacggccggctgaccgccctgaacgcctacgtgagccagcagctgagcgacagcaccctggtcaagttcagcgccgcccaggccatggagaaagtgaacgagtgcgtgaagagccagagcagccggatcaacttctgcggcaacggcaaccacatcatcagcctggtgcagaacgccccttacggcctgtactttatccacttcagctacgtgcccaccaagtacgtgaccgccaaggtgagccccggcctgtgcatcgccggcgaccggggaatcgctcccaagagcggctacttcgtgaacgtgaacaacacctggatgtacaccggcagcggatactactaccccgagcccatcaccgagaacaacgtggtggtgatgagcacctgcgcagtgaactacaccaaggctccttacgtgatgctgaacacctctatcccaaatctgcccgacttccgggaggagctggaccagtggttcaagaaccagaccagcgtggcccctgatctgagcctggactacattaacgtgaccttcctggacctgcaggtggagatgaaccggctgcaggaggccatcaaggtgctgaaccagtcatacatcaacctgaaggacatcggcacctacgagtactacgtgaagtggccctggtacgtgtggctgctgatctgcctggccggcgtggccatgctggtgctgctgttcttcatctgctgctgcaccggctgcggcaccagctgcttcaagaagtgcggcggctgctgcgacgactacaccggataccaggagctggtgatcaagaccagccacgacgactgataataggctggagcctcggtggcctagcttcttgccccttgggcctccccccagcccctcctccccttcctgcacccgtacccccgtggtctttgaataaagtctgagtgggcggc

SEQ ID NO: 2 - RNA, 57 nt, synthetic construct
  source 1..57: mol_type = other RNA
gggaaataagagagaaaagaagagtaagaagaaatataagaccccggcgccgccacc

SEQ ID NO: 3 - RNA, 4086 nt, synthetic construct
  source 1..4086: mol_type = other RNA
atgttccttatcctgctgatcagcctgcccaccgccttcgccgtgatcggcgacctgaagtgcccactggagagccggaccggcagcctgaataacatcgataccggaccgcccagcatcagcaccgccaccgtggacgtgaccaacggcctgggcacctactacgtgctggaccgggtgtacctgaataccaccctgttcctgaacggctattaccccaccagcggcagcacctaccggaatatggcgctgaagggcaccgacaagctgagcaccctgtggttcaaaccacccttcctgagcgactttatcaacggcattttcgcgaaggtgaagaacaccaaggtgttcaaggacggcgtgatgtacagcgagttccccgccatcaccattgggagcaccttcgtcaacaccagctactctgtggtggtgcagccccggaccatcaacagcacccaggacggtgtgaacaagctgcagggcctgctggaggtgagcgtgtgccagtacaacatgtgcgagtatccccacaccatctgccaccccaacctgggcaaccacttcaaggagctgtggcacatggacaccggagtggtgagctgcctgtacaagcggaacttcacctacgacgtgaacgctacctacctgtacttccacttctaccaggagggcggcaccttctacgcctacttcaccgataccggcgtggtgaccaagttcctgttcaacgtgtacctgggcatggccctgagccactactacgttatgcccctgacctgtatcagccggcggaacatcggcttcaccctggagtactgggtgacccctctgaccagccggcagtacctgctggccttcaaccaggacggcatcatcttcaacgccgtggactgcatgtcagacttcatgagcgagatcaagtgcaagacccagagcatcgcccctcccaccggcgtgtacgagctgaacggttacaccgtgcagcccatcgccgacgtgtaccggcggaagcccgacctgcccaactgcaacatcgaggcctggctgaacgacaagagcgtgcccagcccactgaactgggagcggaagaccttcagtaactgcaacttcaacatgagcagcctgatgagcttcatccaggccgactcattcacctgcaacaacatcgacgccgccaagatctacggcatgtgcttcagcagcatcaccatcgacaagttcgccatcccaaacggccggaaggtggacttacagctgggcaatctgggctacctgcagagcttcaactaccggatcgacacgaccgccacctcctgccagttatactacaatctgcctgccgccaacgtgagcgtgagccgcttcaaccccagcacctggaacaagcggttcggcttcatcgaggacagcgtgttcaaaccccagcccgccggcgtgttcacaaaccacgacgtggtgtacgcccagcactgctttaaggcccctaagaacttctgcccctgcaagtccaatagcagcttgtgcgtcgggtctggccccggaaagaacaacgggattggcacctgccccgctggcaccaactacctgacctgccacaacctctgcaaccccgaccccatcaccttcaccggcccctacaagtgtccccagaccaagagcctggtgggcatcggcgagcactgcagcggcctggcagtgaagagcgactactgcggcggcaacctgtgcacctgccagccccaggccttcctgggctggagcgccgacagctgcctgcagggcgacaagtgcaacatcttcgccaatctgatcctgcacgacgtcaacagcggactgacctgcagcaccgacctgcagaaggccaacaccgacatcaagctgggcgtgtgcgtgaactacgacctgtacggcatcagcggccagggcatcttcgtggaggtcaacgccacctattacaacagctggcagaacctgctgtacgacagcaacggtaatttatacggcttccgggactacatcaccaaccggaccttcatgatccggagctgctacagcggccgggtgagcgcagctttccacgccaacagcagcgagcccgccctgctgtttcggaacatcaagtgtaactacgtgttcaacaacagcctgacccggcagctgcagcctatcaactacttcgacagctacctgggctgcgtggtgaacgcatacaacagcactgccatcagcgtgcagacctgcgacctgaccgtgggctccgggtactgcgtggactacagcaagaacggcggctccggcggagctatcaccaccggctaccggttcaccaacttcgagcccttcaccgtgaacagcgtgaacgacagcctggagcccgtgggcggactgtacgagatccagatccccagcgacttcaccatcggcaacatggaggagttcatccagacctcaagccccaaggtgaccatcgactgcgccgccttcgtgtgcggcgactacgccgcctgcaagagccagctggtggagtacggcagcttctgcgacaacatcaacgccatcctgaccgaggtgaacgaactgctggacaccacccagttacaggtggccaacagtctgatgaacggcgtgaccctgagtatcaagctgaaggacggagtgaacttcaacgtggacgacatcaatttcagcagcgtgctgggctgcctgggcagcgagtgcagcaaggccagtagccggagcgccatagaggacctgctgttcgacaaggtgaaactgagcgacgtgggcttcgtggccgcctacaacaactgcaccggtggcgccgagatccgggacctgatctgcgtgcagagctacaagggcatcaaagtgctgccacctctgctgagcgagaaccagatcagcggctacaccctcgccgccaccagcgctagcctgtttcctccttggactgctgccgcaggcgtgcccttctacctgaacgtgcagtaccggattaacggcctcggtgtgaccatggacgtgctgagccagaaccagaagctgatcgccaacgccttcaacaacgccctggacgccatccaggagggattcgacgccaccaacagcgccctggtgaagatccaggcggtagtgaacgccaacgctgaggccctgaacaacctgctgcaacagctgagcaaccggttcggcgccatcagcagcagcctgcaggagatcctgagccggctggatccaccagaggccgaggcccagatcgaccggctgatcaacggccggctgaccgccctgaacgcctacgtgagccagcagctgagcgacagcaccctggtcaagttcagcgccgcccaggccatggagaaagtgaacgagtgcgtgaagagccagagcagccggatcaacttctgcggcaacggcaaccacatcatcagcctggtgcagaacgccccttacggcctgtactttatccacttcagctacgtgcccaccaagtacgtgaccgccaaggtgagccccggcctgtgcatcgccggcgaccggggaatcgctcccaagagcggctacttcgtgaacgtgaacaacacctggatgtacaccggcagcggatactactaccccgagcccatcaccgagaacaacgtggtggtgatgagcacctgcgcagtgaactacaccaaggctccttacgtgatgctgaacacctctatcccaaatctgcccgacttccgggaggagctggaccagtggttcaagaaccagaccagcgtggcccctgatctgagcctggactacattaacgtgaccttcctggacctgcaggtggagatgaaccggctgcaggaggccatcaaggtgctgaaccagtcatacatcaacctgaaggacatcggcacctacgagtactacgtgaagtggccctggtacgtgtggctgctgatctgcctggccggcgtggccatgctggtgctgctgttcttcatctgctgctgcaccggctgcggcaccagctgcttcaagaagtgcggcggctgctgcgacgactacaccggataccaggagctggtgatcaagaccagccacgacgac

SEQ ID NO: 4 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - protein, 1362 aa, synthetic construct
MFLILLISLPTAFAVIGDLKCPLESRTGSLNNIDTGPPSISTATVDVTNGLGTYYVLDRVYLNTTLFLNGYYPTSGSTYRNMALKGTDKLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHMDTGVVSCLYKRNFTYDVNATYLYFHFYQEGGTFYAYFTDTGVVTKFLFNVYLGMALSHYYVMPLTCISRRNIGFTLEYWVTPLTSRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPQPAGVFTNHDVVYAQHCFKAPKNFCPCKSNSSLCVGSGPGKNNGIGTCPAGTNYLTCHNLCNPDPITFTGPYKCPQTKSLVGIGEHCSGLAVKSDYCGGNLCTCQPQAFLGWSADSCLQGDKCNIFANLILHDVNSGLTCSTDLQKANTDIKLGVCVNYDLYGISGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNGGSGGAITTGYRFTNFEPFTVNSVNDSLEPVGGLYEIQIPSDFTIGNMEEFIQTSSPKVTIDCAAFVCGDYAACKSQLVEYGSFCDNINAILTEVNELLDTTQLQVANSLMNGVTLSIKLKDGVNFNVDDINFSSVLGCLGSECSKASSRSAIEDLLFDKVKLSDVGFVAAYNNCTGGAEIRDLICVQSYKGIKVLPPLLSENQISGYTLAATSASLFPPWTAAAGVPFYLNVQYRINGLGVTMDVLSQNQKLIANAFNNALDAIQEGFDATNSALVKIQAVVNANAEALNNLLQQLSNRFGAISSSLQEILSRLDPPEAEAQIDRLINGRLTALNAYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMYTGSGYYYPEPITENNVVVMSTCAVNYTKAPYVMLNTSIPNLPDFREELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 6 - RNA, 4244 nt, synthetic construct
  source 1..4244: mol_type = other RNA
gggaaataagagagaaaagaagagtaagaagaaatataagaccccggcgccgccaccatgctgctgatcatctttatcctgcccaccaccctggccgtgatcggagacttcaactgtaccaacttcgctattaacgacctgaacacaaccataccccggatcagcgagtacgtggtggacgtgagctacggtctgggcacctactacatcctggaccgggtgtacctgaataccaccatcctgtttaccggctacttccccaagagcggcgccaacttccgggacctgagcctgaagggcaccaccaagctgagcaccctgtggtaccagaagcccttcctctccgacttcaataacggcatcttcagccgggtgaagaataccaagctatacgtgaacaagaccctgtacagcgagttcagcaccatagtgatcggcagcgtgtttatcaacaatagctatacaatcgtggtgcagccccacaacggcgtgctggagatcacagcctgccagtacaccatgtgcgagtatccccacaccatctgcaagtcaattggcagctcccggaacgagagctggcacttcgataagagcgagcccctgtgcctattcaagaagaactttacttacaacgtgagcaccgattggctgtacttccacttctaccaggagcggggcaccttctacgcctactacgcagacagcggcatgcccactaccttcctgttcagcttatacctgggaaccctgctaagccactattacgtgctgcccctgacctgcaacgccatctcgagcaacaccgacaacgagaccctgcagtactgggtgacccctctgagcaagcggcagtacctgctgaaattcgacaaccggggcgtgatcaccaacgccgtggactgcagtagcagcttctttagcgagatccagtgcaagaccaagagcctgctgcccaacaccggcgtgtacgatctgagcggatttaccgtgaagcccgtggccaccgtgcaccggcggatccccgacctgcctgactgcgacatcgacaagtggctgaataactttaacgtgcccagtcctctgaactgggagcggaagattttcagtaactgcaactttaatctgtccaccctgctgcggctggtgcacaccgatagctttagctgcaacaacttcgacgagagcaagatctacggcagctgcttcaagagcatcgtgctcgataagttcgccatccccaacagccggcggagcgacctgcagctgggcagtagcggattcctgcagagcagcaattacaagatcgacaccaccagcagctcttgccagttgtactacagcctacccgccatcaacgtgacaatcaacaactacaaccctagcagctggaaccggcggtacggctttaacaattttaacctgagcagtcacagtgtggtgtacagccggtactgcttcagtgtgaacaacaccttttgcccctgcgccaagcccagcttcgccagctcgtgcaagagccacaagccacctagcgccagctgccccatcggcaccaactaccggagctgcgagagcaccacagtgctggaccacaccgactggtgccggtgtagctgcctgcccgaccccatcaccgcctacgaccctcggagctgtagccagaagaagtctctggtgggcgtgggcgagcactgcgccggcttcggcgtggacgaggagaagtgcggcgtactggacgggagctataacgtgagttgcctgtgtagcaccgacgccttcctggggtggagctacgacacctgcgtgagcaacaaccggtgcaacatattcagcaatttcatcctgaacggaattaactctggcaccacttgcagtaacgatctgctgcagcccaatacggaggtgtataccgacgtgtgcgttgactacgacttgtacggcatcaccggacagggcatctttaaggaggtgtcggccgtgtactataactcttggcagaacctgctctacgactccaacggcaacatcatcggtttcaaggactttgtgaccaacaagacatacaacatctttccctgctacgctggtcgggtgagcgccgcgttccaccagaacgccagcagcctggctctgctgtaccggaacctcaagtgcagctacgtgctcaacaacattagcctggccacccagccctacttcgacagctacctgggctgcgtgttcaacgccgacaacctgaccgactacagcgtgtcaagctgcgccctgcggatgggcagcggcttctgcgtggactataacagccccagcttttccagcagcggcggctccggtggtagcatctccgccagctaccggttcgtgaccttcgagcccttcaacgtcagcttcgtgaacgacagcatcgagagtgtgggcggtctgtacgagatcaagatccccaccaactttaccatcgtgggccaggaggagttcatccagaccaacagtcccaaggtgaccatcgactgcagcctgttcgtgtgcagcaactacgccgcctgccacgacctgttatcggagtacggtaccttctgcgacaatatcaacagcatcttagacgaagtgaacggcctgctggacaccactcagctgcacgtggctgacaccctgatgcaaggcgtgactctgtctagcaaccttaacaccaacctgcatttcgacgtggacaacatcaatttcaagtccctggtggggtgcctggggcctcactgcggctcaagcagccggagcttcttcgaggacctgttgttcgacaaggtgaagctgtccgacgtgggcttcgtggaggcctacaacaactgcactggtggctccgagatccgggacttactgtgcgtgcagagcttcaacggcattaaggtgctgcctcccatcctgagcgagagccaaatcagcggctacaccaccgccgccacggttgctgccatgtttccgccttggagcgctgcggccgggatacccttcagcctgaacgtgcagtacagaatcaacggactgggagtgaccatggacgtgttgaataagaatcagaagctgatcgccaccgccttcaacaacgccctgctgagcatccagaacggcttcagcgccaccaacagcgccctggccaagatccagagcgtggtgaacagcaacgcccaggccctgaatagcctgctacagcagctgttcaacaagttcggggccatctcttccagcctgcaggagatcctgtctcggctggaccctcctgaggcccaggtgcagatcgaccggctgatcaacggccggctgaccgccctgaacgcctacgtgagccagcaactgagcgatatcagcctggtgaagttcggtgccgctctggccatggagaaggtgaacgagtgcgtgaagagccagagcccacggatcaacttctgcggaaacggtaaccacatcctgtcactggtgcagaacgctccctacggccttctgttcatgcacttcagctacaagcccatctcattcaagaccgtgctggtgagccccggcctgtgcatcagcggggacgtgggaatcgcgcccaagcagggctactttatcaagcacaacgaccactggatgttcaccggcagcagctactactaccccgagcccatcagcgacaagaacgtggtgttcatgaatacttgcagcgtgaatttcaccaaggccccgctggtgtatctgaaccacagcgtgcccaagctgtcagacttcgaaagcgagctgagccactggttcaagaaccagaccagcatcgccccaaacctgacactgaacctgcacaccattaacgccacctttctggacctgtactacgaaatgaacctgatccaggagagcatcaagagcttaaacaacagctacatcaacctgaaggatatcggcacatacgagatgtacgtgaagtggccctggtacgtgtggctgctcataagcttcagcttcatcatcttcctggtgctgctgttcttcatctgctgttgcacgggctgcggcagcgcttgcttcagcaagtgccacaactgctgcgacgagtacggcggccaccacgacttcgtgatcaagaccagccacgacgactgataataggctggagcctcggtggcctagcttcttgccccttgggcctccccccagcccctcctccccttcctgcacccgtacccccgtggtctttgaataaagtctgagtgggcggc

SEQ ID NO: 7 - RNA, 4068 nt, synthetic construct
  source 1..4068: mol_type = other RNA
atgctgctgatcatctttatcctgcccaccaccctggccgtgatcggagacttcaactgtaccaacttcgctattaacgacctgaacacaaccataccccggatcagcgagtacgtggtggacgtgagctacggtctgggcacctactacatcctggaccgggtgtacctgaataccaccatcctgtttaccggctacttccccaagagcggcgccaacttccgggacctgagcctgaagggcaccaccaagctgagcaccctgtggtaccagaagcccttcctctccgacttcaataacggcatcttcagccgggtgaagaataccaagctatacgtgaacaagaccctgtacagcgagttcagcaccatagtgatcggcagcgtgtttatcaacaatagctatacaatcgtggtgcagccccacaacggcgtgctggagatcacagcctgccagtacaccatgtgcgagtatccccacaccatctgcaagtcaattggcagctcccggaacgagagctggcacttcgataagagcgagcccctgtgcctattcaagaagaactttacttacaacgtgagcaccgattggctgtacttccacttctaccaggagcggggcaccttctacgcctactacgcagacagcggcatgcccactaccttcctgttcagcttatacctgggaaccctgctaagccactattacgtgctgcccctgacctgcaacgccatctcgagcaacaccgacaacgagaccctgcagtactgggtgacccctctgagcaagcggcagtacctgctgaaattcgacaaccggggcgtgatcaccaacgccgtggactgcagtagcagcttctttagcgagatccagtgcaagaccaagagcctgctgcccaacaccggcgtgtacgatctgagcggatttaccgtgaagcccgtggccaccgtgcaccggcggatccccgacctgcctgactgcgacatcgacaagtggctgaataactttaacgtgcccagtcctctgaactgggagcggaagattttcagtaactgcaactttaatctgtccaccctgctgcggctggtgcacaccgatagctttagctgcaacaacttcgacgagagcaagatctacggcagctgcttcaagagcatcgtgctcgataagttcgccatccccaacagccggcggagcgacctgcagctgggcagtagcggattcctgcagagcagcaattacaagatcgacaccaccagcagctcttgccagttgtactacagcctacccgccatcaacgtgacaatcaacaactacaaccctagcagctggaaccggcggtacggctttaacaattttaacctgagcagtcacagtgtggtgtacagccggtactgcttcagtgtgaacaacaccttttgcccctgcgccaagcccagcttcgccagctcgtgcaagagccacaagccacctagcgccagctgccccatcggcaccaactaccggagctgcgagagcaccacagtgctggaccacaccgactggtgccggtgtagctgcctgcccgaccccatcaccgcctacgaccctcggagctgtagccagaagaagtctctggtgggcgtgggcgagcactgcgccggcttcggcgtggacgaggagaagtgcggcgtactggacgggagctataacgtgagttgcctgtgtagcaccgacgccttcctggggtggagctacgacacctgcgtgagcaacaaccggtgcaacatattcagcaatttcatcctgaacggaattaactctggcaccacttgcagtaacgatctgctgcagcccaatacggaggtgtataccgacgtgtgcgttgactacgacttgtacggcatcaccggacagggcatctttaaggaggtgtcggccgtgtactataactcttggcagaacctgctctacgactccaacggcaacatcatcggtttcaaggactttgtgaccaacaagacatacaacatctttccctgctacgctggtcgggtgagcgccgcgttccaccagaacgccagcagcctggctctgctgtaccggaacctcaagtgcagctacgtgctcaacaacattagcctggccacccagccctacttcgacagctacctgggctgcgtgttcaacgccgacaacctgaccgactacagcgtgtcaagctgcgccctgcggatgggcagcggcttctgcgtggactataacagccccagcttttccagcagcggcggctccggtggtagcatctccgccagctaccggttcgtgaccttcgagcccttcaacgtcagcttcgtgaacgacagcatcgagagtgtgggcggtctgtacgagatcaagatccccaccaactttaccatcgtgggccaggaggagttcatccagaccaacagtcccaaggtgaccatcgactgcagcctgttcgtgtgcagcaactacgccgcctgccacgacctgttatcggagtacggtaccttctgcgacaatatcaacagcatcttagacgaagtgaacggcctgctggacaccactcagctgcacgtggctgacaccctgatgcaaggcgtgactctgtctagcaaccttaacaccaacctgcatttcgacgtggacaacatcaatttcaagtccctggtggggtgcctggggcctcactgcggctcaagcagccggagcttcttcgaggacctgttgttcgacaaggtgaagctgtccgacgtgggcttcgtggaggcctacaacaactgcactggtggctccgagatccgggacttactgtgcgtgcagagcttcaacggcattaaggtgctgcctcccatcctgagcgagagccaaatcagcggctacaccaccgccgccacggttgctgccatgtttccgccttggagcgctgcggccgggatacccttcagcctgaacgtgcagtacagaatcaacggactgggagtgaccatggacgtgttgaataagaatcagaagctgatcgccaccgccttcaacaacgccctgctgagcatccagaacggcttcagcgccaccaacagcgccctggccaagatccagagcgtggtgaacagcaacgcccaggccctgaatagcctgctacagcagctgttcaacaagttcggggccatctcttccagcctgcaggagatcctgtctcggctggaccctcctgaggcccaggtgcagatcgaccggctgatcaacggccggctgaccgccctgaacgcctacgtgagccagcaactgagcgatatcagcctggtgaagttcggtgccgctctggccatggagaaggtgaacgagtgcgtgaagagccagagcccacggatcaacttctgcggaaacggtaaccacatcctgtcactggtgcagaacgctccctacggccttctgttcatgcacttcagctacaagcccatctcattcaagaccgtgctggtgagccccggcctgtgcatcagcggggacgtgggaatcgcgcccaagcagggctactttatcaagcacaacgaccactggatgttcaccggcagcagctactactaccccgagcccatcagcgacaagaacgtggtgttcatgaatacttgcagcgtgaatttcaccaaggccccgctggtgtatctgaaccacagcgtgcccaagctgtcagacttcgaaagcgagctgagccactggttcaagaaccagaccagcatcgccccaaacctgacactgaacctgcacaccattaacgccacctttctggacctgtactacgaaatgaacctgatccaggagagcatcaagagcttaaacaacagctacatcaacctgaaggatatcggcacatacgagatgtacgtgaagtggccctggtacgtgtggctgctcataagcttcagcttcatcatcttcctggtgctgctgttcttcatctgctgttgcacgggctgcggcagcgcttgcttcagcaagtgccacaactgctgcgacgagtacggcggccaccacgacttcgtgatcaagaccagccacgacgac

SEQ ID NO: 8 - protein, 1356 aa, synthetic construct
MLLIIFILPTTLAVIGDFNCTNFAINDLNTTIPRISEYVVDVSYGLGTYYILDRVYLNTTILFTGYFPKSGANFRDLSLKGTTKLSTLWYQKPFLSDFNNGIFSRVKNTKLYVNKTLYSEFSTIVIGSVFINNSYTIVVQPHNGVLEITACQYTMCEYPHTICKSIGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVYTDVCVDYDLYGITGQGIFKEVSAVYYNSWQNLLYDSNGNIIGFKDFVTNKTYNIFPCYAGRVSAAFHQNASSLALLYRNLKCSYVLNNISLATQPYFDSYLGCVFNADNLTDYSVSSCALRMGSGFCVDYNSPSFSSSGGSGGSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDPPEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNNSYINLKDIGTYEMYVKWPWYVWLLISFSFIIFLVLLFFICCCTGCGSACFSKCHNCCDEYGGHHDFVIKTSHDD

SEQ ID NO: 9 - RNA, 3689 nt, synthetic construct
  source 1..3689: mol_type = other RNA
gggaaataagagagaaaagaagagtaagaagaaatataagaccccggcgccgccaccatgttcgtgctgctggtggcctacgccctgctgcacatcgccggctgccagaccattaacggcacaaacaccagccactccgtttgcaacggctgcgtgggccacagcgagaacgtcttcgccgtggagagcggcggctacatccctagcaacttcgccttcaacaactggttcctgctgaccaacaccagctccgtggtggacggcgtggtgcggagcttccagcccctgctgctgaactgcctgtggagtgtgagcggcagccagttcatcaccggcttcgtgtacttcaacggcaccggcaagggcgcctgcaagggcttctacagcaacgccagctctgacgtgattcggtacaacataaacttcgaggagaacctgcggcggggcaccatcctgttcaagaccagctacggcgccgtggtgttctactgcaccaacaacaccctggtgagcggcgacgcccacatccccagcggcaccgtgctgggcaacttctactgcttcgtgaacaccaccatcggcaacgagacgaccagcgccttcgtgggcgccctgcccaagaccgtgcgggagttcgtgatcagccggaccggccacttctacattaacggataccggtacttctccctgggcaacgtggaggccgtgaactttaacgtgaccaacgccgccaccaccgtgtgcaccgtagccctggccagctacgccgacgtgctcgtaaacgtgagccagaccgccatcgccaacatcatctactgcaacagcgtgatcaatcggctgcggtgcgaccagctgagcttcgacgtgcccgacggcttctactctaccagccccatccagcctgtggaactgcccgtgagcatcgtgagcctgcccgtgtaccacaagcacaccttcatcgtgctgtacgtgaacttcgagcaccagagaggccccggcaagtgctacaactgccggcccgccgtgatcaacatcaccctggctaacttcaacgagaccaagggccctctgtgcgttgataccagccatttcaccacccagttcgtggacaacgtcaagctggcccggtggagcgccagcatcgacaccggcaactgccccttcagcttcggcaaggtgatcaacttcgtgaagttcggcagcgtgtgcttcagcctgaagtacatcccaggcggctgcgccatgcccatcatggccaacctggtgaactacaagagccacaacatcggcagcttatacgtgagctggagcgacggcgacgtgatcaccggcgtgcccaagcccgtggagggcgtgtctagcttcatgaacgtgaccctgaacaagtgcactaagtacaacatctacgacgtcagcggcgtgggcgtgatacggatcagcaacgacaccttcctgaacggcatcacctacaccagcaccagcggcaacctgctgggcttcaaggacgtgacgaacggcaccatctacagcatcaccccttgcaaccctcctgaccagctggtggtgtaccagcaggccgtggtgggcgccatgttaagcgagaacttcacctcttacggcttcagcaacgtggtggagatgcccaagttcttctacgccagcaacggcacttacaactgcaccgacgccgtgctgacctactcaagcttcggcgtgtgcgccgacggcagcatcatcgccgtgcagccccggaacgtgagctacgacagcgtgagcgccatcgtgaccgccaacctgtctatccccagcaactggaccaccagcgtgcaggtggagtacctgcagattaccagcactcccatcgtggtggattgcagcacctacgtgtgcaacggcaacgtgcggtgcgtggagctgctgaagcagtacaccagcgcttgcaagaccatcgaggacgccctgcggaacagcgccatgctggagtctgccgacgtgagcgagatgctgaccttcgacaagaaggccttcacactggccaacgtgagcagctttggcgactacaacctgagcagcgtgattccaagccttcctcggagcggctctagggtggccggccggtctgcaatcgaggacatcttgttcagcaagctggtgaccagcggcctgggcacagtggacgccgactacaagaagtgcaccaaaggcctgagcatcgccgatctggcctgcgcccagtactacaacggaatcatggtgcttccaggggctgccgacgcagagcggatggccatgtacaccggcagcctgatcggcggaatcgccctgggcggactgaccagcgctgccagcatcccctttagcctggccatccagagccggctgaactacgtggcccttcagactgacgtgctgcaggagaaccagcggatcctggccgccagcttcaacaaggccatgaccaacatcgtggacgccttcacaggcgtgaacgacgccatcacccagaccagccaggccctgcagaccgtggccaccgccctcaacaagatccaggacgtggtgaaccagcagggcaacagcctgaaccacctgacctctcagctgcggcagaacttccaggccatcagcagtagcatccaagcaatctacgaccggctggatccaccacaggccgaccagcaggtggaccggctgataaccggccgcctggctgcccttaacgtgttcgtgagccataccctgaccaagtacaccgaggtgcgggcctctcggcagctggcccagcagaaggtgaacgagtgcgtgaagagccagagcaagcggtacggcttctgcggcaacgggacccacatcttcagccttgtgaacgccgcccctgaaggcctggtgttcctgcacaccgtgctactgcccacccagtacaaggacgttgaggcctggagcggcctgtgcgtggacgggatcaacggctacgtgctccggcagcccaacctggccctgtacaaggagggcaactactaccggatcaccagccggatcatgttcgagccccggatccccaccatcgccgacttcgtgcagatcgagaactgcaacgtgacctttgtgaacattagccggagcgagctgcagaccatcgtgcccgagtacatcgacgtgaacaagaccctgcaggagctgagctacaagctgcccaactacaccgttcccgacctggtggtggagcagtacaaccagaccatcctgaacctgacaagcgagataagcaccctggagaacaagagcgccgagctgaattacaccgtgcagaagctccagacactgatcgacaacatcaacagcactctggtggacctgaagtggctgaaccgggtggagacctacatcaagtggccctggtgggtgtggctgtgcatcagcgtggtgctgatcttcgtggtgagcatgctgctgctgtgctgctgcagcaccggctgctgcggcttcttcagctgcttcgccagcagcatccggggttgctgcgagagcaccaagttaccctactacgacgtggagaagatccacatccagtgataataggctggagcctcggtggcctagcttcttgccccttgggcctccccccagcccctcctccccttcctgcacccgtacccccgtggtctttgaataaagtctgagtgggcggc

SEQ ID NO: 10 - RNA, 3513 nt, synthetic construct
  source 1..3513: mol_type = other RNA
atgttcgtgctgctggtggcctacgccctgctgcacatcgccggctgccagaccattaacggcacaaacaccagccactccgtttgcaacggctgcgtgggccacagcgagaacgtcttcgccgtggagagcggcggctacatccctagcaacttcgccttcaacaactggttcctgctgaccaacaccagctccgtggtggacggcgtggtgcggagcttccagcccctgctgctgaactgcctgtggagtgtgagcggcagccagttcatcaccggcttcgtgtacttcaacggcaccggcaagggcgcctgcaagggcttctacagcaacgccagctctgacgtgattcggtacaacataaacttcgaggagaacctgcggcggggcaccatcctgttcaagaccagctacggcgccgtggtgttctactgcaccaacaacaccctggtgagcggcgacgcccacatccccagcggcaccgtgctgggcaacttctactgcttcgtgaacaccaccatcggcaacgagacgaccagcgccttcgtgggcgccctgcccaagaccgtgcgggagttcgtgatcagccggaccggccacttctacattaacggataccggtacttctccctgggcaacgtggaggccgtgaactttaacgtgaccaacgccgccaccaccgtgtgcaccgtagccctggccagctacgccgacgtgctcgtaaacgtgagccagaccgccatcgccaacatcatctactgcaacagcgtgatcaatcggctgcggtgcgaccagctgagcttcgacgtgcccgacggcttctactctaccagccccatccagcctgtggaactgcccgtgagcatcgtgagcctgcccgtgtaccacaagcacaccttcatcgtgctgtacgtgaacttcgagcaccagagaggccccggcaagtgctacaactgccggcccgccgtgatcaacatcaccctggctaacttcaacgagaccaagggccctctgtgcgttgataccagccatttcaccacccagttcgtggacaacgtcaagctggcccggtggagcgccagcatcgacaccggcaactgccccttcagcttcggcaaggtgatcaacttcgtgaagttcggcagcgtgtgcttcagcctgaagtacatcccaggcggctgcgccatgcccatcatggccaacctggtgaactacaagagccacaacatcggcagcttatacgtgagctggagcgacggcgacgtgatcaccggcgtgcccaagcccgtggagggcgtgtctagcttcatgaacgtgaccctgaacaagtgcactaagtacaacatctacgacgtcagcggcgtgggcgtgatacggatcagcaacgacaccttcctgaacggcatcacctacaccagcaccagcggcaacctgctgggcttcaaggacgtgacgaacggcaccatctacagcatcaccccttgcaaccctcctgaccagctggtggtgtaccagcaggccgtggtgggcgccatgttaagcgagaacttcacctcttacggcttcagcaacgtggtggagatgcccaagttcttctacgccagcaacggcacttacaactgcaccgacgccgtgctgacctactcaagcttcggcgtgtgcgccgacggcagcatcatcgccgtgcagccccggaacgtgagctacgacagcgtgagcgccatcgtgaccgccaacctgtctatccccagcaactggaccaccagcgtgcaggtggagtacctgcagattaccagcactcccatcgtggtggattgcagcacctacgtgtgcaacggcaacgtgcggtgcgtggagctgctgaagcagtacaccagcgcttgcaagaccatcgaggacgccctgcggaacagcgccatgctggagtctgccgacgtgagcgagatgctgaccttcgacaagaaggccttcacactggccaacgtgagcagctttggcgactacaacctgagcagcgtgattccaagccttcctcggagcggctctagggtggccggccggtctgcaatcgaggacatcttgttcagcaagctggtgaccagcggcctgggcacagtggacgccgactacaagaagtgcaccaaaggcctgagcatcgccgatctggcctgcgcccagtactacaacggaatcatggtgcttccaggggctgccgacgcagagcggatggccatgtacaccggcagcctgatcggcggaatcgccctgggcggactgaccagcgctgccagcatcccctttagcctggccatccagagccggctgaactacgtggcccttcagactgacgtgctgcaggagaaccagcggatcctggccgccagcttcaacaaggccatgaccaacatcgtggacgccttcacaggcgtgaacgacgccatcacccagaccagccaggccctgcagaccgtggccaccgccctcaacaagatccaggacgtggtgaaccagcagggcaacagcctgaaccacctgacctctcagctgcggcagaacttccaggccatcagcagtagcatccaagcaatctacgaccggctggatccaccacaggccgaccagcaggtggaccggctgataaccggccgcctggctgcccttaacgtgttcgtgagccataccctgaccaagtacaccgaggtgcgggcctctcggcagctggcccagcagaaggtgaacgagtgcgtgaagagccagagcaagcggtacggcttctgcggcaacgggacccacatcttcagccttgtgaacgccgcccctgaaggcctggtgttcctgcacaccgtgctactgcccacccagtacaaggacgttgaggcctggagcggcctgtgcgtggacgggatcaacggctacgtgctccggcagcccaacctggccctgtacaaggagggcaactactaccggatcaccagccggatcatgttcgagccccggatccccaccatcgccgacttcgtgcagatcgagaactgcaacgtgacctttgtgaacattagccggagcgagctgcagaccatcgtgcccgagtacatcgacgtgaacaagaccctgcaggagctgagctacaagctgcccaactacaccgttcccgacctggtggtggagcagtacaaccagaccatcctgaacctgacaagcgagataagcaccctggagaacaagagcgccgagctgaattacaccgtgcagaagctccagacactgatcgacaacatcaacagcactctggtggacctgaagtggctgaaccgggtggagacctacatcaagtggccctggtgggtgtggctgtgcatcagcgtggtgctgatcttcgtggtgagcatgctgctgctgtgctgctgcagcaccggctgctgcggcttcttcagctgcttcgccagcagcatccggggttgctgcgagagcaccaagttaccctactacgacgtggagaagatccacatccag

SEQ ID NO: 11 - protein, 1171 aa, synthetic construct
MFVLLVAYALLHIAGCQTINGTNTSHSVCNGCVGHSENVFAVESGGYIPSNFAFNNWFLLTNTSSVVDGVVRSFQPLLLNCLWSVSGSQFITGFVYFNGTGKGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGNVEAVNFNVTNAATTVCTVALASYADVLVNVSQTAIANIIYCNSVINRLRCDQLSFDVPDGFYSTSPIQPVELPVSIVSLPVYHKHTFIVLYVNFEHQRGPGKCYNCRPAVINITLANFNETKGPLCVDTSHFTTQFVDNVKLARWSASIDTGNCPFSFGKVINFVKFGSVCFSLKYIPGGCAMPIMANLVNYKSHNIGSLYVSWSDGDVITGVPKPVEGVSSFMNVTLNKCTKYNIYDVSGVGVIRISNDTFLNGITYTSTSGNLLGFKDVTNGTIYSITPCNPPDQLVVYQQAVVGAMLSENFTSYGFSNVVEMPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSAMLESADVSEMLTFDKKAFTLANVSSFGDYNLSSVIPSLPRSGSRVAGRSAIEDILFSKLVTSGLGTVDADYKKCTKGLSIADLACAQYYNGIMVLPGAADAERMAMYTGSLIGGIALGGLTSAASIPFSLAIQSRLNYVALQTDVLQENQRILAASFNKAMTNIVDAFTGVNDAITQTSQALQTVATALNKIQDVVNQQGNSLNHLTSQLRQNFQAISSSIQAIYDRLDPPQADQQVDRLITGRLAALNVFVSHTLTKYTEVRASRQLAQQKVNECVKSQSKRYGFCGNGTHIFSLVNAAPEGLVFLHTVLLPTQYKDVEAWSGLCVDGINGYVLRQPNLALYKEGNYYRITSRIMFEPRIPTIADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 12 - RNA, 4241 nt, synthetic construct
  source 1..4241: mol_type = other RNA
gggaaataagagagaaaagaagagtaagaagaaatataagaccccggcgccgccaccatgaagctgttcctgatcttactgatcctgcccctggtgagctgcttctctacctgcaacagcaacgccagcatcagcatgctccaactgggcgtgcctgacaacagctctaccatcgtgaccggcctgctgcccgtgcactggatctgcgccaaccaaagcaccagcacctaccccgcaaacggcttcttctacatcgacgtgggcaagcaccggagcgccttcgccctgcacagcggctactacgacgccaaccagtactatatctacctgaccaacaacatcagcctgaacgccccagtgaccctgaagatctgcaagttcggcaacaccagcttcgacttcctgagcaacgtgagtaccagccaccactgcattgtgaactcaagcttcaccgagcagctgggagtgcccctgggcatcaccatcagcggcgagaccgtgcggctgcacctgtacaacgccacccggaccttctacgtgcccgccgcctacaagctgaccaagctgagcgtgaagtgctacttcagcgagagctgcgtgtttagcgtggtaaacgccaccgtgaccgtgaacgtgaccaccctgaacggccggatcgtgaactacaccgtgtgcgacgactgcaacggctacaccgacaacatctttagcgtgcagcaggacgggcggatccccaacggctttagcttcaacaactggttcctgctgaccaacggcagcaccctggtggacggcgtgagccggctgtaccagcccctgcggctgacctgcctgtggcccgtgcccggtctgaagagctccaccggcttcgtgtacttcaacgccactggcagcgacgtgaattgtaacggctatcagcaccacagcgtggccgacgtgatgcggtacaacctgaacttcagctcaaacagcgtggacaaccttaagagcggcgtgatcgtgtttaagacactgcagtacgacgtgctcttctactgcagcaacagcagcagcggcgtgctggacaccaccatacccttcggccccagcagccagccctactattgcttcatcaacagcaccatcaacaccacccacgtgtctaccttcgtgggcgtgctgccgcccatcgtgcgggagatcgttgtggcccggaccggccagttctacatcaacggcttcaagtacttcgaccttggcttcatcgaggccgtgaacttcaacgtgacaaccgccagcgccaccgacttctggaccgtggccttcgccacctttgtggacgtgctggtcaacgtgagcgccaccaacatccagaatctgctgtactgcgacagccccttcgagaagctgcagtgcgagcacctgcagttcggcctgcaggacggcttctacagcgccaacttcctggacgacaacgtgctgcccgacacctacgtcgccctgcccatctactaccagcacaccgacatcaacttcaccgccaccgccagcttcggaggcagctgttacgtgtgcaagccccaccaggtgaacatcagtctgaacggcaacactagtgtgtgcgtgcggaccagccacttcagcatacggtacatctacaaccgggtgaagagcggaagccccggcgacagcagctggcacatctacctaaagagcggcacctgccccttcagctttagcaagctgaacaacttccagaagttcaagaccatttgcttcagcaccgtggcagtgcccggcagctgtaactttcccctggaagccacctggcactacaccagctacaccatcgtgggcgccctgtacgtgacctggagcgagggcaacagcatcaccggcgtgccctaccccgtgagcggcatccgggagttcagcaacctggtgctgaataactgcacgaagtacaacatctacgattacgtgggcaccggcatcatccggtctagcaaccagagcctggccggcggcatcacctacgtaagcaacagcggcaacctgctgggcttcaagaacgttagcaccggcaacatcttcatcgtgacgccctgcaaccagcccgaccaggtggccgtgtaccagcagagcatcatcggcgccatgaccgccgtgaacgagagccggtacggtctgcagaacctgctgcagctgcccaacttctactacgtgagcaacggcggcaacaactgcaccaccgctgtgatgacctacagcaacttcggcatctgcgccgacggcagcctgatccccgtgcggccccggaacagcagcgataacggcatcagcgccatcatcaccgccaatctgagcatccccagcaactggaccaccagcgtgcaggtggagtacctgcagatcacctcaactcccatcgtggtggactgcgccacctacgtgtgtaacggcaatccccggtgcaagaacctgttgaagcagtacaccagcgcctgcaagaccatcgaggacgccctgaggctgagcgcccacctggagaccaacgacgtgagcagcatgcttaccttcgactccaacgccttcagcctggccaacgtgactagcttcggcgactataacctgagctctgtgctgccccagcggaacatccggagctcccggatcgccggcagaagcgccctggaggacctgctgttcagcaaggtggtgaccagcggcctgggcaccgtggacgtggattacaagagctgcaccaagggcctgagcatcgccgatctggcctgcgcccagtactacaacggcattatggtgttacccggcgttgccgacgccgagcggatggccatgtacaccggcagtctgatcggcggcatggtgctgggcggcctgacaagcgccgccgccatcccctttagcctggctctgcaggcccggctgaactacgtggccctgcaaaccgacgtgctgcaggagaaccagaagatcctggccgcctcattcaacaaggccatcaacaacattgtggccagcttcagcagcgtgaacgacgccatcacccagaccgccgaggccatccacaccgtcaccatcgccctgaacaagatccaggacgtggtgaaccagcagggctccgcccttaaccacctgaccagccagctgcggcacaactttcaggccatcagcaatagcatccaggccatctacgaccggctggacccaccccaggccgaccagcaggtggaccggctgatcaccggccggttggccgcactgaacgccttcgtgagccaggtgctgaacaagtacaccgaggtgcggagctctcggcggctggcccagcagaagatcaacgagtgcgtgaagagccagagcaaccggtacggcttctgcggcaacggcacccacatcttcagcatcgtgaacagcgcccctgacggcctactgttcctgcacaccgtgctgctgcccaccgactacaagaacgtgaaggcctggagcggcatttgcgtggacggcatctacggctacgtgctgcggcagcccaacttggtgctgtacagcgacaacggcgtgttccggatcaccagccgggtgatgttccagccccgactgcccgttctgagcgactttgtgcagatctacaactgcaacgtgaccttcgtgaacattagccgggttaagctgcataccgtgatccccgactacgtggacgttaacaagaccctgcaggagttcgcccagaacttgcccaagtacgtgaagcccaatttcgacctgacacccttcaacctgacctacctgaacctgagcagcgagctgaagcagctggaggccaagaccgccagcctgttccagaccaccgtggagctgcagggcctgatcgaccagatcaatagcacatacgtggacctgaagctgctgaaccggttcgagaactacatcaagtggccctggtgggtgtggctgatcatcagcgtggtgttcgtggtgctgctgagcctgctggtgttctgctgcctgagcaccggctgctgtggctgctgcaactgcctgacctccagcatgcggggctgctgcgactgcggctccaccaagctgccctactacgagttcgagaaggtgcacgtgcagtgataataggctggagcctcggtggcctagcttcttgccccttgggcctccccccagcccctcctccccttcctgcacccgtacccccgtggtctttgaataaagtctgagtgggcggc

SEQ ID NO: 13 - RNA, 4065 nt, synthetic construct
  source 1..4065: mol_type = other RNA
atgaagctgttcctgatcttactgatcctgcccctggtgagctgcttctctacctgcaacagcaacgccagcatcagcatgctccaactgggcgtgcctgacaacagctctaccatcgtgaccggcctgctgcccgtgcactggatctgcgccaaccaaagcaccagcacctaccccgcaaacggcttcttctacatcgacgtgggcaagcaccggagcgccttcgccctgcacagcggctactacgacgccaaccagtactatatctacctgaccaacaacatcagcctgaacgccccagtgaccctgaagatctgcaagttcggcaacaccagcttcgacttcctgagcaacgtgagtaccagccaccactgcattgtgaactcaagcttcaccgagcagctgggagtgcccctgggcatcaccatcagcggcgagaccgtgcggctgcacctgtacaacgccacccggaccttctacgtgcccgccgcctacaagctgaccaagctgagcgtgaagtgctacttcagcgagagctgcgtgtttagcgtggtaaacgccaccgtgaccgtgaacgtgaccaccctgaacggccggatcgtgaactacaccgtgtgcgacgactgcaacggctacaccgacaacatctttagcgtgcagcaggacgggcggatccccaacggctttagcttcaacaactggttcctgctgaccaacggcagcaccctggtggacggcgtgagccggctgtaccagcccctgcggctgacctgcctgtggcccgtgcccggtctgaagagctccaccggcttcgtgtacttcaacgccactggcagcgacgtgaattgtaacggctatcagcaccacagcgtggccgacgtgatgcggtacaacctgaacttcagctcaaacagcgtggacaaccttaagagcggcgtgatcgtgtttaagacactgcagtacgacgtgctcttctactgcagcaacagcagcagcggcgtgctggacaccaccatacccttcggccccagcagccagccctactattgcttcatcaacagcaccatcaacaccacccacgtgtctaccttcgtgggcgtgctgccgcccatcgtgcgggagatcgttgtggcccggaccggccagttctacatcaacggcttcaagtacttcgaccttggcttcatcgaggccgtgaacttcaacgtgacaaccgccagcgccaccgacttctggaccgtggccttcgccacctttgtggacgtgctggtcaacgtgagcgccaccaacatccagaatctgctgtactgcgacagccccttcgagaagctgcagtgcgagcacctgcagttcggcctgcaggacggcttctacagcgccaacttcctggacgacaacgtgctgcccgacacctacgtcgccctgcccatctactaccagcacaccgacatcaacttcaccgccaccgccagcttcggaggcagctgttacgtgtgcaagccccaccaggtgaacatcagtctgaacggcaacactagtgtgtgcgtgcggaccagccacttcagcatacggtacatctacaaccgggtgaagagcggaagccccggcgacagcagctggcacatctacctaaagagcggcacctgccccttcagctttagcaagctgaacaacttccagaagttcaagaccatttgcttcagcaccgtggcagtgcccggcagctgtaactttcccctggaagccacctggcactacaccagctacaccatcgtgggcgccctgtacgtgacctggagcgagggcaacagcatcaccggcgtgccctaccccgtgagcggcatccgggagttcagcaacctggtgctgaataactgcacgaagtacaacatctacgattacgtgggcaccggcatcatccggtctagcaaccagagcctggccggcggcatcacctacgtaagcaacagcggcaacctgctgggcttcaagaacgttagcaccggcaacatcttcatcgtgacgccctgcaaccagcccgaccaggtggccgtgtaccagcagagcatcatcggcgccatgaccgccgtgaacgagagccggtacggtctgcagaacctgctgcagctgcccaacttctactacgtgagcaacggcggcaacaactgcaccaccgctgtgatgacctacagcaacttcggcatctgcgccgacggcagcctgatccccgtgcggccccggaacagcagcgataacggcatcagcgccatcatcaccgccaatctgagcatccccagcaactggaccaccagcgtgcaggtggagtacctgcagatcacctcaactcccatcgtggtggactgcgccacctacgtgtgtaacggcaatccccggtgcaagaacctgttgaagcagtacaccagcgcctgcaagaccatcgaggacgccctgaggctgagcgcccacctggagaccaacgacgtgagcagcatgcttaccttcgactccaacgccttcagcctggccaacgtgactagcttcggcgactataacctgagctctgtgctgccccagcggaacatccggagctcccggatcgccggcagaagcgccctggaggacctgctgttcagcaaggtggtgaccagcggcctgggcaccgtggacgtggattacaagagctgcaccaagggcctgagcatcgccgatctggcctgcgcccagtactacaacggcattatggtgttacccggcgttgccgacgccgagcggatggccatgtacaccggcagtctgatcggcggcatggtgctgggcggcctgacaagcgccgccgccatcccctttagcctggctctgcaggcccggctgaactacgtggccctgcaaaccgacgtgctgcaggagaaccagaagatcctggccgcctcattcaacaaggccatcaacaacattgtggccagcttcagcagcgtgaacgacgccatcacccagaccgccgaggccatccacaccgtcaccatcgccctgaacaagatccaggacgtggtgaaccagcagggctccgcccttaaccacctgaccagccagctgcggcacaactttcaggccatcagcaatagcatccaggccatctacgaccggctggacccaccccaggccgaccagcaggtggaccggctgatcaccggccggttggccgcactgaacgccttcgtgagccaggtgctgaacaagtacaccgaggtgcggagctctcggcggctggcccagcagaagatcaacgagtgcgtgaagagccagagcaaccggtacggcttctgcggcaacggcacccacatcttcagcatcgtgaacagcgcccctgacggcctactgttcctgcacaccgtgctgctgcccaccgactacaagaacgtgaaggcctggagcggcatttgcgtggacggcatctacggctacgtgctgcggcagcccaacttggtgctgtacagcgacaacggcgtgttccggatcaccagccgggtgatgttccagccccgactgcccgttctgagcgactttgtgcagatctacaactgcaacgtgaccttcgtgaacattagccgggttaagctgcataccgtgatccccgactacgtggacgttaacaagaccctgcaggagttcgcccagaacttgcccaagtacgtgaagcccaatttcgacctgacacccttcaacctgacctacctgaacctgagcagcgagctgaagcagctggaggccaagaccgccagcctgttccagaccaccgtggagctgcagggcctgatcgaccagatcaatagcacatacgtggacctgaagctgctgaaccggttcgagaactacatcaagtggccctggtgggtgtggctgatcatcagcgtggtgttcgtggtgctgctgagcctgctggtgttctgctgcctgagcaccggctgctgtggctgctgcaactgcctgacctccagcatgcggggctgctgcgactgcggctccaccaagctgccctactacgagttcgagaaggtgcacgtgcag

SEQ ID NO: 14 - protein, 1355 aa, synthetic construct
MKLFLILLILPLVSCFSTCNSNASISMLQLGVPDNSSTIVTGLLPVHWICANQSTSTYPANGFFYIDVGKHRSAFALHSGYYDANQYYIYLTNNISLNAPVTLKICKFGNTSFDFLSNVSTSHHCIVNSSFTEQLGVPLGITISGETVRLHLYNATRTFYVPAAYKLTKLSVKCYFSESCVFSVVNATVTVNVTTLNGRIVNYTVCDDCNGYTDNIFSVQQDGRIPNGFSFNNWFLLTNGSTLVDGVSRLYQPLRLTCLWPVPGLKSSTGFVYFNATGSDVNCNGYQHHSVADVMRYNLNFSSNSVDNLKSGVIVFKTLQYDVLFYCSNSSSGVLDTTIPFGPSSQPYYCFINSTINTTHVSTFVGVLPPIVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPDTYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDPPQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRITSRVMFQPRLPVLSDFVQIYNCNVTFVNISRVKLHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 15 - RNA, 4235 nt, synthetic construct
  source 1..4235: mol_type = other RNA
gggaaataagagagaaaagaagagtaagaagaaatataagaccccggcgccgccaccatgttccttatcctgctgatcagcctgcccaccgccttcgccgtgatcggcgacctgaagtgcaccagcgacaacatcaatgacaaggataccggaccgcctcccatcagcaccgacaccgtggacgtgaccaacggcctgggcacctactacgtgctggaccgggtgtacctgaataccaccctgttcctgaacggctattaccccaccagcggcagcacctaccggaatatggcgctgaagggcagcgtgctgctgagcagactgtggttcaaaccacccttcctgagcgactttatcaacggcattttcgcgaaggtgaagaacaccaaggtgatcaaggacagagtgatgtacagcgagttccccgccatcaccattgggagcaccttcgtcaacaccagctactctgtggtggtgcagccccggaccatcaacagcacccaggacggtgacaacaagctgcagggcctgctggaggtgagcgtgtgccagtacaacatgtgcgagtatccccagaccatctgccaccccaacctgggcaaccacagaaaggagctgtggcacctggacaccggagtggtgagctgcctgtacaagcggaacttcacctacgacgtgaacgctgactacctgtacttccacttctaccaggagggcggcaccttctacgcctacttcaccgataccggcgtggtgaccaagttcctgttcaacgtgtacctgggcatggccctgagccactactacgttatgcccctgacctgtaacagcaagctgaccctggagtactgggtgacccctctgaccagccggcagtacctgctggccttcaaccaggacggcatcatcttcaacgccgtggactgcatgtcagacttcatgagcgagatcaagtgcaagacccagagcatcgcccctcccaccggcgtgtacgagctgaacggttacaccgtgcagcccatcgccgacgtgtaccggcggaagcccaacctgcccaactgcaacatcgaggcctggctgaacgacaagagcgtgcccagcccactgaactgggagcggaagaccttcagtaactgcaacttcaacatgagcagcctgatgagcttcatccaggccgactcattcacctgcaacaacatcgacgccgccaagatctacggcatgtgcttcagcagcatcaccatcgacaagttcgccatcccaaacggccggaaggtggacttacagctgggcaatctgggctacctgcagagcttcaactaccggatcgacacgaccgccacctcctgccagttatactacaatctgcctgccgccaacgtgagcgtgagccgcttcaaccccagcacctggaacaagcggttcggcttcatcgaggacagcgtgttcaaacccagacccgccggcgtgctgacaaaccacgacgtggtgtacgcccagcactgctttaaggcccctaagaacttctgcccctgcaagctgaatggcagctgcgtcgggtctggccccggaaagaacaacgggattggcacctgccccgctggcaccaactacctgacctgcgacaacctctgcacccctgaccccatcaccttcaccggcacctacaagtgtcctcagaccaagagcctggtgggcatcggcgagcactgcagcggcctggcagtgaagagcgactactgcggcggcaacagctgcacctgcagaccccaggccttcctgggctggagcgccgacagctgcctgcagggcgacaagtgcaacatcttcgccaatttcatcctgcacgacgtcaacagcggactgacctgcagcaccgacctgcagaaggccaacaccgacatcatcctgggcgtgtgcgtgaactacgacctgtacggcatcctgggccagggcatcttcgtggaggtcaacgccacctattacaacagctggcagaacctgctgtacgacagcaacggtaatttatacggcttccgggactacatcaccaaccggaccttcatgatccggagctgctacagcggccgggtgagcgcagctttccacgccaacagcagcgagcccgccctgctgtttcggaacatcaagtgtaactacgtgttcaacaacagcctgacccggcagctgcagcctatcaactacttcgacagctacctgggctgcgtggtgaacgcatacaacagcactgccatcagcgtgcagacctgcgacctgaccgtgggctccgggtactgcgtggactacagcaagaacggcggctccggcggagctatcaccaccggctaccggttcaccaacttcgagcccttcaccgtgaacagcgtgaacgacagcctggagcccgtgggcggactgtacgagatccagatccccagcgagttcaccatcggcaacatggtggagttcatccagacctcaagccccaaggtgaccatcgactgcgccgccttcgtgtgcggcgactacgccgcctgcaagagccagctggtggagtacggcagcttctgcgacaatatcaacgccatcctgaccgaggtgaacgaactgctggacaccacccagttacaggtggccaacagtctgatgaacggcgtgaccctgagtaccaagctgaaggacggagtgaacttcaacgtggacgacatcaatttcagccccgtgctgggctgcctgggcagcgagtgcagcaaggccagtagccggagcgccatagaggacctgctgttcgacaaggtgaaactgagcgacgtgggcttcgtggaggcctacaacaactgcaccggtggcgccgagatccgggacctgatctgcgtgcagagctacaagggcatcaaagtgctgccacctctgctgagcgagaaccagatcagcggctacaccctcgccgccaccagcgctagcctgtttcctccttggactgctgccgcaggcgtgcccttctacctgaacgtgcagtaccggattaacggcctcggtgtgaccatggacgtgctgagccagaaccagaagctgatcgccaacgccttcaacaacgccctgtacgccatccaggagggattcgacgccaccaacagcgccctggtgaagatccaggcggtagtgaacgccaacgctgaggccctgaacaacctgctgcaacagctgagcaaccggttcggcgccatcagcgccagcctgcaggagatcctgagccggctggatccaccagaggccgaggcccagatcgaccggctgatcaacggccggctgaccgccctgaacgcctacgtgagccagcagctgagcgacagcaccctggtcaagttcagcgccgcccaggccatggagaaagtgaacgagtgcgtgaagagccagagcagccggatcaacttctgcggcaacggcaaccacatcatcagcctggtgcagaacgccccttacggcctgtactttatccacttcagctacgtgcccaccaagtacgtgaccgccagagtgagccccggcctgtgcatcgccggcgaccggggaatcgctcccaagagcggctacttcgtgaacgtgaacaacacctggatgtacaccggcagcggatactactaccccgagcccatcaccgagaacaacgtggtggtgatgagcacctgcgcagtgaactacaccaaggctccttacgtgatgctgaacacctctatcccaaatctgcccgacttcaaggaggagctggaccagtggttcaagaaccagaccagcgtggcccctgatctgagcctggactacattaacgtgaccttcctggacctgcaggtggagatgaaccggctgcaggaggccatcaaggtgctgaaccagtcatacatcaacctgaaggacatcggcacctacgagtactacgtgaagtggccctggtacgtgtggctgctgatctgcctggccggcgtggccatgctggtgctgctgttcttcatctgctgctgcaccggctgcggcaccagctgcttcaagaagtgcggcggctgctgcgacgactacaccggataccaggagctggtgatcaagaccagccacgacgactgataataggctggagcctcggtggcctagcttcttgccccttgggcctccccccagcccctcctccccttcctgcacccgtacccccgtggtctttgaataaagtctgagtgggcggc

SEQ ID NO: 16 - RNA, 4059 nt, synthetic construct
  source 1..4059: mol_type = other RNA
atgttccttatcctgctgatcagcctgcccaccgccttcgccgtgatcggcgacctgaagtgcaccagcgacaacatcaatgacaaggataccggaccgcctcccatcagcaccgacaccgtggacgtgaccaacggcctgggcacctactacgtgctggaccgggtgtacctgaataccaccctgttcctgaacggctattaccccaccagcggcagcacctaccggaatatggcgctgaagggcagcgtgctgctgagcagactgtggttcaaaccacccttcctgagcgactttatcaacggcattttcgcgaaggtgaagaacaccaaggtgatcaaggacagagtgatgtacagcgagttccccgccatcaccattgggagcaccttcgtcaacaccagctactctgtggtggtgcagccccggaccatcaacagcacccaggacggtgacaacaagctgcagggcctgctggaggtgagcgtgtgccagtacaacatgtgcgagtatccccagaccatctgccaccccaacctgggcaaccacagaaaggagctgtggcacctggacaccggagtggtgagctgcctgtacaagcggaacttcacctacgacgtgaacgctgactacctgtacttccacttctaccaggagggcggcaccttctacgcctacttcaccgataccggcgtggtgaccaagttcctgttcaacgtgtacctgggcatggccctgagccactactacgttatgcccctgacctgtaacagcaagctgaccctggagtactgggtgacccctctgaccagccggcagtacctgctggccttcaaccaggacggcatcatcttcaacgccgtggactgcatgtcagacttcatgagcgagatcaagtgcaagacccagagcatcgcccctcccaccggcgtgtacgagctgaacggttacaccgtgcagcccatcgccgacgtgtaccggcggaagcccaacctgcccaactgcaacatcgaggcctggctgaacgacaagagcgtgcccagcccactgaactgggagcggaagaccttcagtaactgcaacttcaacatgagcagcctgatgagcttcatccaggccgactcattcacctgcaacaacatcgacgccgccaagatctacggcatgtgcttcagcagcatcaccatcgacaagttcgccatcccaaacggccggaaggtggacttacagctgggcaatctgggctacctgcagagcttcaactaccggatcgacacgaccgccacctcctgccagttatactacaatctgcctgccgccaacgtgagcgtgagccgcttcaaccccagcacctggaacaagcggttcggcttcatcgaggacagcgtgttcaaacccagacccgccggcgtgctgacaaaccacgacgtggtgtacgcccagcactgctttaaggcccctaagaacttctgcccctgcaagctgaatggcagctgcgtcgggtctggccccggaaagaacaacgggattggcacctgccccgctggcaccaactacctgacctgcgacaacctctgcacccctgaccccatcaccttcaccggcacctacaagtgtcctcagaccaagagcctggtgggcatcggcgagcactgcagcggcctggcagtgaagagcgactactgcggcggcaacagctgcacctgcagaccccaggccttcctgggctggagcgccgacagctgcctgcagggcgacaagtgcaacatcttcgccaatttcatcctgcacgacgtcaacagcggactgacctgcagcaccgacctgcagaaggccaacaccgacatcatcctgggcgtgtgcgtgaactacgacctgtacggcatcctgggccagggcatcttcgtggaggtcaacgccacctattacaacagctggcagaacctgctgtacgacagcaacggtaatttatacggcttccgggactacatcaccaaccggaccttcatgatccggagctgctacagcggccgggtgagcgcagctttccacgccaacagcagcgagcccgccctgctgtttcggaacatcaagtgtaactacgtgttcaacaacagcctgacccggcagctgcagcctatcaactacttcgacagctacctgggctgcgtggtgaacgcatacaacagcactgccatcagcgtgcagacctgcgacctgaccgtgggctccgggtactgcgtggactacagcaagaacggcggctccggcggagctatcaccaccggctaccggttcaccaacttcgagcccttcaccgtgaacagcgtgaacgacagcctggagcccgtgggcggactgtacgagatccagatccccagcgagttcaccatcggcaacatggtggagttcatccagacctcaagccccaaggtgaccatcgactgcgccgccttcgtgtgcggcgactacgccgcctgcaagagccagctggtggagtacggcagcttctgcgacaatatcaacgccatcctgaccgaggtgaacgaactgctggacaccacccagttacaggtggccaacagtctgatgaacggcgtgaccctgagtaccaagctgaaggacggagtgaacttcaacgtggacgacatcaatttcagccccgtgctgggctgcctgggcagcgagtgcagcaaggccagtagccggagcgccatagaggacctgctgttcgacaaggtgaaactgagcgacgtgggcttcgtggaggcctacaacaactgcaccggtggcgccgagatccgggacctgatctgcgtgcagagctacaagggcatcaaagtgctgccacctctgctgagcgagaaccagatcagcggctacaccctcgccgccaccagcgctagcctgtttcctccttggactgctgccgcaggcgtgcccttctacctgaacgtgcagtaccggattaacggcctcggtgtgaccatggacgtgctgagccagaaccagaagctgatcgccaacgccttcaacaacgccctgtacgccatccaggagggattcgacgccaccaacagcgccctggtgaagatccaggcggtagtgaacgccaacgctgaggccctgaacaacctgctgcaacagctgagcaaccggttcggcgccatcagcgccagcctgcaggagatcctgagccggctggatccaccagaggccgaggcccagatcgaccggctgatcaacggccggctgaccgccctgaacgcctacgtgagccagcagctgagcgacagcaccctggtcaagttcagcgccgcccaggccatggagaaagtgaacgagtgcgtgaagagccagagcagccggatcaacttctgcggcaacggcaaccacatcatcagcctggtgcagaacgccccttacggcctgtactttatccacttcagctacgtgcccaccaagtacgtgaccgccagagtgagccccggcctgtgcatcgccggcgaccggggaatcgctcccaagagcggctacttcgtgaacgtgaacaacacctggatgtacaccggcagcggatactactaccccgagcccatcaccgagaacaacgtggtggtgatgagcacctgcgcagtgaactacaccaaggctccttacgtgatgctgaacacctctatcccaaatctgcccgacttcaaggaggagctggaccagtggttcaagaaccagaccagcgtggcccctgatctgagcctggactacattaacgtgaccttcctggacctgcaggtggagatgaaccggctgcaggaggccatcaaggtgctgaaccagtcatacatcaacctgaaggacatcggcacctacgagtactacgtgaagtggccctggtacgtgtggctgctgatctgcctggccggcgtggccatgctggtgctgctgttcttcatctgctgctgcaccggctgcggcaccagctgcttcaagaagtgcggcggctgctgcgacgactacaccggataccaggagctggtgatcaagaccagccacgacgac

SEQ ID NO: 17 - protein, 1353 aa, synthetic construct
MFLILLISLPTAFAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNGGSGGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDPPEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 18 - RNA, 4235 nt, synthetic construct; identical to SEQ ID NO: 15
  source 1..4235: mol_type = other RNA

SEQ ID NO: 19 - RNA, 4059 nt, synthetic construct; identical to SEQ ID NO: 16
  source 1..4059: mol_type = other RNA

SEQ ID NO: 20 - protein, 1353 aa, synthetic construct; identical to SEQ ID NO: 17

SEQ ID NO: 21 - RNA, 3695 nt, synthetic construct
  source 1..3695: mol_type = other RNA
gggaaataagagagaaaagaagagtaagaagaaatataagaccccggcgccgccaccatgttcgtgctgctggtggcctacgccctgctgcacatcgccggctgccagaccaccaacggcctgaacaccagctactccgtttgcaacggctgcgtgggctacagcgagaacgtcttcgccgtggagagcggcggctacatccctagcgacttcgccttcaacaactggttcctgctgaccaacaccagctccgtggtggacggcgtggtgcggagcttccagcccctgctgctgaactgcctgtggagtgtgagcggcctgagattcaccaccggcttcgtgtacttcaacggcaccggcagaggcgactgcaagggcttcagcagcgacgtgctgtctgacgtgattcggtacaacctgaacttcgaggagaacctgcggcggggcaccatcctgttcaagaccagctacggcgtggtggtgttctactgcaccaacaacaccctggtgagcggcgacgcccacatccccttcggcaccgtgctgggcaacttctactgcttcgtgaacaccaccatcggcaacgagacgaccagcgccttcgtgggcgccctgcccaagaccgtgcgggagttcgtgatcagccggaccggccacttctacattaacggataccggtacttcaccctgggcaacgtggaggccgtgaactttaacgtgaccaccgccgagaccaccgacttctgcaccgtagccctggccagctacgccgacgtgctcgtaaacgtgagccagaccagcatcgccaacatcatctactgcaacagcgtgatcaatcggctgcggtgcgaccagctgagcttcgacgtgcccgacggcttctactctaccagccccatccagagcgtggaactgcccgtgagcatcgtgagcctgcccgtgtaccacaagcacaccttcatcgtgctgtacgtggacttcaagccccagagcggcggcggcaagtgcttcaactgctaccccgccggcgtgaacatcaccctggctaacttcaacgagaccaagggccctctgtgcgttgataccagccatttcaccaccaagtacgtggccgtgtacgccaacgtgggccggtggagcgccagcatcaacaccggcaactgccccttcagcttcggcaaggtgaacaacttcgtgaagttcggcagcgtgtgcttcagcctgaaggacatcccaggcggctgcgccatgcccatcgtggccaactgggcctacagcaagtactacaccatcggcagcttatacgtgagctggagcgacggcgacggcatcaccggcgtgccccagcccgtggagggcgtgtctagcttcatgaacgtgaccctggacaagtgcactaagtacaacatctacgacgtcagcggcgtgggcgtgatacgggtgagcaacgacaccttcctgaacggcatcacctacaccagcaccagcggcaacctgctgggcttcaaggacgtgacgaagggcaccatctacagcatcaccccttgcaaccctcctgaccagctggtggtgtaccagcaggccgtggtgggcgccatgttaagcgagaacttcacctcttacggcttcagcaacgtggtggagctgcccaagttcttctacgccagcaacggcacttacaactgcaccgacgccgtgctgacctactcaagcttcggcgtgtgcgccgacggcagcatcatcgccgtgcagccccggaacgtgagctacgacagcgtgagcgccatcgtgaccgccaacctgtctatccccagcaactggaccaccagcgtgcaggtggagtacctgcagattaccagcactcccatcgtggtggattgcagcacctacgtgtgcaacggcaacgtgcggtgcgtggagctgctgaagcagtacaccagcgcttgcaagaccatcgaggacgccctgcggaacagcgccagactggagtctgccgacgtgagcgagatgctgaccttcgacaagaaggccttcacactggccaacgtgagcagctttggcgactacaacctgagcagcgtgattccaagccttcctaccagcggctctagggtggccggccggtctgcaatcgaggacatcttgttcagcaagctggtgaccagcggcctgggcacagtggacgccgactacaagaagtgcaccaaaggcctgagcatcgccgatctggcctgcgcccagtactacaacggaatcatggtgcttccaggggtggccgacgcagagcggatggccatgtacaccggcagcctgatcggcggaatcgccctgggcggactgaccagcgctgtgagcatcccctttagcctggccatccaggcccggctgaactacgtggcccttcagactgacgtgctgcaggagaaccagaagatcctggccgccagcttcaacaaggccatgaccaacatcgtggacgccttcacaggcgtgaacgacgccatcacccagaccagccaggccctgcagaccgtggccaccgccctcaacaagatccaggacgtggtgaaccagcagggcaacagcctgaaccacctgacctctcagctgcggcagaacttccaggccatcagcagtagcatccaagcaatctacgaccggctggatccaccacaggccgaccagcaggtggaccggctgataaccggccgcctggctgcccttaacgtgttcgtgagccataccctgaccaagtacaccgaggtgcgggcctctcggcagctggcccagcagaaggtgaacgagtgcgtgaagagccagagcaagcggtacggcttctgcggcaacgggacccacatcttcagcatcgtgaacgccgcccctgaaggcctggtgttcctgcacaccgtgctactgcccacccagtacaaggacgttgaggcctggagcggcctgtgcgtggacgggaccaacggctacgtgctccggcagcccaacctggccctgtacaaggagggcaactactaccggatcaccagccggatcatgttcgagccccggatccccaccatggccgacttcgtgcagatcgagaactgcaacgtgacctttgtgaacattagccggagcgagctgcagaccatcgtgcccgagtacatcgacgtgaacaagaccctgcaggagctgagctacaagctgcccaactacaccgttcccgacctggtggtggagcagtacaaccagaccatcctgaacctgacaagcgagataagcaccctggagaacaagagcgccgagctgaattacaccgtgcagaagctccagacactgatcgacaacatcaacagcactctggtggacctgaagtggctgaaccgggtggagacctacatcaagtggccctggtgggtgtggctgtgcatcagcgtggtgctgatcttcgtggtgagcatgctgctgctgtgctgctgcagcaccggctgctgcggcttcttcagctgcttcgccagcagcatccggggttgctgcgagagcaccaagttaccctactacgacgtggagaagatccacatccagtgataataggctggagcctcggtggcctagcttcttgccccttgggcctccccccagcccctcctccccttcctgcacccgtacccccgtggtctttgaataaagtctgagtgggcggc

SEQ ID NO: 22 - RNA, 3519 nt, synthetic construct
  source 1..3519: mol_type = other RNA
atgttcgtgctgctggtggcctacgccctgctgcacatcgccggctgccagaccaccaacggcctgaacaccagctactccgtttgcaacggctgcgtgggctacagcgagaacgtcttcgccgtggagagcggcggctacatccctagcgacttcgccttcaacaactggttcctgctgaccaacaccagctccgtggtggacggcgtggtgcggagcttccagcccctgctgctgaactgcctgtggagtgtgagcggcctgagattcaccaccggcttcgtgtacttcaacggcaccggcagaggcgactgcaagggcttcagcagcgacgtgctgtctgacgtgattcggtacaacctgaacttcgaggagaacctgcggcggggcaccatcctgttcaagaccagctacggcgtggtggtgttctactgcaccaacaacaccctggtgagcggcgacgcccacatccccttcggcaccgtgctgggcaacttctactgcttcgtgaacaccaccatcggcaacgagacgaccagcgccttcgtgggcgccctgcccaagaccgtgcgggagttcgtgatcagccggaccggccacttctacattaacggataccggtacttcaccctgggcaacgtggaggccgtgaactttaacgtgaccaccgccgagaccaccgacttctgcaccgtagccctggccagctacgccgacgtgctcgtaaacgtgagccagaccagcatcgccaacatcatctactgcaacagcgtgatcaatcggctgcggtgcgaccagctgagcttcgacgtgcccgacggcttctactctaccagccccatccagagcgtggaactgcccgtgagcatcgtgagcctgcccgtgtaccacaagcacaccttcatcgtgctgtacgtggacttcaagccccagagcggcggcggcaagtgcttcaactgctaccccgccggcgtgaacatcaccctggctaacttcaacgagaccaagggccctctgtgcgttgataccagccatttcaccaccaagtacgtggccgtgtacgccaacgtgggccggtggagcgccagcatcaacaccggcaactgccccttcagcttcggcaaggtgaacaacttcgtgaagttcggcagcgtgtgcttcagcctgaaggacatcccaggcggctgcgccatgcccatcgtggccaactgggcctacagcaagtactacaccatcggcagcttatacgtgagctggagcgacggcgacggcatcaccggcgtgccccagcccgtggagggcgtgtctagcttcatgaacgtgaccctggacaagtgcactaagtacaacatctacgacgtcagcggcgtgggcgtgatacgggtgagcaacgacaccttcctgaacggcatcacctacaccagcaccagcggcaacctgctgggcttcaaggacgtgacgaagggcaccatctacagcatcaccccttgcaaccctcctgaccagctggtggtgtaccagcaggccgtggtgggcgccatgttaagcgagaacttcacctcttacggcttcagcaacgtggtggagctgcccaagttcttctacgccagcaacggcacttacaactgcaccgacgccgtgctgacctactcaagcttcggcgtgtgcgccgacggcagcatcatcgccgtgcagccccggaacgtgagctacgacagcgtgagcgccatcgtgaccgccaacctgtctatccccagcaactggaccaccagcgtgcaggtggagtacctgcagattaccagcactcccatcgtggtggattgcagcacctacgtgtgcaacggcaacgtgcggtgcgtggagctgctgaagcagtacaccagcgcttgcaagaccatcgaggacgccctgcggaacagcgccagactggagtctgccgacgtgagcgagatgctgaccttcgacaagaaggccttcacactggccaacgtgagcagctttggcgactacaacctgagcagcgtgattccaagccttcctaccagcggctctagggtggccggccggtctgcaatcgaggacatcttgttcagcaagctggtgaccagcggcctgggcacagtggacgccgactacaagaagtgcaccaaaggcctgagcatcgccgatctggcctgcgcccagtactacaacggaatcatggtgcttccaggggtggccgacgcagagcggatggccatgtacaccggcagcctgatcggcggaatcgccctgggcggactgaccagcgctgtgagcatcccctttagcctggccatccaggcccggctgaactacgtggcccttcagactgacgtgctgcaggagaaccagaagatcctggccgccagcttcaacaaggccatgaccaacatcgtggacgccttcacaggcgtgaacgacgccatcacccagaccagccaggccctgcagaccgtggccaccgccctcaacaagatccaggacgtggtgaaccagcagggcaacagcctgaaccacctgacctctcagctgcggcagaacttccaggccatcagcagtagcatccaagcaatctacgaccggctggatccaccacaggccgaccagcaggtggaccggctgataaccggccgcctggctgcccttaacgtgttcgtgagccataccctgaccaagtacaccgaggtgcgggcctctcggcagctggcccagcagaaggtgaacgagtgcgtgaagagccagagcaagcggtacggcttctgcggcaacgggacccacatcttcagcatcgtgaacgccgcccctgaaggcctggtgttcctgcacaccgtgctactgcccacccagtacaaggacgttgaggcctggagcggcctgtgcgtggacgggaccaacggctacgtgctccggcagcccaacctggccctgtacaaggagggcaactactaccggatcaccagccggatcatgttcgagccccggatccccaccatggccgacttcgtgcagatcgagaactgcaacgtgacctttgtgaacattagccggagcgagctgcagaccatcgtgcccgagtacatcgacgtgaacaagaccctgcaggagctgagctacaagctgcccaactacaccgttcccgacctggtggtggagcagtacaaccagaccatcctgaacctgacaagcgagataagcaccctggagaacaagagcgccgagctgaattacaccgtgcagaagctccagacactgatcgacaacatcaacagcactctggtggacctgaagtggctgaaccgggtggagacctacatcaagtggccctggtgggtgtggctgtgcatcagcgtggtgctgatcttcgtggtgagcatgctgctgctgtgctgctgcagcaccggctgctgcggcttcttcagctgcttcgccagcagcatccggggttgctgcgagagcaccaagttaccctactacgacgtggagaagatccacatccag

SEQ ID NO: 23 - protein, 1173 aa, synthetic construct
MFVLLVAYALLHIAGCQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGNETTSAFVGALPKTVREFVISRTGHFYINGYRYFTLGNVEAVNFNVTTAETTDFCTVALASYADVLVNVSQTSIANIIYCNSVINRLRCDQLSFDVPDGFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSARLESADVSEMLTFDKKAFTLANVSSFGDYNLSSVIPSLPTSGSRVAGRSAIEDILFSKLVTSGLGTVDADYKKCTKGLSIADLACAQYYNGIMVLPGVADAERMAMYTGSLIGGIALGGLTSAVSIPFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDPPQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 24 - RNA, 4244 nt, synthetic construct
  source 1..4244: mol_type = other RNA
gggaaataagagagaaaagaagagtaagaagaaatataagaccccggcgccgccaccatgaagctgttcctgatcttactggtgctgcccctggccagctgcttcttcacctgcaacagcaacgccaacctgagcatgctccaactgggcgtgcctgacaacagctctaccatcgtgaccggcctgctgcccacccactggttctgcgccaaccaaagcaccagcgtgtacagcgcaaacggcttcttctacatcgacgtgggcaaccaccggagcgccttcgccctgcacaccggctactacgacgccaaccagtactatatctacgtgaccaacgagatcggcctgaacgccagcgtgaccctgaagatctgcaagttcagcaggaacaccaccttcgacttcctgagcaacgccagtagcagcttcgactgcattgtgaacctgctgttcaccgagcagctgggagcccctctgggcatcaccatcagcggcgagaccgtgcggctgcacctgtacaacgtgacccggaccttctacgtgcccgccgcctacaagctgaccaagctgagcgtgaagtgctacttcaactacagctgcgtgtttagcgtggtaaacgccaccgtgaccgtgaacgtgaccacccacaacggccgggtggtgaactacaccgtgtgcgacgactgcaacggctacaccgacaacatctttagcgtgcagcaggacgggcggatccccaacggctttcccttcaacaactggttcctgctgaccaacggcagcaccctggtggacggcgtgagccggctgtaccagcccctgcggctgacctgcctgtggcccgtgcccggtctgaagagctccaccggcttcgtgtacttcaacgccactggcagcgacgtgaattgtaacggctatcagcacaacagcgtggtggacgtgatgcggtacaacctgaacttcagcgccaacagcctggacaaccttaagagcggcgtgatcgtgtttaagacactgcagtacgacgtgctcttctactgcagcaacagcagcagcggcgtgctggacaccaccatacccttcggccccagcagccagccctactattgcttcatcaacagcaccatcaacaccacccacgtgtctaccttcgtgggcatcctgccgcccaccgtgcgggagatcgttgtggcccggaccggccagttctacatcaacggcttcaagtacttcgaccttggcttcatcgaggccgtgaacttcaacgtgacaaccgccagcgccaccgacttctggaccgtggccttcgccacctttgtggacgtgctggtcaacgtgagcgccaccaacatccagaatctgctgtactgcgacagccccttcgagaagctgcagtgcgagcacctgcagttcggcctgcaggacggcttctacagcgccaacttcctggacgacaacgtgctgcccgagacctacgtcgccctgcccatctactaccagcacaccgacatcaacttcaccgccaccgccagcttcggaggcagctgttacgtgtgcaagccccaccaggtgaacatcagtctgaacggcaacactagtgtgtgcgtgcggaccagccacttcagcatacggtacatctacaaccgggtgaagagcggaagccccggcgacagcagctggcacatctacctaaagagcggcacctgccccttcagctttagcaagctgaacaacttccagaagttcaagaccatttgcttcagcaccgtggaggtgcccggcagctgtaactttcccctggaagccacctggcactacaccagctacaccatcgtgggcgccctgtacgtgacctggagcgagggcaacagcatcaccggcgtgccctaccccgtgagcggcatccgggagttcagcaacctggtgctgaataactgcacgaagtacaacatctacgattacgtgggcaccggcatcatccggtctagcaaccagagcctggccggcggcatcacctacgtaagcaacagcggcaacctgctgggcttcaagaacgttagcaccggcaacatcttcatcgtgacgccctgcaaccagcccgaccaggtggccgtgtaccagcagagcatcatcggcgccatgaccgccgtgaacgagagccggtacggtctgcagaacctgctgcagctgcccaacttctactacgtgagcaacggcggcaacaactgcaccaccgctgtgatgacctacagcaacttcggcatctgcgccgacggcagcctgatccccgtgcggccccggaacagcagcgataacggcatcagcgccatcatcaccgccaatctgagcatccccagcaactggaccaccagcgtgcaggtggagtacctgcagatcacctcaactcccatcgtggtggactgcgccacctacgtgtgtaacggcaatccccggtgcaagaacctgttgaagcagtacaccagcgcctgcaagaccatcgaggacgccctgaggctgagcgcccacctggagaccaacgacgtgagcagcatgcttaccttcgactccaacgccttcagcctggccaacgtgactagcttcggcgactataacctgagctctgtgctgccccagcggaacatccggagctcccggatcgccggcagaagcgccctggaggacctgctgttcagcaaggtggtgaccagcggcctgggcaccgtggacgtggattacaagagctgcaccaagggcctgagcatcgccgatctggcctgcgcccagtactacaacggcattatggtgttacccggcgttgccgacgccgagcggatggccatgtacaccggcagtctgatcggcggcatggtgctgggcggcctgacaagcgccgccgccatcccctttagcctggctctgcaggcccggctgaactacgtggccctgcaaaccgacgtgctgcaggagaaccagaagatcctggccgcctcattcaacaaggccatcaacaacattgtggccagcttcagcagcgtgaacgacgccatcacccagaccgccgaggccatccacaccgtcaccatcgccctgaacaagatccaggacgtggtgaaccagcagggctccgcccttaaccacctgaccagccagctgcggcacaactttcaggccatcagcaatagcatccaggccatctacgaccggctggacccaccccaggccgaccagcaggtggaccggctgatcaccggccggttggccgcactgaacgccttcgtgagccaggtgctgaacaagtacaccgaggtgcggggctctcggcggctggcccagcagaagatcaacgagtgcgtgaagagccagagcaaccggtacggcttctgcggcaacggcacccacatcttcagcatcgtgaacagcgcccctgacggcctactgttcctgcacaccgtgctgctgcccaccgactacaagaacgtgaaggcctggagcggcatttgcgtggacggcatctacggctacgtgctgcggcagcccaacttggtgctgtacagcgacaacggcgtgttccgggtgaccagccgggtgatgttccagccccgactgcccgttctgagcgactttgtgcagatctacaactgcaacgtgaccttcgtgaacattagccgggttgagctgcataccgtgatccccgactacgtggacgttaacaagaccctgcaggagttcgcccagaacttgcccaagtacgtgaagcccaatttcgacctgacacccttcaacctgacctacctgaacctgagcagcgagctgaagcagctggaggccaagaccgccagcctgttccagaccaccgtggagctgcagggcctgatcgaccagatcaatagcacatacgtggacctgaagctgctgaaccggttcgagaactacatcaagtggccctggtgggtgtggctgatcatcagcgtggtgttcgtggtgctgctgagcctgctggtgttctgctgcctgagcaccggctgctgtggctgctgcaactgcctgacctccagcatgcggggctgctgcgactgcggctccaccaagctgccctactacgagttcgagaaggtgcacgtgcagtgataataggctggagcctcggtggcctagcttcttgccccttgggcctccccccagcccctcctccccttcctgcacccgtacccccgtggtctttgaataaagtctgagtgggcggc

SEQ ID NO: 25 - RNA, 4068 nt, synthetic construct
  source 1..4068: mol_type = other RNA
atgaagctgttcctgatcttactggtgctgcccctggccagctgcttcttcacctgcaacagcaacgccaacctgagcatgctccaactgggcgtgcctgacaacagctctaccatcgtgaccggcctgctgcccacccactggttctgcgccaaccaaagcaccagcgtgtacagcgcaaacggcttcttctacatcgacgtgggcaaccaccggagcgccttcgccctgcacaccggctactacgacgccaaccagtactatatctacgtgaccaacgagatcggcctgaacgccagcgtgaccctgaagatctgcaagttcagcaggaacaccaccttcgacttcctgagcaacgccagtagcagcttcgactgcattgtgaacctgctgttcaccgagcagctgggagcccctctgggcatcaccatcagcggcgagaccgtgcggctgcacctgtacaacgtgacccggaccttctacgtgcccgccgcctacaagctgaccaagctgagcgtgaagtgctacttcaactacagctgcgtgtttagcgtggtaaacgccaccgtgaccgtgaacgtgaccacccacaacggccgggtggtgaactacaccgtgtgcgacgactgcaacggctacaccgacaacatctttagcgtgcagcaggacgggcggatccccaacggctttcccttcaacaactggttcctgctgaccaacggcagcaccctggtggacggcgtgagccggctgtaccagcccctgcggctgacctgcctgtggcccgtgcccggtctgaagagctccaccggcttcgtgtacttcaacgccactggcagcgacgtgaattgtaacggctatcagcacaacagcgtggtggacgtgatgcggtacaacctgaacttcagcgccaacagcctggacaaccttaagagcggcgtgatcgtgtttaagacactgcagtacgacgtgctcttctactgcagcaacagcagcagcggcgtgctggacaccaccatacccttcggccccagcagccagccctactattgcttcatcaacagcaccatcaacaccacccacgtgtctaccttcgtgggcatcctgccgcccaccgtgcgggagatcgttgtggcccggaccggccagttctacatcaacggcttcaagtacttcgaccttggcttcatcgaggccgtgaacttcaacgtgacaaccgccagcgccaccgacttctggaccgtggccttcgccacctttgtggacgtgctggtcaacgtgagcgccaccaacatccagaatctgctgtactgcgacagccccttcgagaagctgcagtgcgagcacctgcagttcggcctgcaggacggcttctacagcgccaacttcctggacgacaacgtgctgcccgagacctacgtcgccctgcccatctactaccagcacaccgacatcaacttcaccgccaccgccagcttcggaggcagctgttacgtgtgcaagccccaccaggtgaacatcagtctgaacggcaacactagtgtgtgcgtgcggaccagccacttcagcatacggtacatctacaaccgggtgaagagcggaagccccggcgacagcagctggcacatctacctaaagagcggcacctgccccttcagctttagcaagctgaacaacttccagaagttcaagaccatttgcttcagcaccgtggaggtgcccggcagctgtaactttcccctggaagccacctggcactacaccagctacaccatcgtgggcgccctgtacgtgacctggagcgagggcaacagcatcaccggcgtgccctaccccgtgagcggcatccgggagttcagcaacctggtgctgaataactgcacgaagtacaacatctacgattacgtgggcaccggcatcatccggtctagcaaccagagcctggccggcggcatcacctacgtaagcaacagcggcaacctgctgggcttcaagaacgttagcaccggcaacatcttcatcgtgacgccctgcaaccagcccgaccaggtggccgtgtaccagcagagcatcatcggcgccatgaccgccgtgaacgagagccggtacggtctgcagaacctgctgcagctgcccaacttctactacgtgagcaacggcggcaacaactgcaccaccgctgtgatgacctacagcaacttcggcatctgcgccgacggcagcctgatccccgtgcggccccggaacagcagcgataacggcatcagcgccatcatcaccgccaatctgagcatccccagcaactggaccaccagcgtgcaggtggagtacctgcagatcacctcaactcccatcgtggtggactgcgccacctacgtgtgtaacggcaatccccggtgcaagaacctgttgaagcagtacaccagcgcctgcaagaccatcgaggacgccctgaggctgagcgcccacctggagaccaacgacgtgagcagcatgcttaccttcgactccaacgccttcagcctggccaacgtgactagcttcggcgactataacctgagctctgtgctgccccagcggaacatccggagctcccggatcgccggcagaagcgccctggaggacctgctgttcagcaaggtggtgaccagcggcctgggcaccgtggacgtggattacaagagctgcaccaagggcctgagcatcgccgatctggcctgcgcccagtactacaacggcattatggtgttacccggcgttgccgacgccgagcggatggccatgtacaccggcagtctgatcggcggcatggtgctgggcggcctgacaagcgccgccgccatcccctttagcctggctctgcaggcccggctgaactacgtggccctgcaaaccgacgtgctgcaggagaaccagaagatcctggccgcctcattcaacaaggccatcaacaacattgtggccagcttcagcagcgtgaacgacgccatcacccagaccgccgaggccatccacaccgtcaccatcgccctgaacaagatccaggacgtggtgaaccagcagggctccgcccttaaccacctgaccagccagctgcggcacaactttcaggccatcagcaatagcatccaggccatctacgaccggctggacccaccccaggccgaccagcaggtggaccggctgatcaccggccggttggccgcactgaacgccttcgtgagccaggtgctgaacaagtacaccgaggtgcggggctctcggcggctggcccagcagaagatcaacgagtgcgtgaagagccagagcaaccggtacggcttctgcggcaacggcacccacatcttcagcatcgtgaacagcgcccctgacggcctactgttcctgcacaccgtgctgctgcccaccgactacaagaacgtgaaggcctggagcggcatttgcgtggacggcatctacggctacgtgctgcggcagcccaacttggtgctgtacagcgacaacggcgtgttccgggtgaccagccgggtgatgttccagccccgactgcccgttctgagcgactttgtgcagatctacaactgcaacgtgaccttcgtgaacattagccgggttgagctgcataccgtgatccccgactacgtggacgttaacaagaccctgcaggagttcgcccagaacttgcccaagtacgtgaagcccaatttcgacctgacacccttcaacctgacctacctgaacctgagcagcgagctgaagcagctggaggccaagaccgccagcctgttccagaccaccgtggagctgcagggcctgatcgaccagatcaatagcacatacgtggacctgaagctgctgaaccggttcgagaactacatcaagtggccctggtgggtgtggctgatcatcagcgtggtgttcgtggtgctgctgagcctgctggtgttctgctgcctgagcaccggctgctgtggctgctgcaactgcctgacctccagcatgcggggctgctgcgactgcggctccaccaagctgccctactacgagttcgagaaggtgcacgtgcag

SEQ ID NO: 26 - protein, 1356 aa, synthetic construct
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDPPQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 27 - RNA, 4262 nt, synthetic construct
  source 1..4262: mol_type = other RNA
gggaaataagagagaaaagaagagtaagaagaaatataagaccccggcgccgccaccatgttcctgatcctgctgatcagcctgcccaccgccttcgccgtgatcggcgacctgaagtgccctctggagagccggaccggcagcctgaacaatatcgacaccggccctcccagcatcagcaccgccaccgtggacgtgaccaacggcctgggcacctactacgtgctggaccgggtgtacctgaacaccaccctgttcctgaacggctattaccccaccagcggcagcacctaccggaatatggcgctgaagggcaccgacaagctgagcaccctgtggttcaagccacccttcctgagcgactttatcaacggcatctttgccaaggtgaagaacaccaaggtgttcaaggacggcgtgatgtacagcgagttccccgccatcactatcggtagcacgttcgtgaacaccagctacagcgtggtggtacagccccggaccatcaacagcacccaggacggagtgaacaagctgcagggcctgctggaggtgagcgtgtgccagtacaacatgtgcgagtaccctcacaccatctgccaccccaacctcggcaaccacttcaaggagctgtggcacatggataccggcgtggtgagctgcctgtacaagcggaacttcacctacgacgtcaacgccacctacctgtacttccacttctaccaggaaggaggcaccttctacgcctacttcaccgacaccggagtggtgaccaagttcctgttcaacgtgtacctgggcatggccctgagccactactacgtcatgcccctgacttgtatcagccggcggaacatcggcttcaccctggagtactgggtgaccccactgaccagccggcagtacctgctggccttcaaccaggacggcatcatcttcaacgccgtggactgcatgagcgatttcatgagcgagatcaagtgcaagacccagagcatcgcgccacccaccggcgtgtacgagctgaacgggtacaccgtgcagcccatcgccgacgtgtaccggcggaagcccgacctgccaaactgcaacatcgaggcctggctgaacgacaagagcgtgccctcacccctgaactgggagcggaagaccttcagcaactgcaacttcaacatgagcagcctgatgagcttcatccaggctgacagcttcacctgcaacaacatcgacgccgccaagatctacggcatgtgcttcagctctatcaccatcgacaagttcgccatccccaacggccggaaggtggacctgcaactgggcaacctgggctacctgcagagcttcaactaccggatcgacacgaccgccaccagttgccagctctactacaatctgcccgccgccaacgtgagcgtgagccggttcaaccccagcacctggaacaagcggttcggcttcatcgaagacagcgtgttcaagccccagccagccggcgtgttcaccaaccacgacgtggtgtacgcccagcactgctttaaggcccctaagaacttctgcccctgcaagagtaacagcagcttgtgtgttggcagcggccccggcaagaacaacggcattggcacctgccccgctggcaccaactacctgacctgccacaacctgtgcaaccccgaccccatcaccttcaccggcccctacaagtgtccccagaccaagagcctggtgggcatcggcgagcactgcagcggcctggccgtgaagagcgactactgcggcggcaatctgtgcacctgccagccccaggccttcctgggctggagcgccgacagctgcctgcagggcgacaagtgcaacatcttcgccaacctgatactgcacgacgtgaatagcggcctgacctgtagcaccgacctgcagaaggccaacaccgacatcaagctcggcgtgtgcgttaactacgacctgtacggcatcagcggccagggcatcttcgtggaggtgaacgctacctactacaacagctggcagaacctgctgtacgacagcaacggcaatttgtacggcttccgggactacatcaccaaccggaccttcatgatccggagctgctacagcggacgggtgagcgccgctttccacgccaacagcagcgagcccgccctgctgtttcggaacatcaagtgtaactacgtgttcaacaacagcctgacccggcagttacagcccatcaactacttcgacagctacctgggctgcgtcgtgaacgcttacaacagcacagccatcagcgtgcagacctgcgacctgaccgtgggcagcgggtactgcgtggactacagcaagaaccggcggtctcggcgggctatcaccaccggctaccggttcaccaatttcgagcccttcaccgtgaacagcgtgaacgacagcctggagcccgtgggcggtctgtacgagatccagatccccagcgacttcaccatcggcaacatggaggagttcatccagaccagcagccccaaggtgaccatcgactgcgccgccttcgtgtgcggcgactacgccgcctgtaagagccagctggtggagtacggcagcttctgcgacaacatcaacgccatcctgaccgaggttaacgagctgctggacaccacccagctgcaggtggccaacagcctcatgaacggcgtgaccctgagcatcaagctgaaagacggggtgaacttcaacgtggacgacatcaacttcagcagcgtgctgggctgcctgggcagcgagtgcagcaaggcctccagccggagcgccatcgaggacctgctgttcgacaaggtgaaactgagcgacgtgggcttcgtggccgcctacaacaactgcactggcggcgccgagatccgggacctgatctgcgtgcaaagctacaagggcatcaaagtgcttccacccctgctgagcgagaaccagatcagcggctacaccctggctgccaccagcgccagcctgtttcctccctggacagcagctgccggcgtgcccttctacctgaacgtgcagtaccggattaacggactgggcgtgaccatggacgtgctgagccagaaccagaagctgatcgccaacgccttcaacaacgccctggacgccatccaggagggcttcgacgccaccaacagcgctctggtgaagatccaggccgtggtgaacgccaacgcagaggccctgaacaacctgctgcaacagctgagcaaccggttcggcgccatcagcagcagcctgcaggagatcctgagccggctggatcctcctgaggccgaggcccagatcgaccggctgatcaacggccgcctgaccgccctgaacgcctacgtgagccagcagctgagcgacagcaccctggtgaaattcagcgccgcccaggccatggagaaggtgaacgagtgcgtgaagtcacagagcagccggatcaacttctgcggcaacggaaaccacatcatcagcctggtgcagaacgcgccctacggtttgtacttcatccacttcagctacgtgcccaccaagtacgtgaccgcaaaggtgagccccggcctgtgtatcgccggcgaccggggcattgcacccaagagcggctacttcgtgaacgtgaacaacacctggatgtacaccggatctggctactactaccccgagcccatcaccgagaacaacgtggtggtgatgagcacctgcgccgtgaactacaccaaggccccttacgtgatgctgaataccagcatcccaaacctgcccgacttccgggaggagctggaccagtggtttaagaaccaaaccagcgtggcacccgacctgagcctggactacattaacgtgaccttcctggacttgcaggtggagatgaacaggctgcaggaggccatcaaggtgctgaaccagagctacatcaacctgaaggacatcggcacctacgagtactacgtgaagtggccctggtacgtgtggctgctgatctgcctggccggcgtggccatgctggtgctgctgttcttcatctgctgctgcaccggctgcggcaccagctgcttcaagaagtgcggcggctgctgcgacgactacaccggctatcaggagctggtgatcaagaccagccacgacgactgataataggctggagcctcggtggcctagcttcttgccccttgggcctccccccagcccctcctccccttcctgcacccgtacccccgtggtctttgaataaagtctgagtgggcggc

SEQ ID NO: 28 - RNA, 4086 nt, synthetic construct
  source 1..4086: mol_type = other RNA
atgttcctgatcctgctgatcagcctgcccaccgccttcgccgtgatcggcgacctgaagtgccctctggagagccggaccggcagcctgaacaatatcgacaccggccctcccagcatcagcaccgccaccgtggacgtgaccaacggcctgggcacctactacgtgctggaccgggtgtacctgaacaccaccctgttcctgaacggctattaccccaccagcggcagcacctaccggaatatggcgctgaagggcaccgacaagctgagcaccctgtggttcaagccacccttcctgagcgactttatcaacggcatctttgccaaggtgaagaacaccaaggtgttcaaggacggcgtgatgtacagcgagttccccgccatcactatcggtagcacgttcgtgaacaccagctacagcgtggtggtacagccccggaccatcaacagcacccaggacggagtgaacaagctgcagggcctgctggaggtgagcgtgtgccagtacaacatgtgcgagtaccctcacaccatctgccaccccaacctcggcaaccacttcaaggagctgtggcacatggataccggcgtggtgagctgcctgtacaagcggaacttcacctacgacgtcaacgccacctacctgtacttccacttctaccaggaaggaggcaccttctacgcctacttcaccgacaccggagtggtgaccaagttcctgttcaacgtgtacctgggcatggccctgagccactactacgtcatgcccctgacttgtatcagccggcggaacatcggcttcaccctggagtactgggtgaccccactgaccagccggcagtacctgctggccttcaaccaggacggcatcatcttcaacgccgtggactgcatgagcgatttcatgagcgagatcaagtgcaagacccagagcatcgcgccacccaccggcgtgtacgagctgaacgggtacaccgtgcagcccatcgccgacgtgtaccggcggaagcccgacctgccaaactgcaacatcgaggcctggctgaacgacaagagcgtgccctcacccctgaactgggagcggaagaccttcagcaactgcaacttcaacatgagcagcctgatgagcttcatccaggctgacagcttcacctgcaacaacatcgacgccgccaagatctacggcatgtgcttcagctctatcaccatcgacaagttcgccatccccaacggccggaaggtggacctgcaactgggcaacctgggctacctgcagagcttcaactaccggatcgacacgaccgccaccagttgccagctctactacaatctgcccgccgccaacgtgagcgtgagccggttcaaccccagcacctggaacaagcggttcggcttcatcgaagacagcgtgttcaagccccagccagccggcgtgttcaccaaccacgacgtggtgtacgcccagcactgctttaaggcccctaagaacttctgcccctgcaagagtaacagcagcttgtgtgttggcagcggccccggcaagaacaacggcattggcacctgccccgctggcaccaactacctgacctgccacaacctgtgcaaccccgaccccatcaccttcaccggcccctacaagtgtccccagaccaagagcctggtgggcatcggcgagcactgcagcggcctggccgtgaagagcgactactgcggcggcaatctgtgcacctgccagccccaggccttcctgggctggagcgccgacagctgcctgcagggcgacaagtgcaacatcttcgccaacctgatactgcacgacgtgaatagcggcctgacctgtagcaccgacctgcagaaggccaacaccgacatcaagctcggcgtgtgcgttaactacgacctgtacggcatcagcggccagggcatcttcgtggaggtgaacgctacctactacaacagctggcagaacctgctgtacgacagcaacggcaatttgtacggcttccgggactacatcaccaaccggaccttcatgatccggagctgctacagcggacgggtgagcgccgctttccacgccaacagcagcgagcccgccctgctgtttcggaacatcaagtgtaactacgtgttcaacaacagcctgacccggcagttacagcccatcaactacttcgacagctacctgggctgcgtcgtgaacgcttacaacagcacagccatcagcgtgcagacctgcgacctgaccgtgggcagcgggtactgcgtggactacagcaagaaccggcggtctcggcgggctatcaccaccggctaccggttcaccaatttcgagcccttcaccgtgaacagcgtgaacgacagcctggagcccgtgggcggtctgtacgagatccagatccccagcgacttcaccatcggcaacatggaggagttcatccagaccagcagccccaaggtgaccatcgactgcgccgccttcgtgtgcggcgactacgccgcctgtaagagccagctggtggagtacggcagcttctgcgacaacatcaacgccatcctgaccgaggttaacgagctgctggacaccacccagctgcaggtggccaacagcctcatgaacggcgtgaccctgagcatcaagctgaaagacggggtgaacttcaacgtggacgacatcaacttcagcagcgtgctgggctgcctgggcagcgagtgcagcaaggcctccagccggagcgccatcgaggacctgctgttcgacaaggtgaaactgagcgacgtgggcttcgtggccgcctacaacaactgcactggcggcgccgagatccgggacctgatctgcgtgcaaagctacaagggcatcaaagtgcttccacccctgctgagcgagaaccagatcagcggctacaccctggctgccaccagcgccagcctgtttcctccctggacagcagctgccggcgtgcccttctacctgaacgtgcagtaccggattaacggactgggcgtgaccatggacgtgctgagccagaaccagaagctgatcgccaacgccttcaacaacgccctggacgccatccaggagggcttcgacgccaccaacagcgctctggtgaagatccaggccgtggtgaacgccaacgcagaggccctgaacaacctgctgcaacagctgagcaaccggttcggcgccatcagcagcagcctgcaggagatcctgagccggctggatcctcctgaggccgaggcccagatcgaccggctgatcaacggccgcctgaccgccctgaacgcctacgtgagccagcagctgagcgacagcaccctggtgaaattcagcgccgcccaggccatggagaaggtgaacgagtgcgtgaagtcacagagcagccggatcaacttctgcggcaacggaaaccacatcatcagcctggtgcagaacgcgccctacggtttgtacttcatccacttcagctacgtgcccaccaagtacgtgaccgcaaaggtgagccccggcctgtgtatcgccggcgaccggggcattgcacccaagagcggctacttcgtgaacgtgaacaacacctggatgtacaccggatctggctactactaccccgagcccatcaccgagaacaacgtggtggtgatgagcacctgcgccgtgaactacaccaaggccccttacgtgatgctgaataccagcatcccaaacctgcccgacttccgggaggagctggaccagtggtttaagaaccaaaccagcgtggcacccgacctgagcctggactacattaacgtgaccttcctggacttgcaggtggagatgaacaggctgcaggaggccatcaaggtgctgaaccagagctacatcaacctgaaggacatcggcacctacgagtactacgtgaagtggccctggtacgtgtggctgctgatctgcctggccggcgtggccatgctggtgctgctgttcttcatctgctgctgcaccggctgcggcaccagctgcttcaagaagtgcggcggctgctgcgacgactacaccggctatcaggagctggtgatcaagaccagccacgacgac

SEQ ID NO: 29 - protein, 1362 aa, synthetic construct
MFLILLISLPTAFAVIGDLKCPLESRTGSLNNIDTGPPSISTATVDVTNGLGTYYVLDRVYLNTTLFLNGYYPTSGSTYRNMALKGTDKLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHMDTGVVSCLYKRNFTYDVNATYLYFHFYQEGGTFYAYFTDTGVVTKFLFNVYLGMALSHYYVMPLTCISRRNIGFTLEYWVTPLTSRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPQPAGVFTNHDVVYAQHCFKAPKNFCPCKSNSSLCVGSGPGKNNGIGTCPAGTNYLTCHNLCNPDPITFTGPYKCPQTKSLVGIGEHCSGLAVKSDYCGGNLCTCQPQAFLGWSADSCLQGDKCNIFANLILHDVNSGLTCSTDLQKANTDIKLGVCVNYDLYGISGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNRRSRRAITTGYRFTNFEPFTVNSVNDSLEPVGGLYEIQIPSDFTIGNMEEFIQTSSPKVTIDCAAFVCGDYAACKSQLVEYGSFCDNINAILTEVNELLDTTQLQVANSLMNGVTLSIKLKDGVNFNVDDINFSSVLGCLGSECSKASSRSAIEDLLFDKVKLSDVGFVAAYNNCTGGAEIRDLICVQSYKGIKVLPPLLSENQISGYTLAATSASLFPPWTAAAGVPFYLNVQYRINGLGVTMDVLSQNQKLIANAFNNALDAIQEGFDATNSALVKIQAVVNANAEALNNLLQQLSNRFGAISSSLQEILSRLDPPEAEAQIDRLINGRLTALNAYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMYTGSGYYYPEPITENNVVVMSTCAVNYTKAPYVMLNTSIPNLPDFREELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 30 - DNA, 29 bp, synthetic construct
  misc_feature 21..29: note = n is a, c, g, or t
  source 1..29: mol_type = other DNA
  misc_feature 21..29: note = May be repeated 1 or more times
taatacgactcactatagggnnnnnnnnn

SEQ ID NO: 31 - DNA, 18 bp, synthetic construct
  source 1..18: mol_type = other DNA
taatacgactcactatag

SEQ ID NO: 32 - DNA, 29 bp, synthetic construct
  misc_feature 20..29: note = n is a, c, g, or t
  source 1..29: mol_type = other DNA
  misc_feature 20..29: note = May be repeated 1 or more times
taatacgactcactataagnnnnnnnnnn

SEQ ID NO: 33 - DNA, 29 bp, synthetic construct
  misc_feature 20..29: note = n is a, c, g, or t
  source 1..29: mol_type = other DNA
  misc_feature 20..29: note = May be repeated 1or more times
attatgctgagtgatattcnnnnnnnnnn

SEQ ID NO: 34-35 - skipped (2 entries)

SEQ ID NO: 36 - RNA, 47 nt, synthetic construct
  source 1..47: mol_type = other RNA
gggaaataagagagaaaagaagagtaagaagaaatataagagccacc

SEQ ID NO: 37 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 38 - protein, 30 aa, synthetic construct
MDSKGSSQKGSRLLLLLVVSNLLLPQGVVG

SEQ ID NO: 39 - protein, 18 aa, synthetic construct
MDWTWILFLVAAATRVHS

SEQ ID NO: 40 - protein, 20 aa, synthetic construct
METPAQLLFLLLLWLPDTTG

SEQ ID NO: 41 - protein, 24 aa, synthetic construct
MLGSNSGQRVVFTILLLLVAPAYS

SEQ ID NO: 42 - protein, 17 aa, synthetic construct
MKCLLYLAFLFIGVNCA

SEQ ID NO: 43 - protein, 15 aa, synthetic construct
MWLVSLAIVTACAGA

SEQ ID NO: 44 - protein, 15 aa, synthetic construct
MFVFLVLLPLVSSQC

SEQ ID NO: 45 - skipped

SEQ ID NO: 46 - RNA, 11 nt, synthetic construct
  source 1..11: mol_type = other RNA
gggatcctacc

SEQ ID NO: 47-50 - skipped (4 entries)

SEQ ID NO: 51 - protein, 4 aa, synthetic construct
GGGS

(19) United States
(12) Patent Application Publication
(10) Pub. No.: US 2024/0382581 A1
(43) Pub. Date: Nov. 21, 2024

(54) PAN-HUMAN CORONAVIRUS VACCINES

(71) Applicant: ModernaTX, Inc., Cambridge, MA (US)

(72) Inventor: Guillaume Stewart-Jones, Cambridge, MA (US)

(73) Assignee: ModernaTX, Inc., Cambridge, MA (US)

(21) Appl. No.: 18/577,563

(22) PCT Filed: Jul. 8, 2022
(86) PCT No.: PCT/US2022/073577

§ 371 Date: Jan. 8, 2024

Related U.S. Application Data
(60) Provisional application No. 63/322,090, filed on Mar. 21, 2022.
(60) Provisional application No. 63/220,353, filed on Jul. 9, 2021.

(51) Int. Cl. A61K 39/215; A61K 9/127; A61K 9/51; A61K 39/00; A61P 31/14; C12N 7/00

(52) U.S. Cl. CPC A61K 39/215; A61K 9/1271; A61K 9/5123; A61P 31/14; C12N 7/00; A61K 2039/53; A61K 2039/55555; A61K 2039/575; C12N 2770/20034

(57) ABSTRACT

The disclosure relates to pan human coronavirus ribonucleic acid (RNA) vaccines as well as methods of using the vaccines and compositions comprising the vaccines.

### RELATED APPLICATIONS

This application claims the benefit under 35 U.S.C. § 119(e) of U.S. provisional application No. 63/220,353, filed Jul. 9, 2021 and U.S. provisional application No. 63/322,090, filed Mar. 21, 2022, each of which is incorporated by reference herein in its entirety.

### BACKGROUND

Human coronaviruses are highly contagious enveloped, positive single-stranded RNA viruses of the Coronaviridae family. Two sub-families of Coronaviridae are known to cause human disease; the most important being the β-coronaviruses (beta-coronaviruses). The β-coronaviruses are common etiological agents of mild to moderate upper respiratory tract infections. Outbreaks of novel coronavirus infections such as the infections caused by a Wuhan coronavirus, however, have been associated with a high mortality rate death toll. A Severe Acute Respiratory Syndrome Coronavirus 2 (SARSCoV-2) (formerly referred to as a “Wuhan coronavirus,” a “2019 novel coronavirus,” or a “2019-nCoV”) has rapidly infected millions of people worldwide. The pandemic disease that the Wuhan/SARSCoV-2 virus causes has been named by World Health Organization (WHO) as COVID-19 (Coronavirus Disease 2019). The first genome sequence of a SARS-CoV-2 (also referred to as 2019 nCoV) isolate (Wuhan-Hu-1) was sequenced.

### SUMMARY

The disclosure, in some aspects, provides a composition comprising a first messenger ribonucleic acid (mRNA) comprising an open reading frame (ORF) that encodes a first human coronavirus membrane bound spike protein comprising a prefusion conformation stabilizing mutation, a second mRNA comprising an ORF that encodes a second human coronavirus membrane bound spike protein comprising a prefusion conformation stabilizing mutation; and a third mRNA comprising an ORF that encodes a third human coronavirus membrane bound spike protein comprising a prefusion conformation stabilizing mutation, and wherein the first human coronavirus membrane bound spike protein, the second human coronavirus membrane bound spike protein, and the third human coronavirus membrane bound spike protein are from different human coronaviruses, and wherein the mRNAs of are formulated in a lipid nanoparticle.

In some embodiments, the composition further comprises a fourth mRNA comprising an ORF that encodes a fourth human coronavirus membrane bound spike protein comprising a prefusion conformation stabilizing mutation, and wherein the fourth human coronavirus membrane bound spike protein is from a different human coronavirus than the first, second, and third human coronavirus spike proteins, and wherein the mRNA is formulated in the lipid nanoparticle.

In some embodiments, the composition further comprises a fifth mRNA comprising an ORF that encodes a fifth human coronavirus membrane bound spike protein comprising a prefusion conformation stabilizing mutation, and wherein the mRNA is in the lipid nanoparticle.

In some embodiments, each of the human coronavirus membrane bound spike proteins has less than 95% sequence similarity to one another. In some embodiments, each of the human coronavirus membrane bound spike proteins has less than 90% sequence similarity to one another. In some embodiments, each of the human coronavirus membrane bound spike proteins has less than 80% sequence similarity to one another.

In some embodiments, the ratio of the first:second:third mRNA is 1:1:1. In some embodiments, the ratio of the first:second:third:fourth mRNA is 1:1:1:1. In some embodiments, the ratio of the first:second:third:fourth:fifth mRNA is 1:1:1:1:1.

In some embodiments, the prefusion conformation stabilizing mutation is located within a crown of a helix turn region of each human coronavirus membrane bound spike protein, optionally wherein the prefusion conformation stabilizing mutation comprises a double proline mutation or a quadruple glycine (4G) mutation.

In some embodiments, the crown of the helix turn region comprises about 12 amino acids in the S2 subunit between the heptad region 1 (HR1) and central helix (CH) or heptad 2 (HR2) regions of the S2 subunit of the coronavirus spike protein. In some embodiments, the prefusion conformation stabilizing mutation is a substitution relative to a wild-type human coronavirus spike protein.

In some embodiments, the first human coronavirus is selected from the group consisting of: MERS-CoV, SARS-CoV, SARS-CoV-2, NL63, OC43, 229E, and HKU1. In some embodiments, the second human coronavirus is selected from the group consisting of: MERS-CoV, SARS-CoV, SARS-CoV-2, NL63, OC43, 229E, and HKU1. In some embodiments, the third human coronavirus is selected from the group consisting of: MERS-CoV, SARS-CoV, SARS-CoV-2, NL63, OC43, 229E, and HKU1. In some embodiments, the fourth human coronavirus is selected from the group consisting of: MERS-CoV, SARS-CoV, SARS-CoV-2, NL63, OC43, 229E, and HKU1. In some embodiments, the fifth human coronavirus is selected from the group consisting of: MERS-CoV, SARS-CoV, SARS-CoV-2, NL63, OC43, 229E, and HKU1.

In some embodiments, each of the first, second, third, fourth and fifth human coronaviruses is a SARS-CoV-2.

In some embodiments, each of the first, second, third, fourth and fifth human coronaviruses is a selected from the group consisting of: NL63, OC43, 229E, and HKU1. In some embodiments, each mRNA comprises a chemical modification. In some embodiments, each mRNA is chemically modified with 1-methyl-pseudouridine, such that each U in the sequence is a 1-methyl-pseudouridine.

In some embodiments, the lipid nanoparticle comprises a PEG-modified lipid, a non-cationic lipid, a sterol, an ionizable amino lipid, or any combination thereof. In some embodiments, the lipid nanoparticle comprises 0.5-15 mol % PEG-modified lipid; 5-25 mol % non-cationic lipid; 25-55 mol % sterol; and 20-60 mol % ionizable amino lipid. In some embodiments, the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol; and the ionizable amino lipid has the structure of Compound 1:

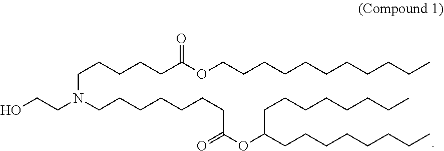 [SMILES: CCCCCCCCCCCOC(=O)CCCCCN(CCO)CCCCCCCC(=O)OC(CCCCCCCC)CCCCCCCC]

In some embodiments, the first human coronavirus is a NL63, the second human coronavirus is a OC43, the third human coronavirus is a 229E, and the fourth human coronavirus is a HKU1.

Aspects of the disclosure provide a composition comprising an mRNA vaccine comprising mRNA encoding at least a first and a second human coronavirus antigen, wherein the human coronavirus antigens comprise at least one human coronavirus spike antigen comprising a prefusion conformation stabilizing mutation of a first circulating human coronavirus, and at least one human coronavirus spike antigen comprising a prefusion conformation stabilizing mutation of a second circulating human coronavirus, wherein the second circulating human coronavirus is a later-emerging variant or strain of the first circulating human coronavirus, wherein the first and second human coronavirus spike antigens have at least one amino acid variance from one another, and wherein the composition further comprises a lipid nanoparticle.

In some embodiments, the human coronavirus antigens are encoded by one or two mRNAs. In some embodiments, the mRNA comprises a single mRNA encoding the at least two human coronavirus antigens. In some embodiments, the mRNA comprises two mRNA each comprising a single open reading frame (ORF) encoding one of the two human coronavirus antigens. In some embodiments, the human coronavirus is selected from the group consisting of: NL63, OC43, 229E, and HKU1.

In some embodiments, the composition further comprises an mRNA comprising an ORF that encodes a third human coronavirus membrane bound spike protein comprising a prefusion conformation stabilizing mutation, wherein the third human coronavirus membrane bound spike proteins has less than 95% sequence similarity to the first and second membrane bound spike proteins, and wherein the mRNA comprising the ORF that encodes the third human coronavirus membrane bound spike protein is in the lipid nanoparticle.

In some embodiments, the composition further comprises an mRNA comprising an ORF that encodes a fourth human coronavirus membrane bound spike protein comprising a prefusion conformation stabilizing mutation, wherein the fourth human coronavirus membrane bound spike proteins has less than 95% sequence similarity to the first and second membrane bound spike proteins, and wherein the mRNA comprising the ORF that encodes the fourth human coronavirus membrane bound spike protein is in the lipid nanoparticle.

In some embodiments, the prefusion conformation stabilizing mutation is located within a crown of a helix turn region of each human coronavirus membrane bound spike protein, optionally wherein the prefusion conformation stabilizing mutation comprises a double proline mutation or a quadruple glycine (4G) mutation. In some embodiments, the crown of the helix turn region comprises about 12 amino acids in the S2 subunit between the heptad region 1 (HR1) and central helix (CH) or heptad 2 (HR2) regions of the S2 subunit of the coronavirus spike protein. In some embodiments, the prefusion conformation stabilizing mutation is a substitution relative to a wild-type human coronavirus spike protein.

In some embodiments, the first, second, third and fourth human coronaviruses are selected from the group consisting of: MERS-CoV, SARS-CoV, SARS-CoV-2, NL63, OC43, 229E, and HKU1.

In some embodiments, the composition is a multivalent mRNA composition and wherein each of the mRNA polynucleotides comprises one or more Identification and Ratio Determination (IDR) sequences. In some embodiments, the IDR sequence comprises between 0 and 25 nucleotides. In some embodiments, the IDR sequence comprises a recognition site for a restriction enzyme. In some embodiments, the restriction enzyme is XbaI.

In some embodiments, each of the mRNA polynucleotides in the composition is complementary with and does not interfere with each other mRNA polynucleotide in the composition.

In some embodiments, the disclosure provides a method comprising administering any one of the compositions described herein to a subject.

An additional aspect of the disclosure provides a method comprising administering to a subject a composition comprising a lipid nanoparticle comprising: (a) a first messenger ribonucleic acid (mRNA) comprising an open reading frame (ORF) that encodes a first human coronavirus membrane bound spike protein comprising a prefusion conformation stabilizing mutation, wherein the first human coronavirus membrane bound spike protein is from a first human coronavirus; (b) a second mRNA comprising an ORF that encodes a second human coronavirus membrane bound spike protein comprising a prefusion conformation stabilizing mutation, wherein the second human coronavirus membrane bound spike protein is from a second human coronavirus; (c) a third mRNA comprising an ORF that encodes a third human coronavirus membrane bound spike protein comprising a prefusion conformation stabilizing mutation, wherein the third human coronavirus membrane bound spike protein is from a third human coronavirus, (d) optionally a fourth mRNA comprising an ORF that encodes a fourth human coronavirus membrane bound spike protein comprising a prefusion conformation stabilizing mutation, wherein the fourth human coronavirus membrane bound spike protein is from a fourth human coronavirus; and (e) optionally a fifth mRNA comprising an ORF that encodes a fifth human coronavirus membrane bound spike protein comprising a prefusion conformation stabilizing mutation, wherein the fifth human coronavirus membrane bound spike protein is from a fifth human coronavirus; wherein the first human coronavirus, the second human coronavirus, and the third human coronavirus and optionally the fourth human coronavirus and the fifth human coronavirus are from different human coronaviruses, and wherein the composition is in an effective amount for producing a neutralizing antibody response against the first human coronavirus, the second human coronavirus, and the third human coronavirus and optionally the fourth human coronavirus and the fifth human coronavirus.

In some embodiments, the composition is in an effective amount for producing a neutralizing antibody response against a human coronavirus that is within a related clade or is a variant of the first human coronavirus. In some embodiments, the composition is in an effective amount for producing a neutralizing antibody response against a human coronavirus that is within a related clade or is a variant of the second human coronavirus. In some embodiments, the composition is in an effective amount for producing a neutralizing antibody response against a human coronavirus that is within a related clade or is a variant of the third human coronavirus. In some embodiments, the composition is in an effective amount for producing a neutralizing antibody response against a human coronavirus that is within a related clade or is a variant of the fourth human coronavirus. In some embodiments, the composition is in an effective amount for producing a neutralizing antibody response against a human coronavirus that is within a related clade or is a variant of the fifth human coronavirus.

In some embodiments, each of the human coronavirus membrane bound spike proteins has less than 95% sequence similarity to one another.

In some embodiments, the mRNAs of (a) and (b) and (c) and (d) and (e) are in a single lipid nanoparticle composition. In some embodiments, the mRNAs of (a) and (b) and (c) and (d) and (e) are in different lipid nanoparticles.

The entire contents of International Application No. PCT/US2016/058327 (Publication No. WO2017/07062) and International Application No. PCT/US2018/022777 (Publication No. WO2018/170347) are incorporated herein by reference.

### BRIEF DESCRIPTION OF THE FIGURES

FIGS. 1A-1H are a series of graphs depicting expression data with polyclonal anti-229E, anti-OC43, anti-NL63, or anti-HKU1 antibodies. FIGS. 1A-ID show the frequency of positive cells for mRNA-1273 (mRNA encoding a SARS-CoV-2 spike protein comprising a double proline stabilizing mutation), OC43 S2P, OC43_S2P_Prko, HKU1_S2P_Prko, NL63_S2P, NL63_S2P_AY567487, NL63_WT_AY567487, 229E_S2P, 229E_WT_AF304460, and Mock (control) following transfection with either 500 ng of mRNA per 1 million cells or 100 ng of mRNA per 1 million cells using anti-229E (FIG. 1A), anti-OC43 (FIG. 1B), anti-NL63 (FIG. 1C), or anti-HKU1 (FIG. 1D) polyclonal antibodies. FIGS. 1E-1H show the mean fluorescence intensity (MFI) of positive cells following treatment with mRNA-1273, OC43 S2P, OC43_S2P_Prko, HKU1_S2P_Prko, NL63_S2P, NL63_S2P_AY567487, NL63_WT_AY567487, 229E_S2P, and 229E_WT_AF304460 over the MFI of Mock cells following transfection with either 500 ng mRNA per 1 million cells or 100 ng mRNA per 1 million cells measured using anti-229E (FIG. 1E), anti-OC43 (FIG. 1F), anti-NL63 (FIG. 1G), or anti-HKU1 (FIG. 1H) antibodies.

FIGS. 2A-2J are a series of graphs depicting the expression data with polyclonal antibodies from FIGS. 1A-1H and additional monoclonal antibodies CC9.3, CC9.2, B6, CC40.8, 28D9, and 1.6C7. The graphs show frequency of positive cells in EXPI293 cells for mRNA-1273, OC43_SP_2P_1273-TM, OC43_S2P_Prko, HKU1_S2P_Prko, 229E_S2P, 229E_WT_AF304460, NL63_S2P, NL63_S2P_AY567487, NL63_WT_AY567487 and Mock (control) 48 hours post-transfection with either 500 ng of mRNA per 1 million cells or 100 ng of mRNA per 1 million cells.

FIGS. 3A-3J are a series of graphs showing the relative expression of mRNA-1273, OC43_SP_2P_1273-TM, OC43_S2P_Prko, HKU1_S2P_Prko, 229E_S2P, 229E_WT_AF304460, NL63_S2P, NL63_S2P_AY567487, NL63_WT_AY567487 mRNA as the mean fluorescence intensity (MFI)×frequency of positive EXPI293 cells normalized to mock expression at 48 hours post transfection with either 500 ng of mRNA per 1 million cells or 100 ng of mRNA per 1 million cells.

FIGS. 4A-4D are a series of graphs showing the MFI for mRNA-1273, OC43_SP_2P_1273-TM, OC43_S2P_Prko, HKU1_S2P_Prko, 229E_S2P, 229E_WT_AF304460, NL63_S2P, NL63_S2P_AY567487, NL63_WT_AY567487, and Mock 48 hours post transfection with either 500 ng of mRNA per 1 million cells or 100 ng of mRNA per 1 million cells. Results were normalized to the mock group.

FIGS. 5A-5D show the binding to S-2P antigens from OC43 S-2P (FIG. 5A), HKU1 S-2P (FIG. 5B), 229E S-2P (FIG. 5C), and NL63 S-2P (FIG. 5D) viruses via mean fluorescence intensity (MFI) across OC43_S2P_Prko, HKU1_S2P_Prko, 229E_S2P, and NL63_S2P immunogen groups. The control group is represented by PBS.

FIGS. 6A-6G show the binding to S antigens from OC43 (FIGS. 6A, 6E), HUK1 (FIG. 6B), 229E (FIGS. 6C, 6F), and NL63 (FIGS. 6D, 6G) viruses via mean MFI across OC43_S2P_Prko, HKU1_S2P_Prko, 229E_S2P, and NL63_S2P immunogen groups. The control group is represented by PBS.

FIGS. 7A-7D show the binding to S1 viral antigens from OC43 (FIG. 7A), HUK1 (FIG. 7B), 229E (FIG. 7C), and NL63 (FIG. 7D) via mean MFI across OC43_S2P_Prko, HKU1_S2P_Prko, 229E_S2P, and NL63_S2P immunogen groups. The control group is represented by PBS.

FIGS. 8A-8E show the binding to S2P (FIG. 8A), RBD (FIG. 8B), NTD (FIG. 8C), S (FIG. 8D), and S2 (FIG. 8E) viral antigens from OC43 virus via mean MFI across OC43_S2P_Prko, HKU1_S2P_Prko, 229E_S2P, and NL63_S2P combination mRNA treatment.

FIGS. 9A-9E show the binding to S2P (FIG. 9A), RBD (FIG. 9B), NTD (FIG. 9C), S (FIG. 9D), and S2 (FIG. 9E) viral antigens from HKU1 virus via mean MFI across OC43_S2P_Prko, HKU1_S2P_Prko, 229E_S2P, and NL63_S2P combination mRNA treatment.

FIGS. 10A-10C show the binding to S2P (FIG. 9A), NTD (FIG. 9B), and S (FIG. 9C) viral antigens from HKU1 virus via mean MFI across OC43_S2P_Prko, HKU1_S2P_Prko, 229E_S2P, and NL63_S2P combination mRNA treatment. FIGS.

11A-11D show the binding to S2P (FIG. 11A), RBD (FIG. 11B), NTD (FIG. 11C) and S (FIG. 11D) viral antigens from NL63 virus via mean MFI across OC43_S2P_Prko, HKU1_S2P_Prko, 229E_S2P, and NL63_S2P combination mRNA treatment.

FIGS. 12A-12F show the binding to S2P (FIGS. 12A & 12D), RBD (FIGS. 12B & 12E), and NTD (FIGS. 12C & 12F) viral antigens from WT CoV2 (FIGS. 12A-12C) and omicron (FIGS. 12D-12F) viruses via mean MFI across OC43_S2P_Prko, HKU1_S2P_Prko, 229E_S2P, and NL63_S2P combination mRNA treatment.

FIG. 13 is a schematic showing an embodiment of a structure described herein (“TM domain” represents a transmembrane domain).

### DETAILED DESCRIPTION

The present disclosure provides compositions (e.g., immunizing/immunogenic compositions such as mRNA vaccines) that elicit potent neutralizing antibodies against multiple human coronavirus antigens, providing pan protection resulting from an immune response developed by exposure to a radial burst of unique antigen genotypes within the vaccine. In some embodiments, a single vaccine can provide immune protection against a wide spectrum of coronaviruses, including viruses that may have been in circulation for years, viruses currently circulating and even viruses that have not yet been discovered. Most vaccines include one or more antigens that are reflected or believed to be reflected in currently circulating viruses. The vaccine is designed to attempt to provoke an immune response specifically against the circulating virus so that individual who receives the vaccine mounts a specific immune response to recognize and destroy the virus. Even if a vaccine can successfully generate antibodies against a circulating virus, the vaccine may be rendered less effective or useless if the virus mutates; the antibodies may not recognize the viral mutant as well as the parent strain.

The instant disclosure provides a pan human coronavirus vaccine that has broad neutralizing capabilities against different circulating coronaviruses as well as older coronaviruses and coronaviruses that have future pandemic potential. The pan human coronavirus in some embodiments comprises a composition of LNP and multiple mRNA (e.g., 3, 4, 5, 6, 7, 8, 9, 10) each encoding a human coronavirus membrane bound spike protein comprising a double proline stabilizing mutation from a different coronavirus. In some embodiments the different mRNAs are in different LNPs. In other embodiments the different mRNAs are formulated in the same LNP.

In some embodiments the vaccine composition can produce a neutralizing antibody response against the first human coronavirus, the second human coronavirus, and the third human coronavirus and optionally the fourth human coronavirus and the fifth human coronavirus. In some embodiments the composition can produce a neutralizing antibody response against a human coronavirus that is a clade or variant of any or all of the human coronaviruses in which an antigen is included in the vaccine.

In some embodiments, the coronavirus is human coronavirus OC43 (HCoV-OC43). HCoV-OC43 is a betacoronavirus that infects humans and cattle. The virus is an enveloped, positive-sense, single stranded RNA virus, and is a virus responsible for the common cold. It is thought to have originated in rodents, and then passed through cattle as intermediate hosts. There are four genotypes (A-D), and recombinant genotype D variants are the most recent, dating back to 2004 (Lim et al., Diseases. 4(3): 26). The virus' genome is approximately 30 kb in length, comprising 11 major open reading frames (ORFs) encoding the envelope (E) protein, spike (S) protein, nucleocapsid (N) protein, and the (M) membrane protein, among others (J. Virol. 2005; 79(3): 1595-1604).

HKU1-CoV is a betacoronavirus that infects the human respiratory tract, causing symptoms of the common cold, with the potential to progress to more severe pneumonia and/or bronchitis. HKU1-CoV likely originated from spillover of a rodent betacoronavirus into humans, but is now endemic in origin. See, e.g., Lau et al. J Clin Microbiol. 2006. 44(6):2063-2071. The HKU1-CoV is also about 30 kb long, and includes M, E, N, and S proteins (Ency. Of Virol. 2021:428-440).

In some embodiments, the coronavirus may be NL63-CoV, an alphacoronavirus that infects the human respiratory tract, particularly of children. Infection by NL63-CoV typically causes upper respiratory tract symptoms similar to the common cold, but may progress to more severe symptoms of pneumonia or bronchitis following infection of the lower respiratory tract (Abdul-Rasool et al. Open Virol J. 2010. 4:76-84). The HKU1-CoV is also about 30 kb long, and includes M, E, N, and S proteins (Ency. Of Virol. 2021:428-440).

In another embodiment, the coronavirus may be human coronavirus 229E, which infects humans and bats. It is an enveloped, positive-sense, single-stranded RNA virus. It is one of the viruses responsible for the common cold. Its genome is approximately 30,000 nucleotides long and includes spike, envelope, membrane, and nucleocapsid proteins (Korsman et al., Virology, 2012). Human coronavirus 229E is approximately 30 kb long, and includes M, E, N, and S proteins (Ency. Of Virol. 2021:428-440).

In another embodiment, the coronavirus may be a human SARS coronavirus. SARS-CoV, for severe acute respiratory distress syndrome (SARS), is a betacoronavirus that infects the human respiratory tract. Symptoms of SARS-CoV infection can range from mild, such as those similar to a common cold (nasal discharge, sore throat, low fever), to severe, including acute respiratory distress syndrome, which may be fatal. See, e.g. Nat Rev Microbiol. 2009. 7(3):226-236.

In another embodiment, the coronavirus may be human MERS coronavirus. MERS-CoV, for Middle East Respiratory Syndrome (MERS), is a betacoronavirus that infects the human respiratory tract. Symptoms of MERS-CoV infection can range from mild, such as those similar to a common cold (nasal discharge, sore throat, low fever), to severe, including acute respiratory distress syndrome, which may be fatal. MERS-CoV is endemic in dromedary camels of East Africa and the Arabian Peninsula, and was first reported to infect humans in 2012. Though zoonotic in origin, human-to-human transmission is possible. See, e.g., Ramadan et al. Germs. 2019. 9(1):35-42.

In some embodiments, a composition comprises RNA (e.g., messenger RNA (mRNA)) encoding a human coronavirus antigen, such as a NL63, OC43, 229E or HKU1 antigen. In some embodiments, a composition comprises two RNAs (e.g., mRNA), each encoding a human coronavirus antigen, such as a NL63, OC43, 229E or HKU1 antigen. In some embodiments, a composition comprises three RNAs (e.g., mRNA), each encoding a human coronavirus antigen, such as a NL63, OC43, 229E or HKU1 antigen. In some embodiments, a composition comprises four RNAs (e.g., mRNA), each encoding a human coronavirus antigen, such as a NL63, OC43, 229E or HKU1 antigen. In some embodiments, the human coronavirus antigen is a spike protein. In some embodiments, the human coronavirus antigen is a stabilized prefusion spike protein.

In some embodiments, a composition comprises RNA (e.g., messenger RNA (mRNA)) encoding a human coronavirus antigen, such as a SARS-CoV antigen (i.e., SARS CoV-1 or SARS-Cov-2) or MERS. In some embodiments, a composition comprises two RNAs (e.g., mRNA), each encoding a human coronavirus antigen, such as a SARS-CoV or MERS antigen. In some embodiments, a composition comprises three RNAs (e.g., mRNA), each encoding a human coronavirus antigen, such as a SARS-CoV or MERS antigen. In some embodiments, a composition comprises four RNAs (e.g., mRNA), each encoding a human coronavirus antigen, such as a SARS-CoV or MERS antigen. In some embodiments, the human coronavirus antigen is a spike protein. In some embodiments, the human coronavirus antigen is a stabilized prefusion spike protein.

In some embodiments the composition does not comprise an mRNA encoding a SARS-CoV or MERS antigen.

In some embodiments, a composition comprises at least three RNAs (e.g., messenger RNA (mRNA)), each encoding a human coronavirus antigen, such that the composition includes at least one antigen from NL63, at least one antigen from OC43, and at least one antigen from 229E. In some embodiments, a composition comprises at least three RNAs (e.g., mRNA), each encoding a human coronavirus antigen, such that the composition includes at least one antigen from NL63, at least one antigen from OC43, and at least one antigen from or HKU1. In some embodiments, a composition comprises at least three RNAs (e.g., mRNA), each encoding a human coronavirus antigen, such that the composition includes at least one antigen from 229E, at least one antigen from OC43, and at least one antigen from or HKU1. In some embodiments, a composition comprises at least three RNAs (e.g., mRNA), each encoding a human coronavirus antigen, such that the composition includes at least one antigen from NL63, at least one antigen from 229E, and at least one antigen from or HKU1.

In some embodiments, a composition comprises at least three RNAs (e.g., mRNA), each encoding a human coronavirus antigen, such that the composition includes at least one antigen from SARS-CoV, at least one antigen from OC43, and at least one antigen from or HKU1. In some embodiments, a composition comprises at least three RNAs (e.g., mRNA), each encoding a human coronavirus antigen, such that the composition includes at least one antigen from SARS-CoV, at least one antigen from OC43, and at least one antigen from or 229E. In some embodiments, a composition comprises at least three RNAs (e.g., mRNA), each encoding a human coronavirus antigen, such that the composition includes at least one antigen from SARS-CoV, at least one antigen from OC43, and at least one antigen from or NL63. In some embodiments, a composition comprises at least three RNAs (e.g., mRNA), each encoding a human coronavirus antigen, such that the composition includes at least one antigen from SARS-CoV, at least one antigen from NL63, and at least one antigen from or HKU1. In some embodiments, a composition comprises at least three RNAs (e.g., mRNA), each encoding a human coronavirus antigen, such that the composition includes at least one antigen from SARS-CoV, at least one antigen from NL63, and at least one antigen from or 229E.

In yet other embodiments, a composition comprising mRNA encoding at least first and second human coronavirus antigens, wherein the human coronavirus antigens comprise at least one human coronavirus spike antigen comprising a double proline stabilizing mutation of a first circulating human coronavirus, and at least one human coronavirus spike antigen comprising a double proline stabilizing mutation of a second circulating human coronavirus, wherein the second circulating human coronavirus is a variant or strain of the first circulating human coronavirus. In some embodiments the first and second circulating human coronaviruses are SARS-CoV2. In some embodiments, the first circulating human coronavirus is an earlier circulating strain during a pandemic, and the second circulating strain is a later evolved or later emerging variant of the same pandemic coronavirus. In some embodiments the first and second circulating human coronaviruses are not SARS-CoV2, particularly when the composition does not include additional antigens.

The composition includes an mRNA encoding an antigen from at least one parent or original coronavirus, an mRNA encoding an antigen from a strain or variant of a parent or original coronavirus and optionally one or more mRNAs encoding antigens, each antigen from a different coronavirus.

Without wishing to be bound by theory, it is thought that the vaccine compositions described herein (e.g., vaccine compositions comprising mRNA or mRNAs encoding at least two human coronavirus spike proteins, each comprising a stabilizing double proline mutation) induce a strong immune response against each of the antigens present in the composition, thus producing an effective and potent mRNA vaccine. Administration of the mRNA encoding spike (S) protein antigens, such as the various variants of the prefusion stabilized spike protein antigens described herein, results in delivery of the mRNA to immune tissues and cells of the immune system where it is rapidly translated into protein antigens. Other immune cells, for example, B cells and T cells, are then able to develop an immune response against the encoded protein and ultimately create a long-lasting protective response against the selected human coronavirus. Low immunogenicity, a drawback in protein vaccine development due to poor presentation to the immune system or incorrect folding of the antigens, is avoided by using the highly effective mRNA vaccines encoding spike protein as disclosed herein. Therefore, the vaccines described herein may be used to induce a balanced immune response, comprising both cellular and humoral immunity. Such a vaccine can be administered to seropositive or seronegative subjects. For example, a subject may be naïve and not have antibodies that react with coronavirus antigenic polypeptides of the vaccine, or may have preexisting antibodies to coronavirus (e.g., NL63, OC43, 229E and/or HKU1) antigenic polypeptides of the vaccine because they have previously had an infection with the coronavirus or may have previously been administered a dose of a vaccine (e.g., an mRNA vaccine) that induces antibodies against the coronavirus (e.g., NL63, OC43, 229E and/or HKU1). In some embodiments, the subject may be seronegative for 1, 2, 3, or all 4 of the human coronavirus antigens encoded by the mRNAs in the vaccine composition. In some embodiments, the subject may be seropositive for 1, 2, 3, or all 4 of the human coronavirus antigens encoded by the mRNAs in the composition.

### Antigens

Antigens, as used herein, are proteins capable of inducing an immune response (e.g., causing an immune system to produce antibodies against the antigens). Herein, use of the term “antigen” encompasses immunogenic proteins and immunogenic fragments (an immunogenic fragment that induces (or is capable of inducing) an immune response to a (at least one) coronavirus), unless otherwise stated. It should be understood that the term “protein” encompasses peptides and the term “antigen” encompasses antigenic fragments. Other molecules may be antigenic such as bacterial polysaccharides or combinations of protein and polysaccharide structures, but for the viral vaccines included herein, viral proteins, fragments of viral proteins and designed and or mutated proteins derived from human coronaviruses (e.g., NL63, OC43, 229E or HKU1) are the antigens provided herein.

The envelope spike (S) proteins of coronaviruses determine the virus host tropism and entry into host cells. Coronavirus spike (S) protein is a choice antigen for the vaccine design as it can induce neutralizing antibodies and protective immunity. S protein is critical for coronavirus infections. The organization of the S protein is similar among coronaviruses, such as SARS-CoV-2, SARS-CoV, MERS-CoV, HKU1-CoV, OC43-CoV, 229-CoV, and NL63-CoV.

As used herein, the term “Spike protein” refers to a glycoprotein that that forms homotrimers protruding from the envelope (viral surface) of viruses including betacoronaviruses. Trimerized Spike protein facilitates entry of the virion into a host cell by binding to a receptor on the surface of a host cell followed by fusion of the viral and host cell membranes. The S protein is a highly glycosylated and large type I transmembrane fusion protein that is made up of 1,160 to 1,400 amino acids, depending upon the type of virus. mRNAs of the invention are designed to produce multiple different coronavirus Spike proteins (i.e., encode Spike proteins such that Spike protein is expressed when the mRNA is delivered to a cell or tissue, for example a cell or tissue in a subject), as well as antigenic variants thereof. The skilled artisan will understand that, while an essentially full length or complete Spike protein may be necessary for a virus, e.g., a betacoronavirus, to perform its intended function of facilitating virus entry into a host cell, a certain amount of variation in Spike protein structure and/or sequence is tolerated when seeking primarily to elicit an immune response against Spike protein. For example, minor truncation, e.g., of one to a few, possibly up to 5 or up to 10 amino acids from the N- or C-terminus of the encoded Spike protein, e.g., encoded Spike protein antigen, may be tolerated without changing the antigenic properties of the protein. Likewise, variation (e.g., conservative substitution) of one to a few, possibly up to 5 or up to 10 amino acids (or more) of the encoded Spike protein, e.g., encoded Spike protein antigen, may be tolerated without changing the antigenic properties of the protein. Where minor variations are made in encoded Spike protein sequences, the variant preferably has the same activity as the reference Spike protein sequence and/or has the same immune specificity as the reference Spike protein, as determined for example, in immunoassays (e.g., enzyme-linked immunosorbent assays (ELISA assays).

In some embodiments, the modified proteins of the SARS-CoV-2 S protein and other CoV S proteins provided herein, (e.g., comprising one more glycine and/or proline substitutions, for example L984G, D985G, K986G, and/or V987G), are more flexible than their parental SARS-CoV-2 or other CoV S proteins. The enhanced flexibility of SARS-CoV-2 and other CoV 2 proteins is thought to elicit antibody responses to a broader range of S protein epitopes, further improve vaccine efficacy at the same dose, and/or maintain efficacy at lower doses.

Exemplary sequences of the human coronavirus antigens and the RNA (e.g., mRNA) encoding the human coronavirus antigens of the compositions of the present disclosure are provided in Table 1.

In some embodiments, a composition comprises an RNA (e.g., mRNA) that encodes a coronavirus antigen that comprises an amino acid sequence having at least 80%, at least 85%, at least 90%, at least 95%, at least 98%, or 100% identity to the amino acid sequence of any one of SEQ ID NOs: 5, 8, 11, or 14. In some embodiments, a composition comprises an RNA (e.g., mRNA) that encodes a coronavirus antigen that comprises the sequence of SEQ ID NO: 5. In some embodiments, a composition comprises an RNA (e.g., mRNA) that encodes a coronavirus antigen that comprises the sequence of SEQ ID NO: 8. In some embodiments, a composition comprises an RNA (e.g., mRNA) that encodes a coronavirus antigen that comprises the sequence of SEQ ID NO: 11. In some embodiments, a composition comprises an RNA (e.g., mRNA) that encodes a coronavirus antigen that comprises the sequence of SEQ ID NO: 14.

It should be understood that any one of the antigens encoded by the RNA described herein may or may not comprise a signal sequence.

The compositions of the present disclosure comprise a (at least one) RNA having an open reading frame (ORF) encoding a coronavirus antigen. In some embodiments, the compositions of the present disclosure comprise 1, 2, 3, 4, 5, 6, 7, 8, or more RNAs, each having an ORF encoding a different coronavirus antigen. In some embodiments, the RNA is a messenger RNA (mRNA). In some embodiments, the RNA (e.g., mRNA) further comprises a 5′ UTR, 3′ UTR, a poly(A) tail and/or a 5′ cap analog.

It should also be understood that the coronavirus vaccine of the present disclosure may include any 5′ untranslated region (UTR) and/or any 3′ UTR. Exemplary UTR sequences are provided in the Sequence Listing (e.g., SEQ ID NOs: 2, 36, 4, or 37); however, other UTR sequences may be used or exchanged for any of the UTR sequences described herein. UTRs may also be omitted from the RNA polynucleotides provided herein.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

In some embodiments, a composition comprises an RNA (e.g., mRNA) that comprises a nucleotide sequence having at least 80%, at least 85%, at least 90%, at least 95%, at least 98%, or 100% identity to the nucleotide sequence of any one of SEQ ID NOs: 1, 6, 9, or 12.

In some embodiments, a composition comprises an RNA (e.g., mRNA) that comprises an ORF that comprises a nucleotide sequence having at least 80%, at least 85%, at least 90%, at least 95%, at least 98%, or 100% identity to the nucleotide sequence of any one of SEQ ID NOs: 3, 7, 10, or 13.

### Modified Antigens

In some embodiments, the compositions of the present disclosure include RNA that encodes a modified coronavirus antigen. Modified coronavirus antigens may include any synthetic modifications such as the addition of 2 prolines to stabilize the pre-fusion spike antigen structure or naturally occurring modifications such as those found in variant strains or clades. Modified antigens refer to molecules that differ in their amino acid sequence from a wild-type, native, or reference sequence. The modified antigen/polypeptide may possess substitutions, deletions, and/or insertions at certain positions within the amino acid sequence, as compared to a native or reference sequence. Ordinarily, modified antigens possess at least 50% identity to a wild-type, native or reference sequence. In some embodiments, modified antigens share at least 80%, or at least 90% identity with a wild-type, native, or reference sequence.

Modified antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Modified antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein can be measured using art recognized techniques, e.g., by determining expression of antigens in a vaccinated subject over time and/or by looking at the durability of the induced immune response. The stability of protein(s) encoded by a modified nucleic acid may be measured by assaying thermal stability or stability upon urea denaturation or may be measured using in silico prediction. Methods for such experiments and in silico determinations are known in the art.

Some modified antigens are found in viruses that have evolved naturally relative to a parent virus. Errors in viral RNA with respect to a parent or original virus are known as mutations. Viruses with these mutations are called “variants”. Variants may differ by a single or many mutations relative to the parent or original virus, regardless of whether those mutations cause any change in phenotype or characteristics of the virus. A variant is referred to as a strain when it shows one or more distinct physical or functional properties from the parent virus. Thus, a “strain” is a variant that has structural differences that result in functional differences relative to its parent virus. A “clade”, as used herein is similar to a variant and is used to describe a virus having a genetic change or changes sufficient to form a new group of viruses, all derived from a common ancestor.

In some embodiments, a composition comprises an RNA or an RNA ORF that comprises a nucleotide sequence of any one of the sequences provided herein (see, e.g., Sequence Listing and Table 1), or comprises a nucleotide sequence at least 80%, at least 85%, at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an RNA (e.g., mRNA) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, an RNA (e.g., mRNA) includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an RNA (e.g., mRNA) does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An RNA (e.g., mRNA) may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an RNA (e.g., mRNA) has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, a composition comprises an RNA (e.g., mRNA) having an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure. In some embodiments, the signal peptide may comprise one of the following sequences: MDSKGSSQKGSRLLLLLVVSNLLLPQGVVG (SEQ ID NO: 38), MDWTWILFLVAAATRVHS (SEQ ID NO: 39); METPAQLLFLLLLWLPDTTG (SEQ ID NO: 40); MLGSNSGQRVVFTILLLLVAPAYS (SEQ ID NO: 41); MKCLLYLAFLFIGVNCA (SEQ ID NO: 42); MWLVSLAIVTACAGA (SEQ ID NO: 43); or MFVFLVLLPLVSSQC (SEQ ID NO: 44).

### Fusion Proteins

In some embodiments, a composition of the present disclosure includes an RNA (e.g., mRNA) encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the coronavirus antigen. Antigenic fusion proteins, in some embodiments, retain the functional property from each original protein.

### Scaffold Moieties

The RNA (e.g., mRNA) vaccines as provided herein, in some embodiments, encode fusion proteins that comprise coronavirus antigens linked to scaffold moieties. In some embodiments, such scaffold moieties impart desired properties to an antigen encoded by a nucleic acid of the disclosure. For example scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of −22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) that self-assembles into particles of 24-31 nm diameter, which resembled the viral cores obtained from HBV-infected human liver. HBcAg self-assembles into two classes of differently sized nanoparticles of 300 Å and 360 Å diameter, corresponding to 180 or 240 protomers. In some embodiments, the coronavirus antigen is fused to HBsAG or HBcAG to facilitate self-assembly of nanoparticles displaying the coronavirus antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, an RNA of the present disclosure encodes a coronavirus antigen (e.g., SARS-CoV-2 S protein) fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS (SEQ ID NO: 51) linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally-occurring or wild-type sequence (e.g., a naturally-occurring or wild-type mRNA sequence encoding a coronavirus antigen). In some embodiments, a codon optimized sequence shares less than 85% sequence identity to a naturally-occurring or wild-type sequence (e.g., a naturally-occurring or wild-type mRNA sequence encoding a coronavirus antigen). In some embodiments, a codon optimized sequence shares less than 80% sequence identity to a naturally-occurring or wild-type sequence (e.g., a naturally-occurring or wild-type mRNA sequence encoding a coronavirus antigen). In some embodiments, a codon optimized sequence shares less than 75% sequence identity to a naturally-occurring or wild-type sequence (e.g., a naturally-occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a coronavirus antigen). In some embodiments, a codon optimized sequence shares between 65% and 75% or about 80% sequence identity to a naturally-occurring or wild-type sequence (e.g., a naturally-occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an RNA (e.g., mRNA) is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/036773; PCT/US2015/036759; PCT/US2015/036771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 46) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-0) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 2 and SEQ ID NO: 36.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

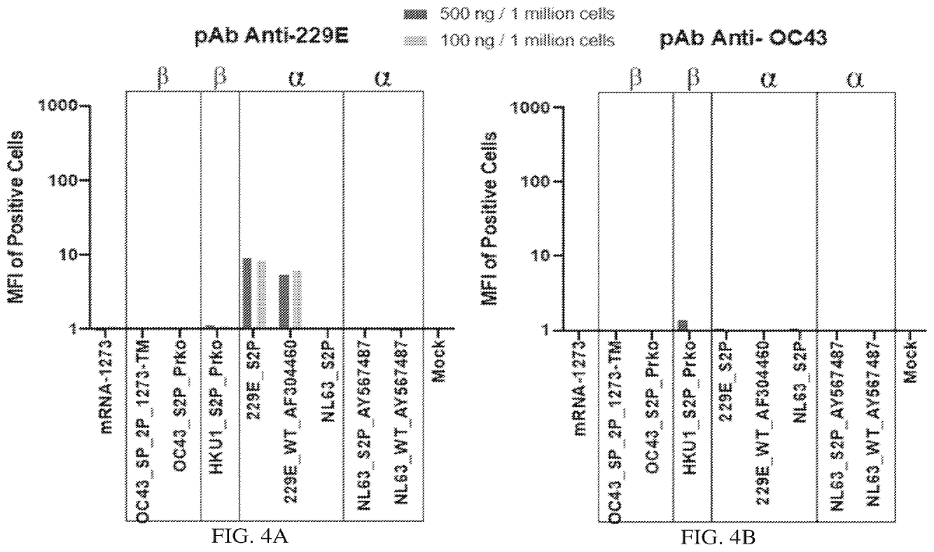

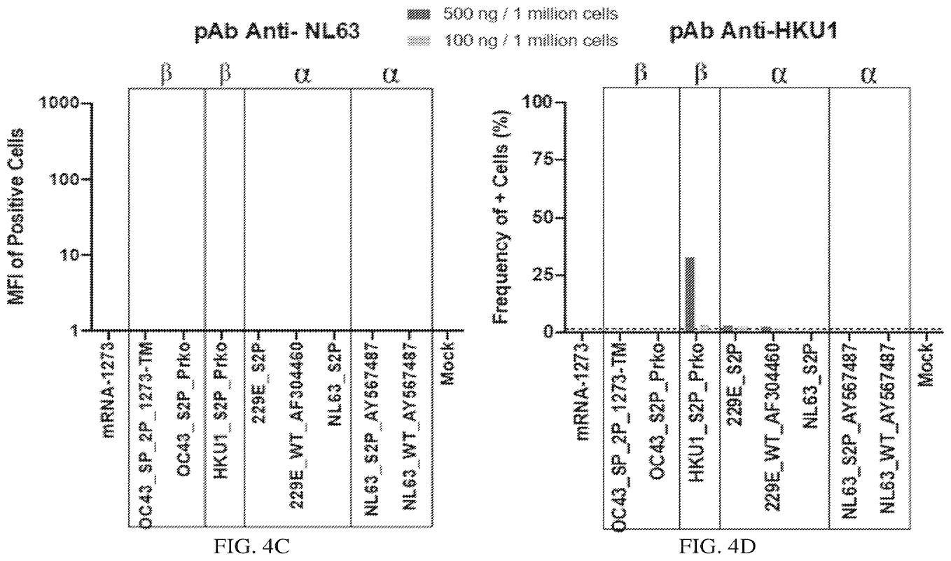

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A modified β-globin construct with enhanced stability in some cell types by cloning two sequential human β-globin 3′UTRs head to tail has been developed and is well known in the art (US2012/0195936, WO2014/071963). In addition a2-globin, a1-globin, UTRs and mutants thereof are also known in the art (WO2015101415, WO2015024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit β globin and hepatitis B virus (HBV), α-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′UTR (WO2015101414), FIG. 4 (WO2015101415), and human albumin 7 (WO2015101415).

In some embodiments, a 3′ UTR of the present disclosure comprises a sequence selected from SEQ ID NO: 4 and SEQ ID NO: 37.

Those of ordinary skill in the art will understand that 5′UTRs that are heterologous or synthetic may be used with any desired 3′ UTR sequence. For example, a heterologous 5′UTR may be used with a synthetic 3′UTR or a synthetic 5′ UTR may be used with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In general, a DNA-dependent RNA polymerase transcribes a DNA template containing an appropriate promoter into an RNA transcript. The poly(A) tail can be generated co-transcriptionally by incorporating a poly(T) tract in the template DNA or separately by using a poly(A) polymerase. Eukaryotic mRNAs start with a 5′ cap (e.g., a 5′ m7GpppX cap). Typically, the 5′ cap begins with an inverted G with N7Me (required for eIF4E binding). A preferred cap, Cap1 contains 2′OMe at the +1 position) followed by any nucleoside at +2 position. This cap can be installed post-transcriptionally, e.g., enzymatically (after transcription) or co-transcriptionally (during transcription).

Post-transcriptional capping can be carried out using the vaccinia capping enzyme, and allows for complete capping of the RNA, generating a cap 0 structure on RNA carrying a 5′ terminal triphosphate or diphosphate group, the cap 0 structure being required for efficient translation of the mRNA in vivo. The cap 0 structure can then be further modified into cap 1 using a cap-specific 2′O methyltransferase. Vaccinia capping enzyme and 2′O methyltransferase have been used to generate cap 0 and cap 1 structures on in vitro transcripts, for example, for use in transfecting eukaryotic cells or in mRNA therapeutic applications to drive protein synthesis. While post-transcriptional capping by vaccinia capping enzymes can yield either Cap 0 or Cap 1 structures, it is an expensive process when utilized for large-scale mRNA production, for example, vaccinia is costly and in limited supply and there can be difficulties in purifying an IVT mRNA (e.g., removing S-adenosylmethionine (SAM) and 2′O-methyltransferase). Moreover, capping can be incomplete due to inaccessibility of structured 5′ ends.

Co-transcriptional capping using a cap analog has certain advantages over vaccinia capping, for example, the process requires a simpler workflow (i.e., no need for a purification step between transcription and capping.) Traditional co-transcriptional capping methods utilize the dinucleotide ARCA (anti-reverse cap analog) and yield Cap 0 structures. ARCA capping has drawbacks, however, for example, the resulting Cap 0 structures can be immunogenic, and the process often results in low yields and/or poorly capped material. Another potential drawback of this approach is a theoretical capping efficiency of <100%, due to competition from the GTP for the starting nucleotide. For example, co-transcriptonal capping using ARCA typically requires a 10:1 ratio of ARCA:GTP to achieve >90% capping (needed to outcompete GTP for initiation).

The present disclosure provides trinucleotide mRNA cap analogs and methods of using them in co-transcriptional capping methods (e.g., featuring T7 RNA polymerase) for the in vitro synthesis of mRNA. Use of a trinucleotide cap analog may provide a solution to several of the above-described problems associated with vaccinia or ARCA capping. In addition, these methods provide flexibility in modifying the penultimate nucleobase which may alter binding behavior, or affect the affinity of these caps towards decapping enzymes, or both, thus potentially improving stability of the respective mRNA. An exemplary trinucleotide for use in the herein-described co-transcriptional capping methods is the m7GpppAG (GAG) trinucleotide. Use of this trinucleotide results in the nucleotide at the +1 position being A instead of G. Both +1G and +1A are caps that can be found in naturally-occurring mRNAs.

T7 RNA polymerase prefers to initiate with 5′ GTP. Accordingly, most conventional mRNA transcripts start with 5′-GGG (based on transcription from a T7 promoter sequence such as 5′TAATACGACTCACTATAGGGNNNNNNNNN . . . 3′ (SEQ ID NO: 30) (TATA being referred to as the “TATA box”). T7 RNA polymerase typically transcribes DNA downstream of a T7 promoter (5′ TAATACGACTCACTATAG 3′ (SEQ ID NO: 31), referencing the coding strand). T7 polymerase starts transcription at the underlined G in the promoter sequence. The polymerase then transcribes using the opposite strand as a template from 5′→3′. The first base in the transcript will be a G.

The herein-described processes capitalize on the fact that the T7 enzyme has limited initiation activity with the single nucleotide ATP, driving T7 to initiate with the trinucleotide rather than ATP. The process thus generates an mRNA product with >90% functional cap post-transcription. The process is an efficient “one-pot” mRNA production method that includes, for example, the GAG trinuclotide (GpppAG; mGpppAmG) in equimolar concentration with the NTPs, GTP, ATP, CTP and UTP. The process features an “A-start” DNA template that initiates transcription with 5′ adenosine (A). As defined herein, “A-start” and “G-start” DNA templates are double-stranded DNA having requisite nucleosides in the template strand, such that the coding strand (and corresponding mRNA) begin with A or G, respectively. For example, a G-start DNA template features a template strand having the nucleobases CC complementary to GG immediately downstream of the TATA box in the T7 promoter (referencing the coding strand), and an A-start DNA template features a template strand having the nucleobases TC complementary to the AG immediately downstream of the TATA box in the T7 promoter (referencing the coding strand).

An exemplary T7 promoter sequence featured in an A-start DNA template of the disclosure is depicted here:

(SEQ ID NO: 32)
5′TAATACGACTCACTATAAGNNNNNNNNNN . . . 3′
(SEQ ID NO: 33)
3′ATTATGCTGAGTGATATTCNNNNNNNNNN . . . 3′

The trinucleotide-based capping methods described herein provide flexibility in dictating the penultimate nucleobase. The trinucleotide capping methods of the disclosure provide efficient production of capped mRNA, for example, 95-98% capped mRNA with a natural cap 1 structure.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants thereof may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7 mG(5′)ppp(5′)NlmpNp.

### Identification and Ratio Determination (IDR) Sequences

An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the RNA (e.g., mRNA) of the disclosure is formulated in a lipid nanoparticle (LNP). Lipid nanoparticles typically comprise ionizable amino (cationic) lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticles. The vaccines can be made, for example, using mixing processes such as microfluidics and T-junction mixing of two fluid streams, one of which contains the mRNA and the other has the lipid components. In some embodiments, the vaccines are prepared by combining an ionizable amino lipid, a phospholipid (such as DOPE or DSPC), a PEG lipid (such as 1,2-dimyristoyl-OT-glycerol methoxypoly ethylene glycol, also known as PEG-DMG), and a structural lipid (such as cholesterol) in an alcohol (e.g., ethanol). The lipids may be combined to yield desired molar ratios and diluted with water and alcohol (e.g., ethanol) to a final lipid concentration of between about 5.5 mM and about 25 mM, for example.

Vaccines including mRNA and a lipid component may be prepared, for example, by combining a lipid solution with an mRNA solution at lipid component to mRNA wt:wt ratios of between about 5:1 and about 50:1. The lipid solution may be rapidly injected using a microfluidic based system (e.g., NanoAssemblr) at flow rates between about 10 ml/min and about 18 ml/min, for example, into the mRNA solution to produce a suspension (e.g., with a water to alcohol ratio between about 1:1 and about 4:1).

Vaccines can be processed by dialysis to remove the alcohol (e.g., ethanol) and achieve buffer exchange. Formulations may be dialyzed against phosphate buffered saline (PBS), pH 7.4, for example, at volumes greater than that of the primary product (e.g., using Slide-A-Lyzer cassettes (Thermo Fisher Scientific Inc., Rockford, IL)) with a molecular weight cutoff of 10 kD, for example. The forgoing exemplary method induces nanoprecipitation and particle formation. Alternative processes including, but not limited to, T-junction and direct injection, may be used to achieve the same nanoprecipitation.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid.

In some embodiments, the lipid nanoparticle comprises 5 mol %, 10 mol %, 15 mol %, 20 mol %, or 25 mol % non-cationic lipid.

In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, an ionizable amino lipid of the disclosure comprises a compound of Formula (I):

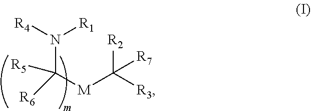

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

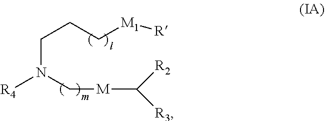

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

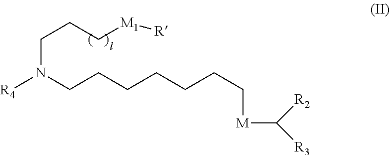

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

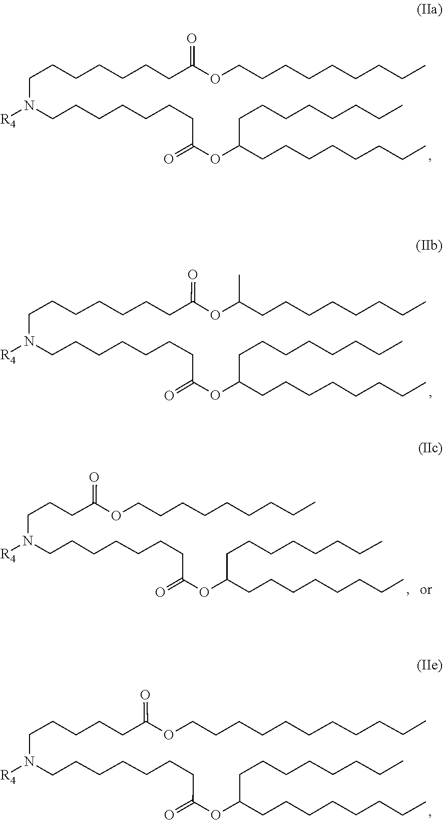

or a salt or isomer thereof, wherein R₄ is as described herein.

In some embodiments, a subset of compounds of Formula (I) includes those of Formula (IId):

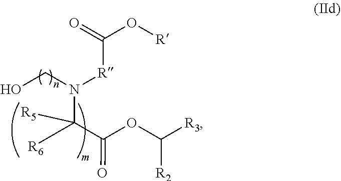

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

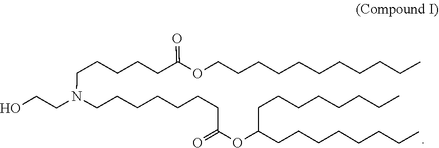 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

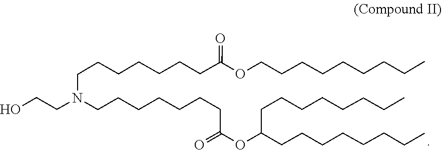 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

In some embodiments, the lipid nanoparticle comprises 5-15 mol % DSPC. For example, the lipid nanoparticle may comprise 5, 6, 7, 8, 9, 10, 11, 12, 13, 14, or 15 mol % DSPC.

In some embodiments, the lipid nanoparticle comprises 35-40 mol % cholesterol. For example, the lipid nanoparticle may comprise 35, 36, 37, 38, 39, or 40 mol % cholesterol.

In some embodiments, the lipid nanoparticle comprises 1-2 mol % DMG-PEG. For example, the lipid nanoparticle may comprise 1, 1.5, or 2 mol % DMG-PEG.

In some embodiments, the lipid nanoparticle comprises 50 mol % ionizable amino lipid, 10 mol % DSPC, 38.5 mol % cholesterol, and 1.5 mol % DMG-PEG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable amino lipid component to the RNA of from about 10:1 to about 100:1.

In some embodiments, a LNP of the disclosure comprises a wt/wt ratio of the ionizable amino lipid component to the RNA of about 20:1.

In some embodiments, a LNP of the disclosure comprises a wt/wt ratio of the ionizable amino lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The compositions, as provided herein, may include RNA or multiple RNAs encoding two or more antigens of the same or different species. In some embodiments, composition includes an RNA or multiple RNAs encoding two or more coronavirus antigens. In some embodiments, the RNA may encode 1, 2, 3, 4, 5, 6, 7, 8, 9, 10, 11, 12, or more coronavirus antigens. In some embodiments, the composition may comprise RNA or multiple RNAs encoding four antigens e.g., an OC43 antigen, an HKU1 antigen, and 229E antigen, and an NL63 antigen.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

Embodiments of the present disclosure provide compositions comprising combination vaccines (e.g., combination mRNA vaccines). A “combination vaccine” of the present disclosure refers to a vaccine comprising at least two polynucleotides, each comprising an open reading frame encoding at least one human coronavirus antigenic polypeptide (e.g., a human coronavirus S protein). In some embodiments, the combination vaccine comprises 2-10 mRNA polynucleotides, for example, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, 8 or 9-10 mRNA polynucleotides. In some embodiments, the combination vaccine comprises 2, 3, 4, 5, 6, 7, 8, 9, or 10 mRNA polynucleotides. In a particular embodiment, all the mRNAs encode viral surface proteins, e.g., glycoproteins, involved in receptor binding to facilitate viral entry into host cells (e.g., S proteins of human coronaviruses). In some embodiments, all of the mRNAs encode S proteins of human coronaviruses that comprise a double proline stabilizing mutation.

In some embodiments, the mRNAs encoding the human coronavirus antigens are present in the formulation in an equal amount (e.g., a 1:1 ratio), for example, a 1:1 ratio of mRNAs encoding distinct human coronavirus antigens (e.g., from two different human coronaviruses). In an exemplary vaccine comprising mRNAs encoding four different S protein antigens, mRNAs at a “1:1 ratio” would include the mRNAs in a ratio of 1:1:1:1 of the first, second, third and fourth mRNA.

In some embodiments, the first, second and third mRNA polynucleotides are present in the combination vaccine in a ratio of 1:1:1:1. In some embodiments, the combination vaccine comprises a ratio of mRNA polynucleotides encoding human coronavirus antigenic polypeptides of 4:1:1:1 from the first virus (e.g., OC43) to the second virus (e.g., HKU1) to the third virus (e.g., 229E) to the fourth virus (e.g., NL63). In some embodiments, the combination vaccine comprises a ratio of mRNA polynucleotides encoding human coronavirus antigenic polypeptides of 3:1:1:1 from the first virus (e.g., OC43) to the second virus (e.g., HKU1) to the third virus (e.g., 229E) to the fourth virus (e.g., NL63). In some embodiments, the combination vaccine comprises a ratio of mRNA polynucleotides encoding human coronavirus antigenic polypeptides of 2:1:1:1 from the first virus (e.g., OC43) to the second virus (e.g., HKU1) to the third virus (e.g., 229E) to the fourth virus (e.g., NL63).

In some embodiments, each of the mRNA polynucleotides in the combination vaccine is complementary with and does not interfere with each other mRNA polynucleotide in the combination vaccine. That is, an antigen produced from administration of the combination vaccine do not significantly interfere with the immune response to any other of the antigens produced in response to the vaccine in such a way that would diminish the ability of the antigens to provoke a protective immune response in a subject. In some embodiments, the combination vaccine is additive with respect to neutralizing antibodies relative to each individual antigen in a vaccine.

In each embodiment or aspect of the invention, it is understood that the featured vaccines include the mRNAs encapsulated within LNPs. While it is possible to encapsulate each unique mRNA in its own LNP, the mRNA vaccine technology enjoys the significant technological advantage of being able to encapsulate several mRNAs in a single LNP product.

### Pharmaceutical Formulations

Provided herein are compositions (e.g., pharmaceutical compositions), methods, kits and reagents for prevention or treatment of coronavirus in humans and other mammals, for example. The compositions provided herein can be used as therapeutic or prophylactic agents. They may be used in medicine to prevent and/or treat a coronavirus infection.

In some embodiments, the coronavirus vaccine containing RNA as described herein can be administered to a subject (e.g., a mammalian subject, such as a human subject), and the RNA polynucleotides are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing RNA polynucleotides having at least one chemical modification are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of a coronavirus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, a composition may be administered intramuscularly, intranasally or intradermally, similarly to the administration of inactivated vaccines known in the art.

The vaccine may be administered to seropositive or seronegative subjects. For example, a subject may be naïve and not have antibodies that react with a virus having an antigen, wherein the antigen is the viral antigen or fragment thereof encoded by the mRNA of the vaccine. Such a subject is said to be seronegative with respect to that vaccine. Alternatively, the subject may have preexisting antibodies to viral antigen encoded by the mRNA of the vaccine because they have previously had an infection with virus carrying the antigen or may have previously been administered a dose of a vaccine (e.g., an mRNA vaccine) that induces antibodies against the antigen. Such a subject is said to be seropositive with respect to that vaccine. In some instances the subject may have been previously exposed to a virus but not to a specific variant or strain or clade of the virus or a specific vaccine associated with that variant or strain or clade. Such a subject is considered to be seronegative with respect to the specific variant or strain or clade.

Thus, the present disclosure provides compositions (e.g., mRNA vaccines) that elicit potent neutralizing antibodies against a first antigen (e.g., a first human coronavirus antigen) and a second antigen (e.g., a second human coronavirus antigen) in a subject. Such a composition can be administered to seropositive or seronegative subjects in some embodiments. A seronegative subject may be naïve and not have antibodies that react with the specific virus which the subject is being immunized against. A seropositive subject may have preexisting antibodies to the specific virus because they have previously had an infection with that virus, variant or strain or clade or may have previously been administered a dose of a vaccine (e.g., an mRNA vaccine) that induces antibodies against that virus, variant or strain or clade.

In some embodiments, a subject may be naïve (e.g., seronegative) and not have antibodies that react with coronavirus antigenic polypeptides of the vaccine, or may have preexisting antibodies to coronavirus (e.g., NL63, OC43, 229E and/or HKU1) antigenic polypeptides of the vaccine because they have previously had an infection with the coronavirus or may have previously been administered a dose of a vaccine (e.g., an mRNA vaccine) that induces antibodies against the coronavirus (e.g., NL63, OC43, 229E and/or HKU1). In some embodiments, the subject may be seronegative for 1, 2, 3, or all 4 of the human coronavirus antigens encoded by the mRNAs in the vaccine composition. In some embodiments, the subject may be seropositive for 1, 2, 3, or all 4 of the human coronavirus antigens encoded by the mRNAs in the composition.

A composition may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants.

In some embodiments, an immunizing composition does not include an adjuvant (they are adjuvant free).

An RNA may be formulated or administered in combination with one or more pharmaceutically-acceptable excipients. In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Provided herein are immunizing compositions (e.g., RNA vaccines), methods, kits and reagents for prevention and/or treatment of coronavirus infection in humans and other mammals. Immunizing compositions can be used as therapeutic or prophylactic agents. In some embodiments, immunizing compositions are used to provide prophylactic protection from coronavirus infection. In some embodiments, immunizing compositions are used to treat a coronavirus infection. In some embodiments, embodiments, immunizing compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, an immunizing composition (e.g., RNA a vaccine) is administered to a subject (e.g., a mammalian subject, such as a human subject) in an effective amount to induce an antigen-specific immune response. The RNA encoding the coronavirus antigen is expressed and translated in vivo to produce the antigen, which then stimulates an immune response in the subject.

Prophylactic protection from a coronavirus can be achieved following administration of an immunizing composition (e.g., an RNA vaccine) of the present disclosure. Immunizing compositions can be administered once, twice, three times, four times or more but it is likely sufficient to administer the vaccine once (optionally followed by a single booster). It is possible, although less desirable, to administer immunizing compositions to an infected individual to achieve a therapeutic response. Dosing may need to be adjusted accordingly.

A method of eliciting an immune response in a subject against a coronavirus antigen (or multiple antigens) is provided in aspects of the present disclosure. In some embodiments, a method involves administering to the subject an immunizing composition comprising a RNA (e.g., mRNA) having an open reading frame encoding a coronavirus antigen, thereby inducing in the subject an immune response specific to the coronavirus antigen, wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the coronavirus or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the coronavirus or an unvaccinated subject.

A method of eliciting an immune response in a subject against a coronavirus is provided in other aspects of the disclosure. The method involves administering to the subject an immunizing composition (e.g., an RNA vaccine) comprising a RNA polynucleotide comprising an open reading frame encoding a coronavirus antigen, thereby inducing in the subject an immune response specific to the coronavirus, wherein the immune response in the subject is equivalent to an immune response in a subject vaccinated with a traditional vaccine against the coronavirus at 2 times to 100 times the dosage level relative to the immunizing composition.

In some embodiments, the immune response in the subject is equivalent to an immune response in a subject vaccinated with a traditional vaccine at twice the dosage level relative to an immunizing composition of the present disclosure. In some embodiments, the immune response in the subject is equivalent to an immune response in a subject vaccinated with a traditional vaccine at three times the dosage level relative to an immunizing composition of the present disclosure. In some embodiments, the immune response in the subject is equivalent to an immune response in a subject vaccinated with a traditional vaccine at 4 times, 5 times, 10 times, 50 times, or 100 times the dosage level relative to an immunizing composition of the present disclosure. In some embodiments, the immune response in the subject is equivalent to an immune response in a subject vaccinated with a traditional vaccine at 10 times to 1000 times the dosage level relative to an immunizing composition of the present disclosure. In some embodiments, the immune response in the subject is equivalent to an immune response in a subject vaccinated with a traditional vaccine at 100 times to 1000 times the dosage level relative to an immunizing composition of the present disclosure.

In other embodiments, the immune response is assessed by determining [protein] antibody titer in the subject. In other embodiments, the ability of serum or antibody from an immunized subject is tested for its ability to neutralize viral uptake or reduce coronavirus transformation of human B lymphocytes. In other embodiments, the ability to promote a robust T cell response(s) is measured using art recognized techniques.

Other aspects the disclosure provide methods of eliciting an immune response in a subject against a coronavirus by administering to the subject an immunizing composition (e.g., an RNA vaccine) comprising an RNA having an open reading frame encoding a coronavirus antigen, thereby inducing in the subject an immune response specific to the coronavirus antigen, wherein the immune response in the subject is induced 2 days to 10 weeks earlier relative to an immune response induced in a subject vaccinated with a prophylactically effective dose of a traditional vaccine against the coronavirus. In some embodiments, the immune response in the subject is induced in a subject vaccinated with a prophylactically effective dose of a traditional vaccine at 2 times to 100 times the dosage level relative to an immunizing composition of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a coronavirus by administering to the subject an RNA having an open reading frame encoding a first antigen, wherein the RNA does not include a stabilization element, and wherein an adjuvant is not co-formulated or co-administered with the vaccine.

An immunizing composition (e.g., an RNA vaccine) may be administered by any route that results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the RNA, as provided herein, may be as low as 20 μg, administered for example as a single dose or as two 10 μg doses. In some embodiments, the effective amount is a total dose of 20 μg-300 μg or 25 μg-300 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount is a total dose of 20 μg. In some embodiments, the effective amount is a total dose of 25 μg. In some embodiments, the effective amount is a total dose of 75 μg. In some embodiments, the effective amount is a total dose of 150 μg. In some embodiments, the effective amount is a total dose of 300 μg.

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the immunizing compositions (e.g., RNA vaccines), wherein the RNA is formulated in an effective amount to produce an antigen specific immune response in a subject (e.g., production of antibodies specific to a coronavirus antigen). “An effective amount” is a dose of the RNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) coronavirus protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function, and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-coronavirus antigen antibody titer produced in a subject administered an immunizing composition as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, SAR-CoV-2 virus or SAR-CoV-2 viral antigen, e.g., SAR-CoV-2 spike or S protein, of domain thereof. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection.

The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold titer. Regarding SARS-CoV-2 neutralizing antibodies, the “seroprotective” threshold titer remains unknown; but a seropositivity threshold of 1:10 can be considered a seroprotection threshold in certain embodiments.

PRNT end-point titers are expressed as the reciprocal of the last serum dilution showing the desired percent reduction in plaque counts. The PRNT titer can be calculated based on a 50% or greater reduction in plaque counts (PRNT50). A PRNT50 titer is preferred over titers using higher cut-offs (e.g., PRNT90) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by an immunizing composition (e.g., RNA vaccine).

In some embodiments, an anti-coronavirus antigen antibody titer produced in a subject is increased by at least 1 log relative to a control. For example, anti-coronavirus antigen antibody titer produced in a subject may be increased by at least 1.5, at least 2, at least 2.5, or at least 3 log relative to a control. In some embodiments, the anti-coronavirus antigen antibody titer produced in the subject is increased by 1, 1.5, 2, 2.5 or 3 log relative to a control. In some embodiments, the anti-coronavirus antigen antibody titer produced in the subject is increased by 1-3 log relative to a control. For example, the anti-coronavirus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-coronavirus antigen antibody titer produced in a subject is increased at least 2 times relative to a control. For example, the anti-coronavirus antigen n antibody titer produced in a subject may be increased at least 3 times, at least 4 times, at least 5 times, at least 6 times, at least 7 times, at least 8 times, at least 9 times, or at least 10 times relative to a control. In some embodiments, the anti-coronavirus antigen antibody titer produced in the subject is increased 2, 3, 4, 5, 6, 7, 8, 9, or 10 times relative to a control. In some embodiments, the anti-coronavirus antigen antibody titer produced in a subject is increased 2-10 times relative to a control. For example, the anti-coronavirus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to coronavirus. A geometric mean titer (GMT) is the average antibody titer for a group of subjects calculated by multiplying all values and taking the nth root of the number, where n is the number of subjects with available data.

A control, in some embodiments, is an anti-coronavirus antigen antibody titer produced in a subject who has not been administered an immunizing composition (e.g., RNA vaccine). In some embodiments, a control is an anti-coronavirus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of an immunizing composition (e.g., RNA vaccine) to be effective is measured in a murine model. For example, an immunizing composition may be administered to a murine model and the murine model assayed for induction of neutralizing antibody titers. Viral challenge studies may also be used to assess the efficacy of a vaccine of the present disclosure. For example, an immunizing composition may be administered to a murine model, the murine model challenged with virus, and the murine model assayed for survival and/or immune response (e.g., neutralizing antibody response, T cell response (e.g., cytokine response)).

In some embodiments, an effective amount of an immunizing composition (e.g., RNA vaccine) is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent coronavirus infection or a related condition, while following the standard of care guideline for treating or preventing coronavirus infection or a related condition.

In some embodiments, the anti-coronavirus antigen antibody titer produced in a subject administered an effective amount of an immunizing composition is equivalent to an anti-coronavirus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$\begin{matrix}Efficacy = ( A R U - A RV ) / ARU \times 100 ; \\ and \\ Efficacy = ( 1 - RR ) \times 100.\end{matrix}$$

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In some embodiments, efficacy of the immunizing composition (e.g., RNA vaccine) is at least 60% relative to unvaccinated control subjects. For example, efficacy of the immunizing composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of an immunizing composition of the present disclosure is sufficient to provide sterilizing immunity in the subject for at least 1 year. For example, the effective amount of an immunizing composition of the present disclosure is sufficient to provide sterilizing immunity in the subject for at least 2 years, at least 3 years, at least 4 years, or at least 5 years. In some embodiments, the effective amount of an immunizing composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of an immunizing composition of the present disclosure is sufficient to produce detectable levels of coronavirus antigen as measured in serum of the subject at 1-72 hours post administration.

Titer. An antibody titer is a measurement of the amount of antibodies within a subject, for example, antibodies that are specific to a particular antigen (e.g., an anti-coronavirus antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of an immunizing composition of the present disclosure is sufficient to produce a 1,000-10,000 neutralizing antibody titer produced by neutralizing antibody against the coronavirus antigen as measured in serum of the subject at 1-72 hours post administration. In some embodiments, the effective amount is sufficient to produce a 1,000-5,000 neutralizing antibody titer produced by neutralizing antibody against the coronavirus antigen as measured in serum of the subject at 1-72 hours post administration. In some embodiments, the effective amount is sufficient to produce a 5,000-10,000 neutralizing antibody titer produced by neutralizing antibody against the coronavirus antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-coronavirus antigen antibody titer produced in the subject is increased by at least 1 log relative to a control. For example, an anti-coronavirus antigen antibody titer produced in the subject may be increased by at least 2, 3, 4, 5, 6, 7, 8, 9 or 10 log relative to a control.

In some embodiments, an anti-coronavirus antigen antibody titer produced in the subject is increased at least 2 times relative to a control. For example, an anti-coronavirus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### EXAMPLES

### Example 1: In Vivo Expression of Antigens Encoded by mRNA Vaccines

BALB/c mice, 6-8 weeks of age, are administered either 2 μg or 10 μg of a COVID-19 construct or Tris buffer (as a control) intramuscularly in each hind leg. The constructs comprise any of the mRNA encoding antigens disclosed herein in cationic (amino) lipid nanoparticles, 10.7 mM sodium acetate, 8.7% sucrose, 20 mM Tris (pH 7.5). One day later, spleens and lymph nodes are collected to detect protein expression using flow cytometry.

### Example 2: Multiantigen Dose Response, Immune Interference, and Immunogenicity Analysis Using mRNAs Encoding Four Human Coronavirus Antigens

In this example, multiple human coronavirus antigens administered as mRNA vaccines are evaluated for immunogenicity, dose response, and immunological interference between the antigens. Further, the data is examined for any dampening of immune response towards one antigen over another different antigen when all antigens are co-administered simultaneously as mRNA vaccines formulated in lipid nanoparticles (LNPs). For this study, the antigens are formulated separately into different LNPs and mixed before administration. The experiment is carried out in both a high dose version (2-16 μg mRNA/animal) and a low dose version (0.4-3.6 μg mRNA/animal). Different ratios of antigens (e.g., 1:1:1:1 and 2:1:1:1) are tested.

The immunization regimen is a prime on day 1 and boost on day 21 with the same amount of specified vaccine. Antibody responses are evaluated by ELISA (IgG antibody binding titers) on day 21 (3 weeks post dose 1) and day 36 (2 weeks post dose 2). Briefly, the ELISA plates are coated with individual recombinant protein antigens at various concentrations (5 μg/ml, 2 μg/ml, or 1 μg/ml.) Antibody titers are determined using a four-parameter logistic curve fit in GraphPad Prism (GraphPad Software, Inc.) and defined as the reciprocal dilution at approximately OD450 nm=1.5 (normalized to a mouse standard on each plate).

### Example 3: In Vitro Expression

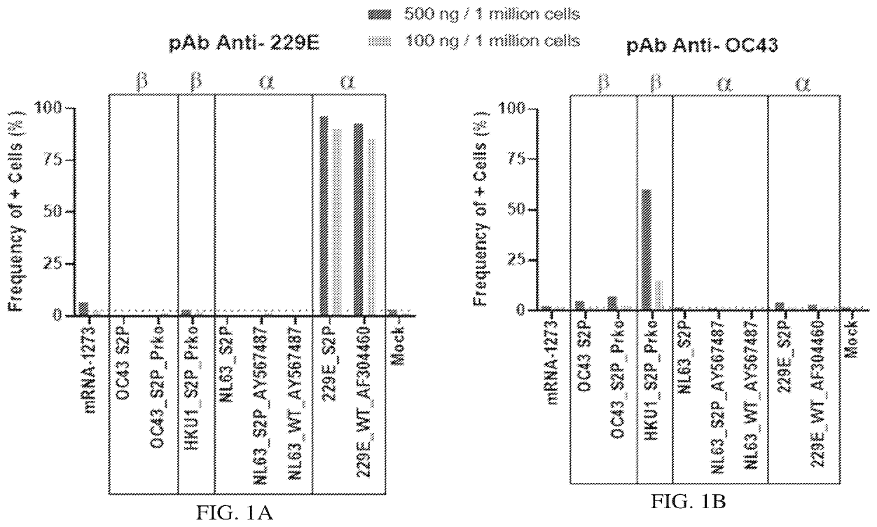

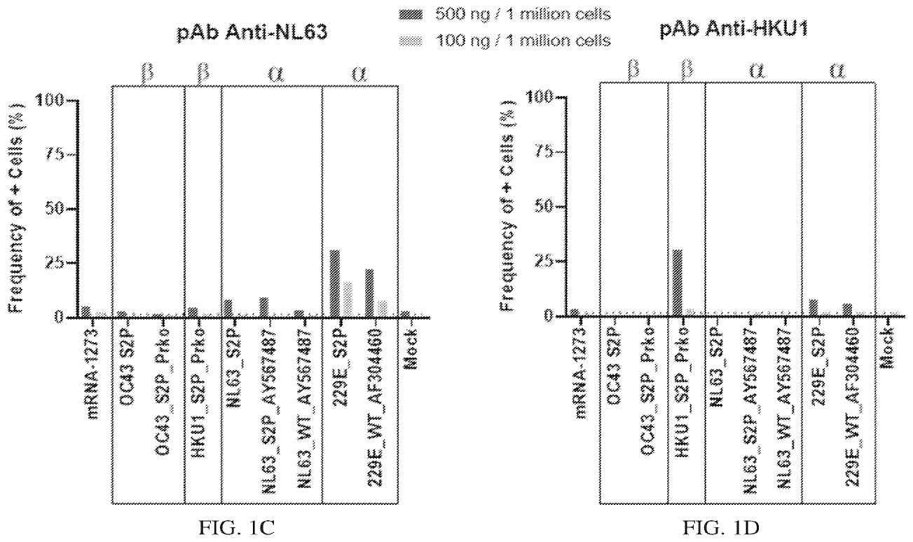

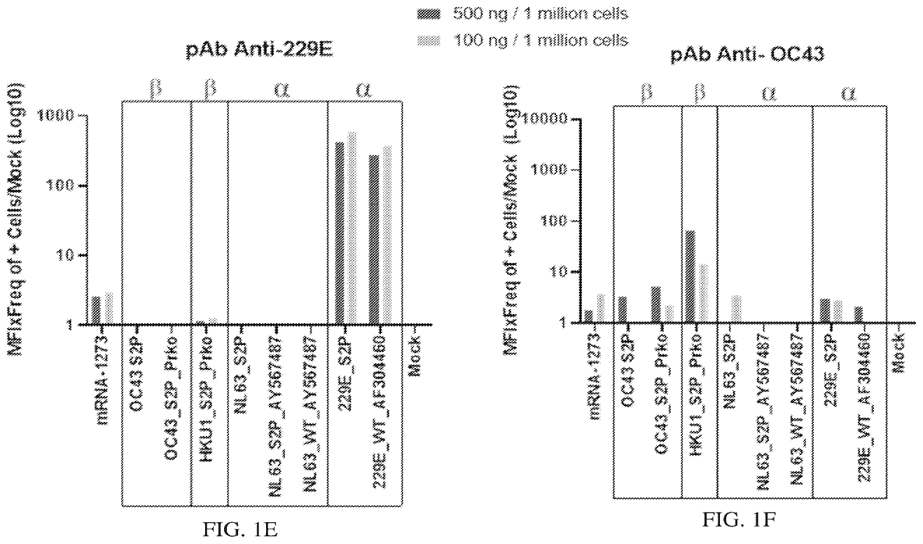

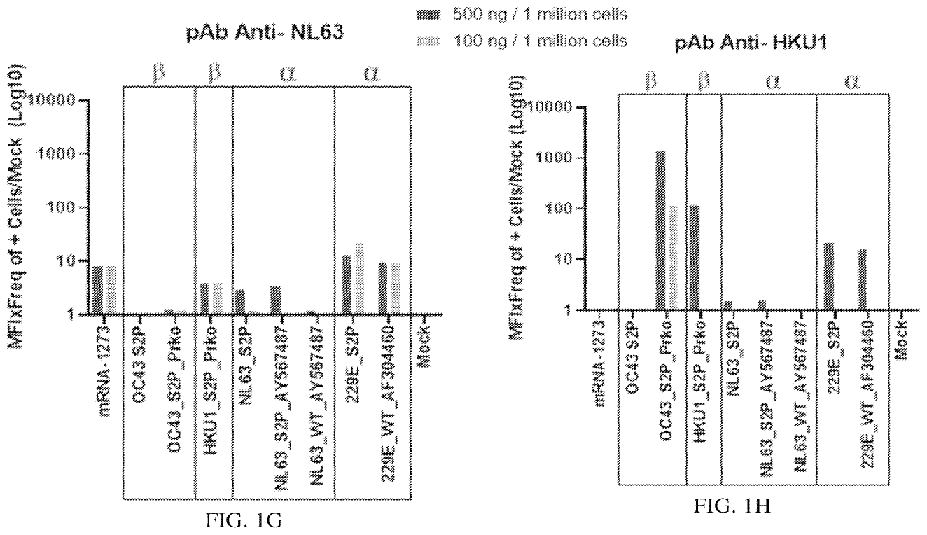

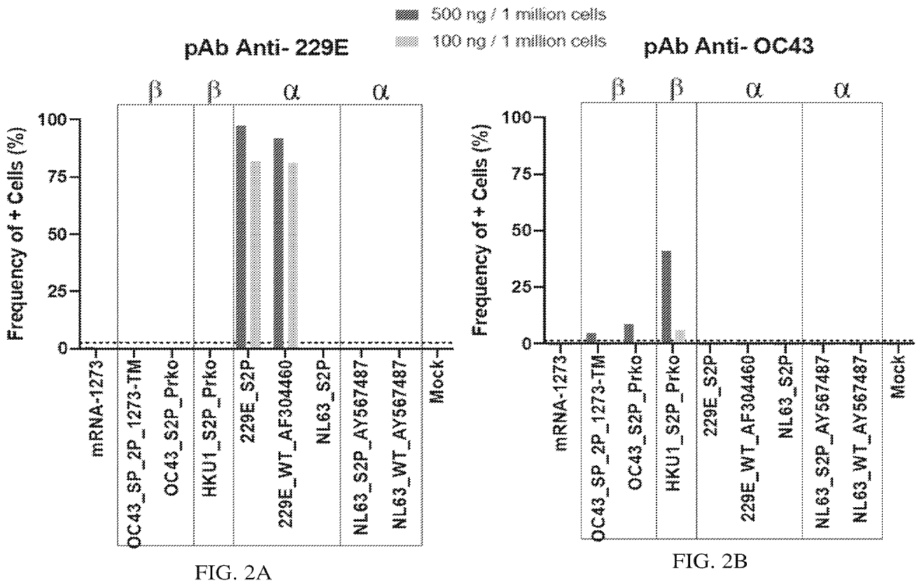

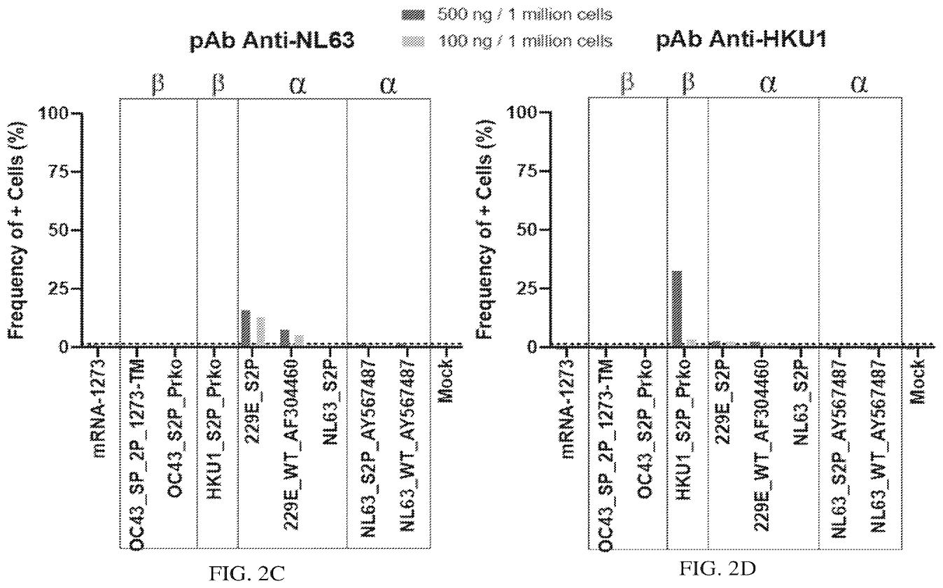

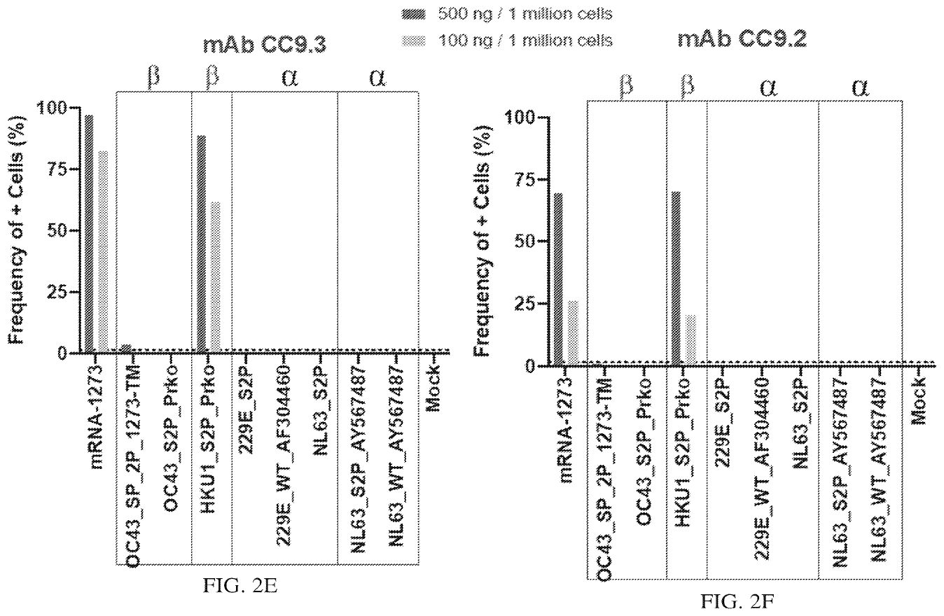

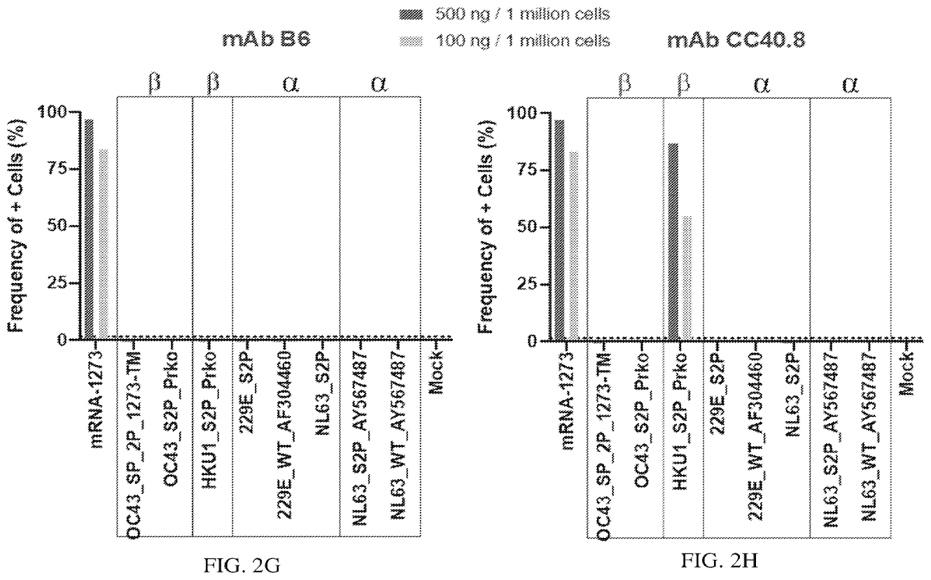

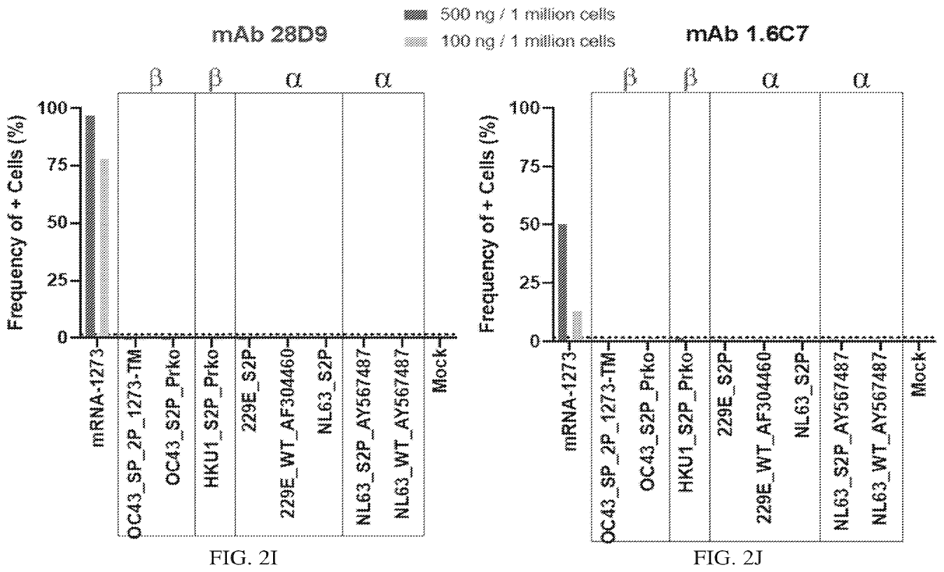

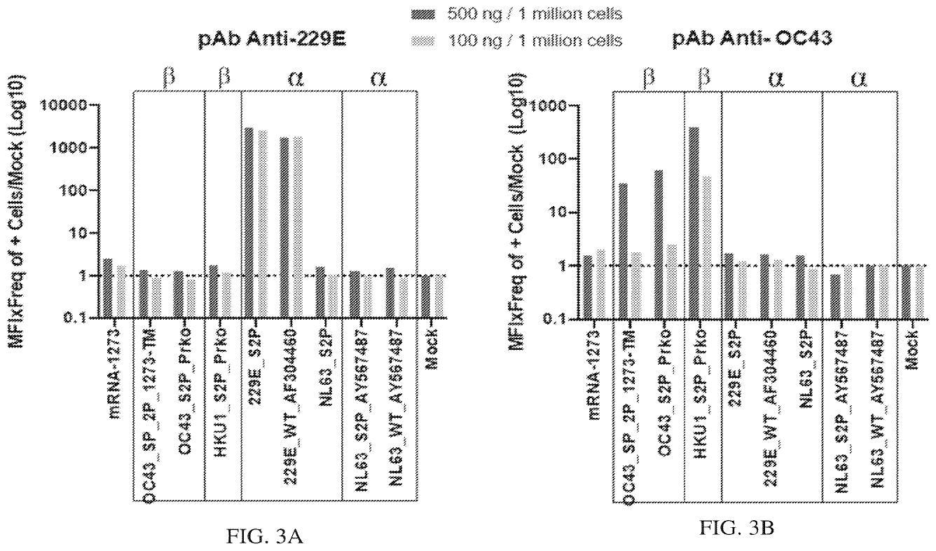

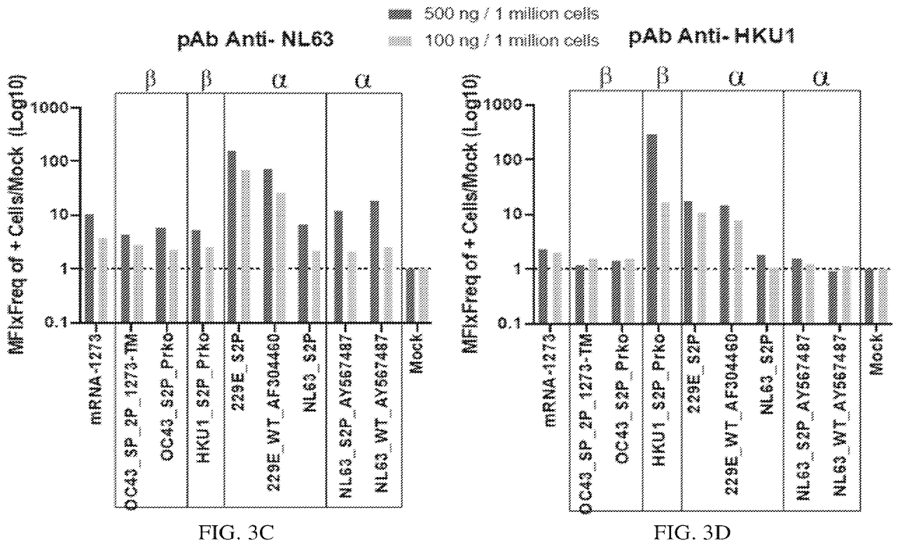

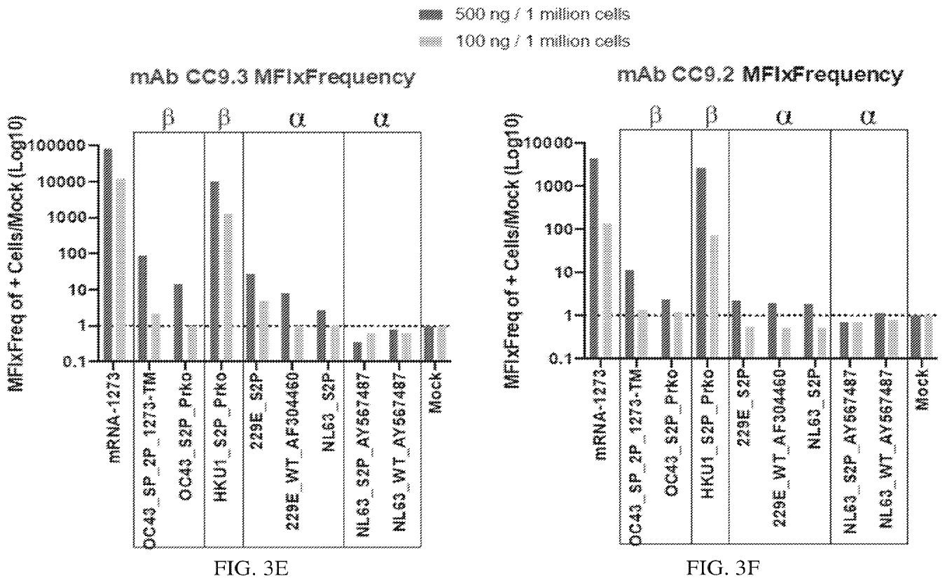

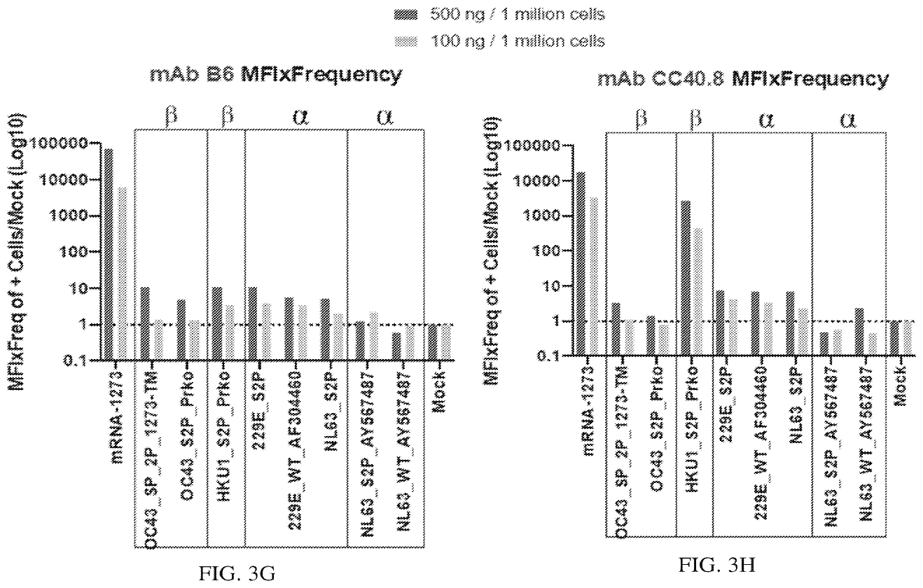

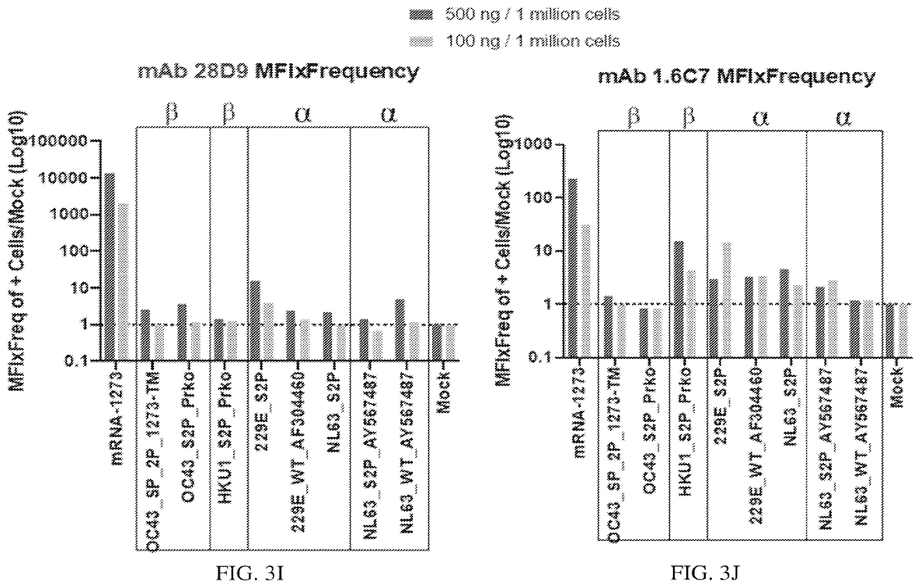

This Example demonstrates that the compositions described herein resulted in in vitro expression on cell surfaces. Briefly, EXPI293 cells were plated in a 6-well plate with 5 mL of culture, and 1×10⁶ cells/mL were added to each well. Transfection was performed using with the TranslT®-mRNA Transfection Kit using 2 μL per 1 μg of mRNA. The following mRNA formulations were tested: mRNA-1273 (mRNA encoding a SARS-CoV-2 spike protein comprising a double proline stabilizing mutation), OC43 S2P, OC43_S2P_Prko, HKU1_S2P_Prko, NL63_S2P, NL63_S2P_AY567487, NL63_WT_AY567487, 229E_S2P, 229E_WT_AF304460. The amount of mRNA added per well was either 500 ng per 1 million cells (2.5 μg/well) or 100 ng per 1 million cells (0.5 μg/well). The plates were shaken in an incubator for 48 hours at 120 rpm. After 48 hours, the cells were harvested, stained, and fixed with BD Cytofix. The antibodies used for the analysis are described in Table 2. The readout was performed with the LSR Fortessa. The results are shown in FIGS. 1A-1H, 2A-2J, 3A-3J, and 4A-4D), and demonstrate that, in cells transfected with virus-specific mRNA, the corresponding antibody was able to detect the presence of the corresponding protein on the surface of the cells.

TABLE 2
Antibodies used for in vitro screening

| Polyclonal Antibodies | Dilution | Source |
|---|---|---|
| Rabbit IgG Anti HCoV-229E Spike | 1/500 | PAB21477-100 |
| (S1) Polyclonal Antibody | | (Native Antigen) |
| Rabbit IgG Anti HCoV-OC43 Spike | 1/500 | PAB21478-100 |
| (S1) Polyclonal Antibody | | (Native Antigen) |
| Rabbit IgG Anti HCoV-NL63 Spike | 1/500 | PAB21479-100 |
| (S1) Polyclonal Antibody | | (Native Antigen) |
| Rabbit IgG Anti HCoV-HKU1 Spike | 1/500 | PAB21480-100 |
| (S1) Polyclonal Antibody | | (Native Antigen) |
| Monoclonal Antibodies | Dilution | Source |
| CC9.3 (Human) | 1/1000 | Nature Structural |
| | | & Molecular |
| | | Biology (2021) |
| CC9.2 (Human) | 1/1000 | Nature |
| | | Communications |
| | | (2021) |
| B6 (Human) | 1/1000 | Nature |
| | | Communications |
| | | (2021) |
| CC40.8 (Human) | 1/1000 | Nature |
| | | Communications |
| | | (2021) |
| 28D9 (Human) | 1/1000 | Nature |
| | | Communications |
| | | (2021) |
| 1.6C7 (Human) | 1/1000 | Nature |
| | | Communications |
| | | (2021) |
| Secondary Antibody | Dilution | CAT # |
| Goat anti-Rabbit IgG AF647 | 1/1000 | A32733 |
| Goat anti-Human IgG AF647 | 1/1000 | OB201631 |

### Example 4: Sera Anti-Spike Protein Antigen Binding

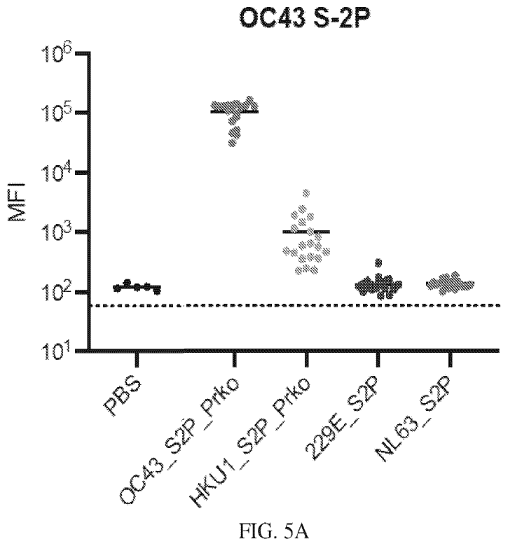

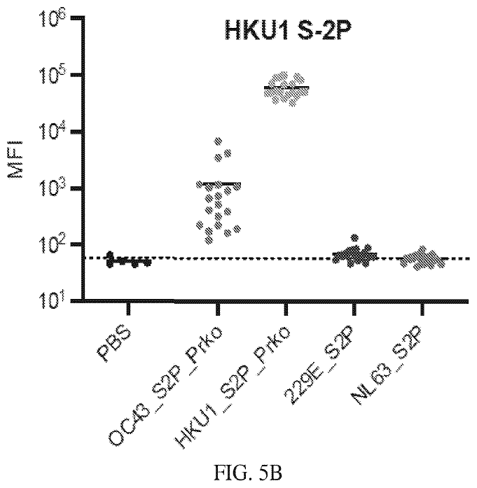

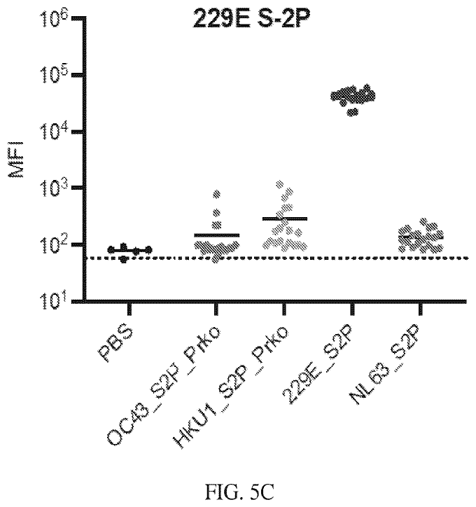

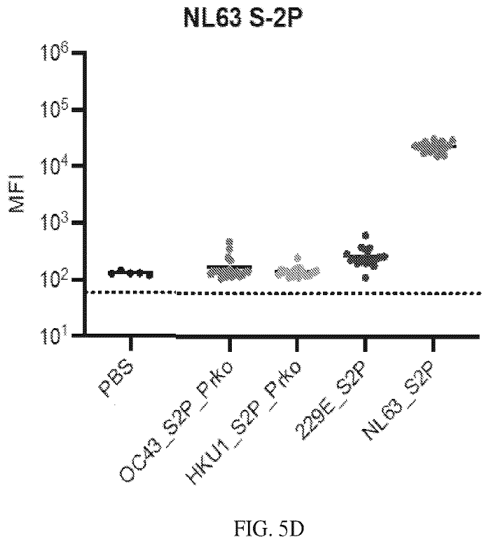

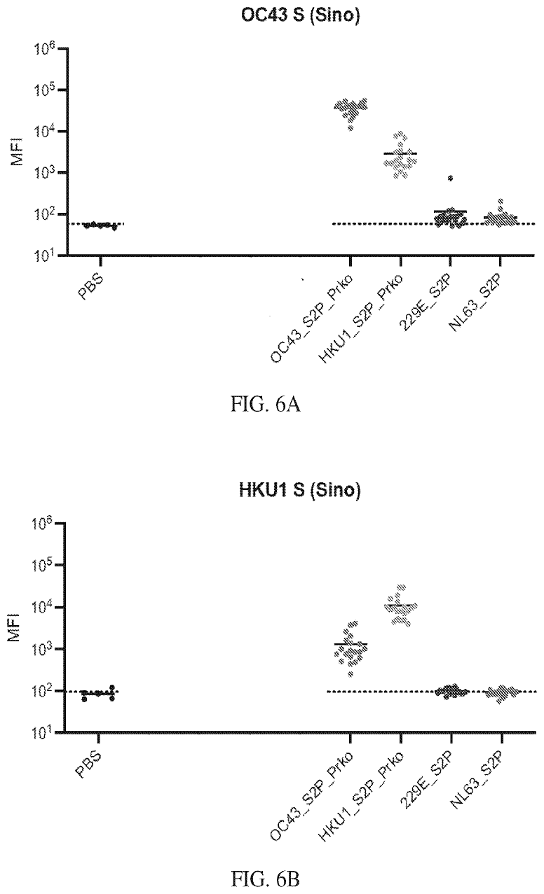

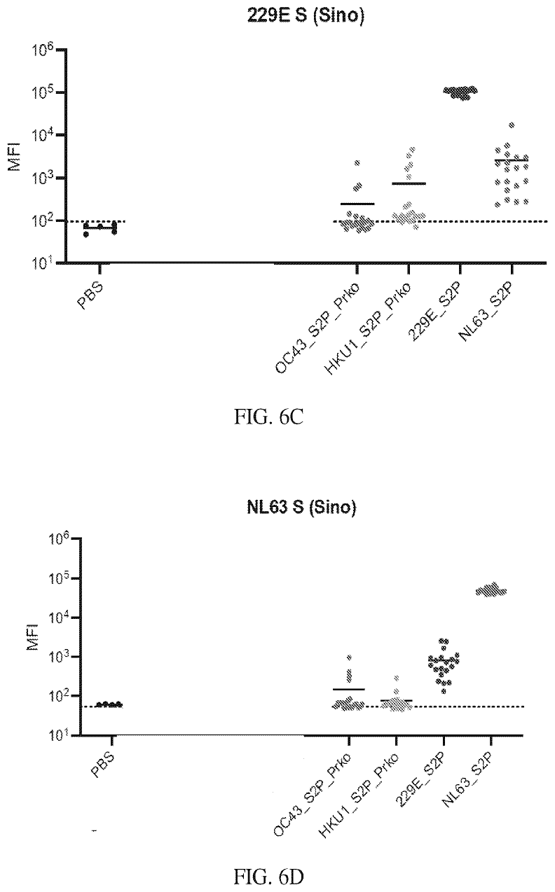

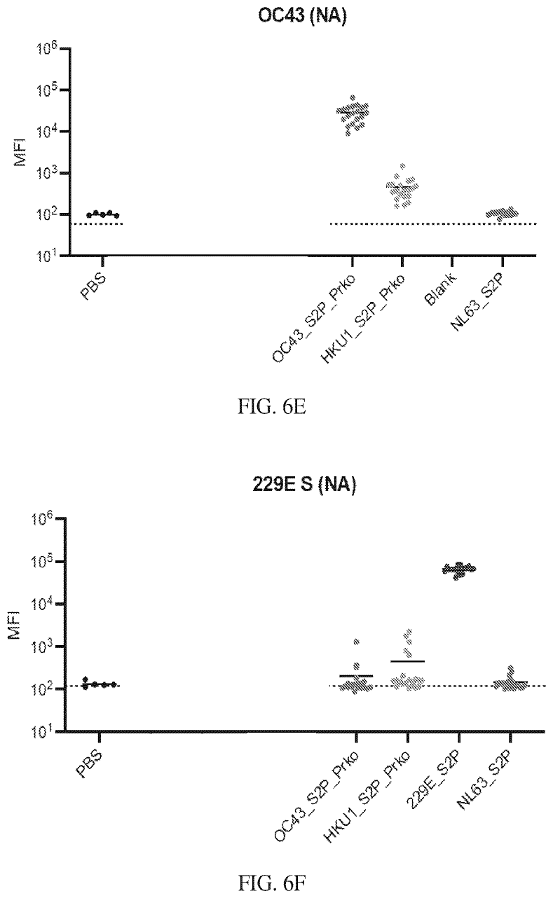

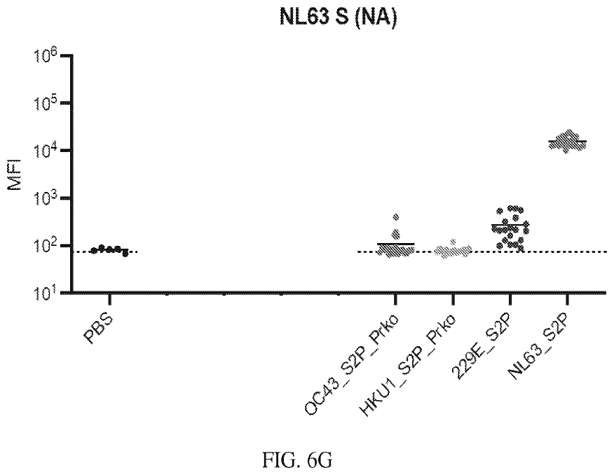

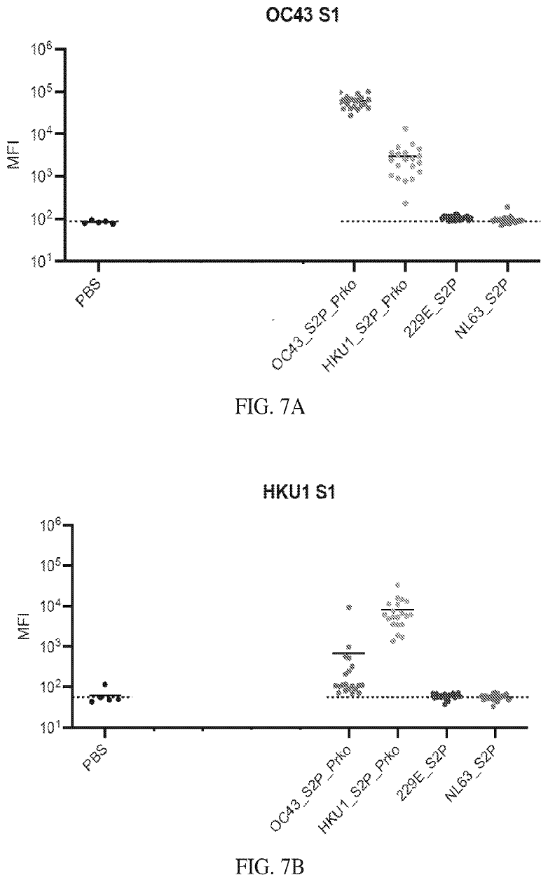

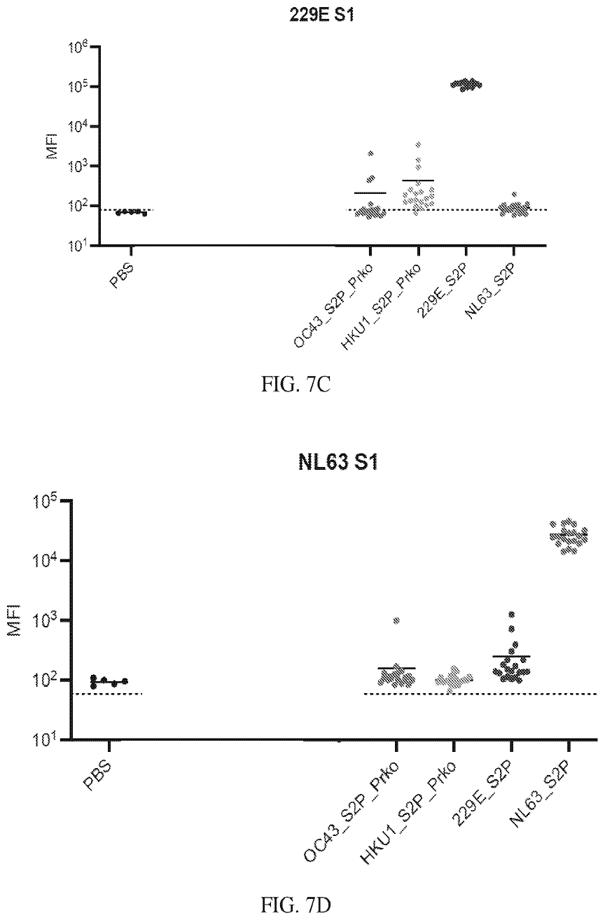

This study looked at whether “hyperimmune” sera against endemic human coronaviruses could be produced in vivo. On days 1 and 22, BALB/c mice were vaccinated intra-muscularly (IM) with 10 μg of mRNA encoding OC4_S2P_Prko, NL63_S2P, 229E_SP, or HKU_S2P_Prko to produce sera against endemic human coronaviruses (hCoV). The number of mice vaccinated per group was 20. On day 36, sera were collected for neutralization and Luminex analysis. Binding was examined for S-2P antigens (FIGS. 5A-5D), S antigens (FIGS. 6A-6G), and S1 antigens (FIGS. 7A-7D), and each antigen tested was found to be present in the mouse samples (as demonstrated by mean fluorescence intensity). Very limited cross-reactivity was observed between alpha- and beta-coronaviruses.

Pooled sera were used to examine neutralization in pseudovirus assays. OC43, 229E, and NL63 pseudovirus neutralization assays were performed. Briefly, VSVΔG-based pseudoviruses (PsV) were generated for each virus and then cells were infected with each pseudovirus separately and then neutralization was measured. Neutralization was observed for each pseudovirus with each corresponding mRNA formulation. The results are shown in Table 3 below. In particular, some neutralization was observed with the VSV PsV expressing OC43-d19 spike protein. In studies with the VSV pseudovirus expressing the 229E-d19 spike protein, neutralizing titers were observed for S2P. Further, studies were conducted with the VSV PsV expressing NL63-d19 spike protein, that was derived from the historical (BEI) sequence, in both Huh-7 (Human hepatocyte carcinoma) and LLC-MK2 (Macaque kidney epithelial) cells. High neutralizing titers were observed in both cell lines. (Table 3) and very limited cross-reactivity was observed between alpha- and beta-coronaviruses.

TABLE 3
Pseudovirus Neutralization Titers

| | |
|---|---|
| Compound | NT50 |

OC43

| | |
|---|---|
| PBS | 56 |
| OC43-S2P | 488 |
| NL63-S2P | 124 |
| 229E-S2P | 179 |
| HKU1-S2P | 142 |

229E

| | |
|---|---|
| PBS | 42 |
| OC43-S2P | 39 |
| NL63-S2P | 429 |
| 229E-S2P | 6706 |
| HKU1-S2P | 39 |

NL63_Huh7 (human hepatocyte carcinoma cells)

| | |
|---|---|
| PBS | 39 |
| OC43-S2P | 78 |
| NL63-S2P | 176622 |
| 229E-S2P | 262 |
| HKU1-S2P | 532 |

NL63_LLC (Macaque kidney epithelial cells)

| | |
|---|---|
| PBS | 39 |
| OC43-S2P | 77 |
| NL63-S2P | 101166 |
| 229E-S2P | 319 |
| HKU1-S2P | 74 |

### Example 5: Endemic hCoV Virus Neutralization Studies

These studies examined the neutralization capacity of antibodies resulting from immunization with OC43_S2P_Prko, NL63_S2P, 229E_S2P, and HUK1_S2P antigens (Table 4). On days 1 and 22, BALB/c mice were vaccinated intra-muscularly (IM) with 10 μg of mRNA encoding OC43_S2P_Prko, NL63_S2P, 229E_S2P, or HKU1_S2P to produce sera against endemic human coronaviruses (hCoV). The immunization regimen is a prime on day 1 and boost on day 21 with the same amount of specified vaccine. Antibody responses were evaluated by ELISA (IgG antibody binding titers) on day 21 (3 weeks post dose 1) and day 36 (2 weeks post dose 2). OC43 authentic virus was generated in RD cells (Rhabdomyosarcoma; muscle), 229E authentic virus was generated in Huh7 cells (Human hepatocyte carcinoma), and NL63 authentic virus was generated in LLC-MK2 cells (Macaque kidney epithelial). The night prior to performing the foci reduction neutralization test (FRNT), cells were seeded in 96 well plates at a density of 3e4 cells per well. Sera collected from immunized mice was serially diluted in DMEM+2% FBS starting at a 1:10 dilution and subsequently diluted 4-fold. Pooled sera were used to examine neutralization in authentic virus assays. Authentic viruses (OC43, 229E, or NL63) were diluted to a pre-determined concentration. Pooled sera and authentic virus were mixed at 1 part sera: 1 part virus, with a final starting dilution of 1:20. Pooled sera and virus mixtures were incubated for 1 hour at 370 Celsius. 50 μL of sera and virus mixtures were then added to cells in triplicate and incubated for 1 hour at 37° Celsius. Cells exposed to sera and virus mixtures were overlayed with 150 μL of methyl-cellulose. Cells were incubated at 33° Celsius for 24-48 hours. Cells were then fixed with 4% paraformaldehyde and immunolabeled with a nucleocapsid primary antibody. Cells were then immunolabeled with a horseradish peroxidase (HRP) conjugated secondary antibody. 50 μL of TrueBlue substrate was then added to the cells. 96 well plates containing immunolabeled cells with TrueBlue substrate were then scanned using CTL.

Neutralization was observed for authentic virus with each corresponding mRNA formulation. The results are shown in Table 5 below. Modest neutralization of the OC43 authentic virus was observed after immunization with OC43_S2P-Prko antigen. Neutralization titers for the 229E authentic virus were observed following immunization with 229E_S2P antigen. High neutralizing titers for the NL63 authentic virus were observed after immunization with NL63_S2P antigen. Modest neutralizing titers of NL63 authentic virus were observed following immunization with OC43_S2P_Prko, 229E_S2P, and HKU1_S2P antigens (Table 5).

TABLE 4
Experimental Groups 1273 Designs

| | | Dose | |
|---|---|---|---|
| Group | Material | (ug/ms) | #Mice |
| 1 | PBS | x | 5 |
| 2 | OC43_S2P_Prko | 10 | 20 |
| 3 | NL63_S2P | 10 | 20 |
| 4 | 229E_S2P | 10 | 20 |
| 5 | HKU1_S2P_Prko | 10 | 20 |
| | | Total: | 85 |

TABLE 5
Authentic Virus Neutralization Titers

| | |
|---|---|
| Compound | NT50 |

OC43 Authentic Virus_RD (Rhabdomyosarcoma; muscle cells)

| | |
|---|---|
| PBS | 19 |
| OC43_S2P_Prko | 162 |
| NL63_S2P | 19 |
| 229E_S2P | 19 |
| HKU1S2P | 19 |

229E Authentic Virus_Huh7 (Human hepatocyte carcinoma cells)

| | |
|---|---|
| PBS | 22 |
| OC43_S2P_Prko | 19 |
| NL63_S2P | 19 |
| 229E_S2P | 100 |
| HKU1_S2P | 19 |

NL63 Authentic Virus_LLC-MK2 (Macaque kidney epithelial cells)

| | |
|---|---|
| PBS | 43 |
| OC43_S2P_Prko | 152 |
| NL63_S2P | 19,052 |
| 229E_S2P | 268 |
| HKU1_S2P | 262 |

### Example 6: Historical Pseudovirus Neutralization Studies

These studies examined the neutralization capacity of antibodies resulting from immunization using historical and contemporary coronavirus immunogens. The neutralization studies included BetaCoV OC43 historical pseudovirus OC43_S2P_Prko_AY585228, OC43_WT_Prko_AY585228, OC43_S2P_Prko, OC43_SP_2P_1273-TM, and HKU_S2P_Prko. The AlphaCoV 229E historical pseudovirus included 229E_S2P_AF304460, 229E_WT_AF304460, and 229_S2P. The AlphaCoV NL63 historical pseudovirus included NL63_S2P_AY567487, NL63_WT_AY567, and NL63_S2P.

On days 1 and 22, BALB/c mice were vaccinated intra-muscularly (IM) with 10 μg of mRNA encoding one of the following coronavirus proteins: OC43_S2P_Prko_AY585228, OC43_WT_Prko_AY585228, OC43_S2P_Prko, OC43_SP_2P_1273-TM, HKU_S2P_Prko, 229E_S2P_AF304460, 229E_WT_AF304460, 229_S2P, NL63_S2P_AY567487, NL63_WT_AY567, or NL63_S2P. The immunization regimen was a prime on day 1 and boost on day 21 with the same amount of specified vaccine (Table 6). Antibody responses were evaluated by ELISA (IgG antibody binding titers) on day 21 (3 weeks post dose 1) and day 36 (2 weeks post dose 2).

BetaCoV OC43 historical pseudovirus was generated in RD cells (Rhabdomyosarcoma; muscle), AlphaCoV 229E historical pseudovirus was generated in Huh7 cells (Human hepatocyte carcinoma), and AlphaCoV NL63 historical pseudovirus was generated in LLC-MK2 cells (Macaque kidney epithelial). The night prior to performing the foci reduction neutralization test (FRNT), cells were seeded in 96 well plates at a density of 3e4 cells per well. Sera collected from vaccinated mice was serially diluted in DMEM+2% FBS starting at a 1:10 dilution and subsequently diluted 4-fold. Pooled sera were used to examine neutralization in authentic virus assays. Historical pseudoviruses (OC43, 229E, or NL63) were diluted to a pre-determined concentration. Pooled sera and authentic virus were mixed at 1 part sera: 1 part virus, with a final starting dilution of 1:20. Pooled sera and virus mixtures were incubated for 1 hour at 37° Celsius. 50 μL of sera and virus mixtures were then added to cells in triplicate and incubated for 1 hour at 37° Celsius. Cells exposed to sera and virus mixtures were overlayed with 150 μL of methyl-cellulose. Cells were incubated at 33° Celsius for 24-48 hours. Cells were then fixed with 4% paraformaldehyde and immunolabeled with a nucleocapsid primary antibody. Cells were then immunolabeled with a horseradish peroxidase (HRP) conjugated secondary antibody. 50 μL of TrueBlue substrate was then added to the cells. 96 well plates containing immunolabeled cells with TrueBlue substrate were then scanned using CTL.

Neutralization was observed for historical human pseudovirus with each corresponding mRNA formulation. OC43 immunogen formulations were examined using BetaCoV historical pseudovirus. 229E and NL63 immunogen formulations were examined using AlphaCoV historical 229E and NL63 pseudoviruses, respectively. The results are shown in Table 7 below. Neutralization was observed with the BetaCoV OC43 historical pseudovirus using OC43_S2P_AY585228, and OC43_S2P_Prko, OC43_WT_Prko_AY585228 antigens. Neutralization of the AlphaCoV 229E historical pseudovirus was observed using 229E_S2P_AF304460, 229E_WT_AF304460, 229E_S2P antigens. High neutralizing titers for AlphaCoV NL63 historical pseudovirus were observed for the NL63_S2P_AY567487 NL63_WT_AY567487, and NL63_S2P antigens (Table 7).

TABLE 6
mRNA immunogen 1273 Designs

| | | Dose | |
|---|---|---|---|
| Group | Material | (ug/ms) | #Mice |
| 1 | PBS | x | 8 |
| 2 | OC43_WT_Prko_AY585228 | 0.5 | 8 |
| 3 | OC43_S2P_Prko_AY585228 | 0.5 | 8 |
| 4 | OC43_S2P_Prko | 0.5 | 8 |
| 5 | 229E_WT_AF304460 | 0.5 | 8 |
| 6 | 229E_S2P_AF304460 | 0.5 | 8 |
| 7 | 229E_S2P | 0.5 | 8 |
| 8 | NL63_WT_AY567487 | 0.5 | 8 |
| 9 | NL63_S2P | 0.5 | 8 |
| 10 | NL63_S2P_AY567487 | 0.5 | 8 |
| | | Total: | 80 |

TABLE 7
Historical Pseudovirus Neutralization Titer

| | |
|---|---|
| Compound | NT50 |

BetaCoV OC43 Historical Human Pseudovirus

| | |
|---|---|
| OC43_WT_Prko_AY585228 | 7,556 |
| OC43_S2P_Prko_AY585228 | 2,20,985 |
| OC43_S2P_Prko | 452 |

AlphaCoV 229E Historical Human Pseudovirus

| | |
|---|---|
| 229E_WT_AF304460 | 279,820 |
| 229E_S2P_AF304460 | 102, 571 |
| 229E_S2P | 4,998 |

AlphaCoV NL63 Historical Human Pseudovirus

| | |
|---|---|
| NL63_WT_AY567487 | 17,879 |
| NL63_S2P_AY567487 | 134,626 |
| NL63_S2P | 7,794 |

### Example 7: Historical Pseudovirus Combination Neutralization Studies

These studies examined the neutralization capacity of antibodies resulting from combination of immunogens including BetaCoV (OC43_S2P_Prko, HKU_S2P_Prko) and AlphaCoV (229E_S2P, NL63_S2P) derived mRNA.

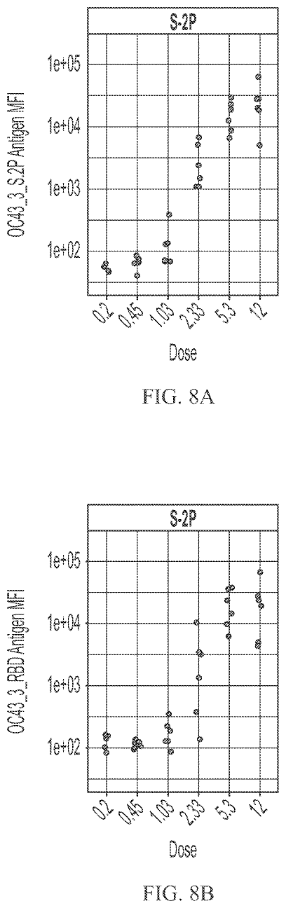

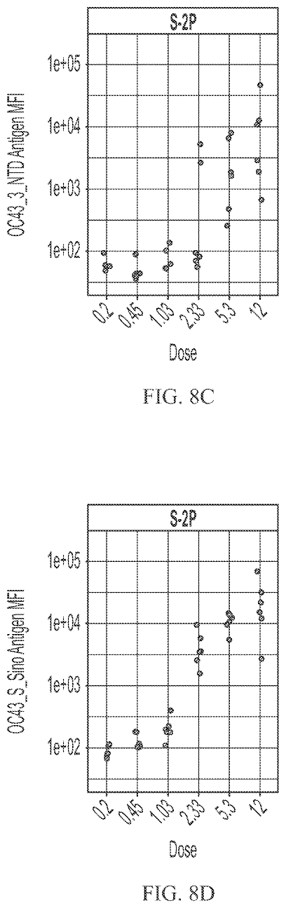

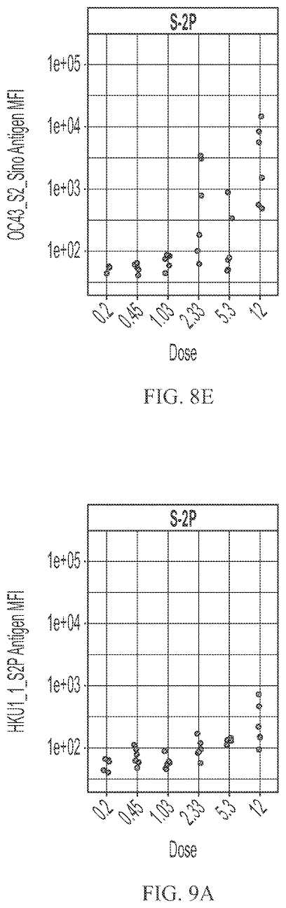

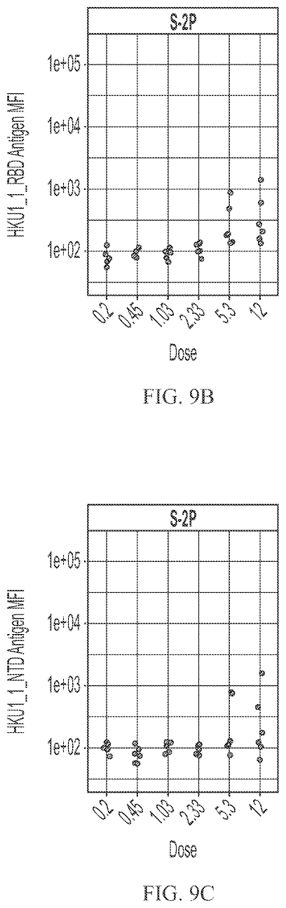

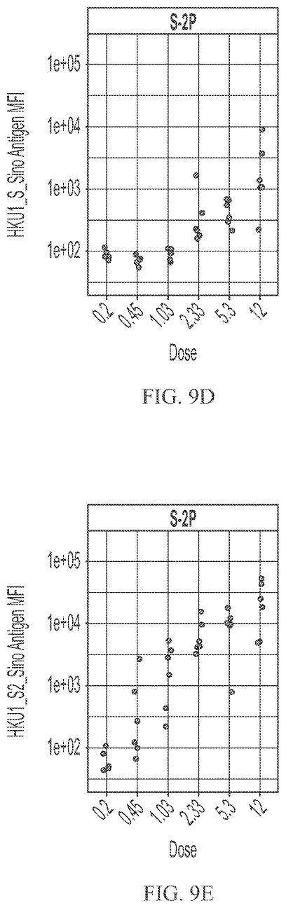

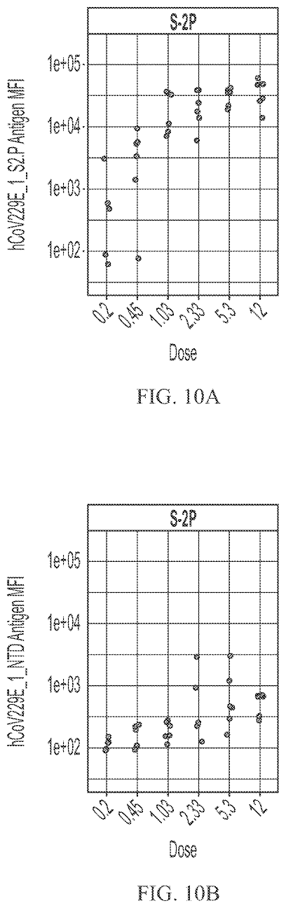

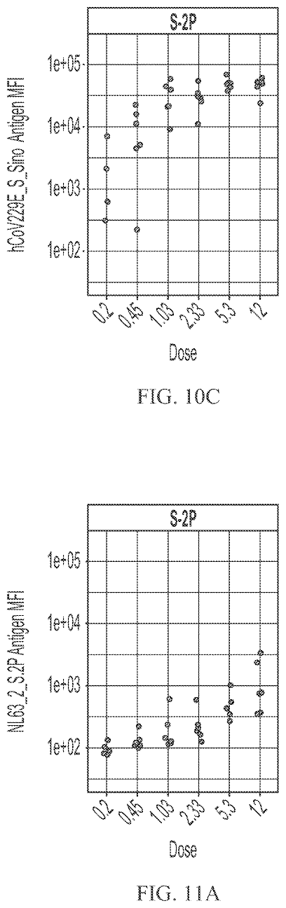

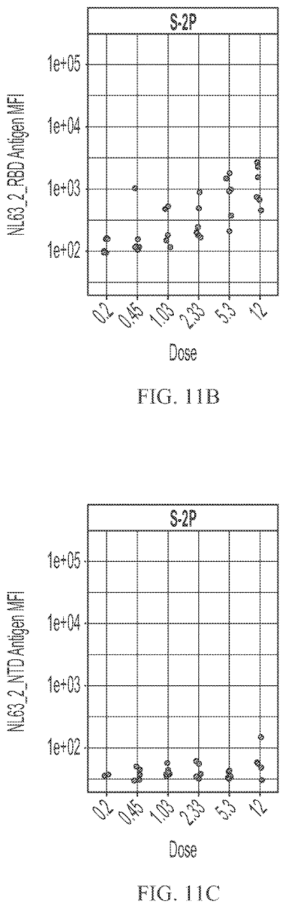

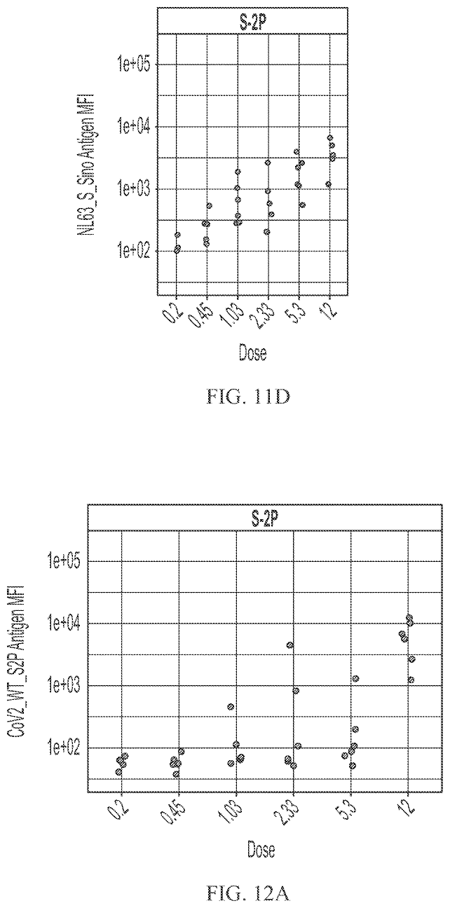

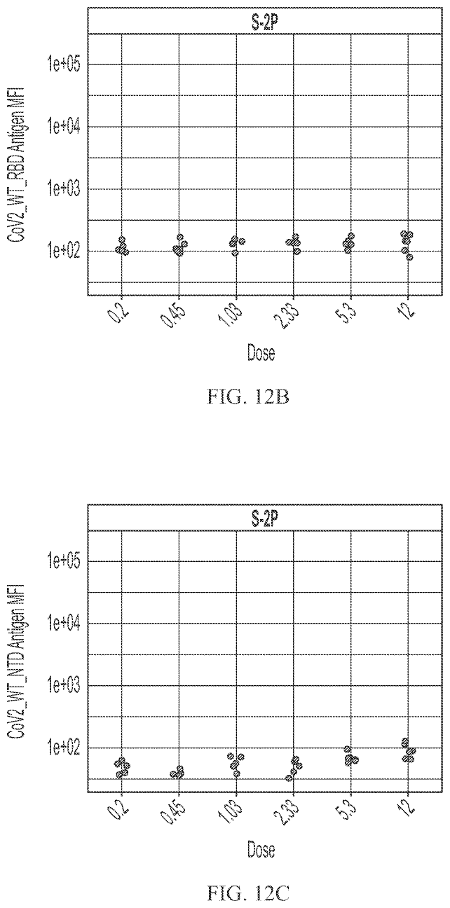

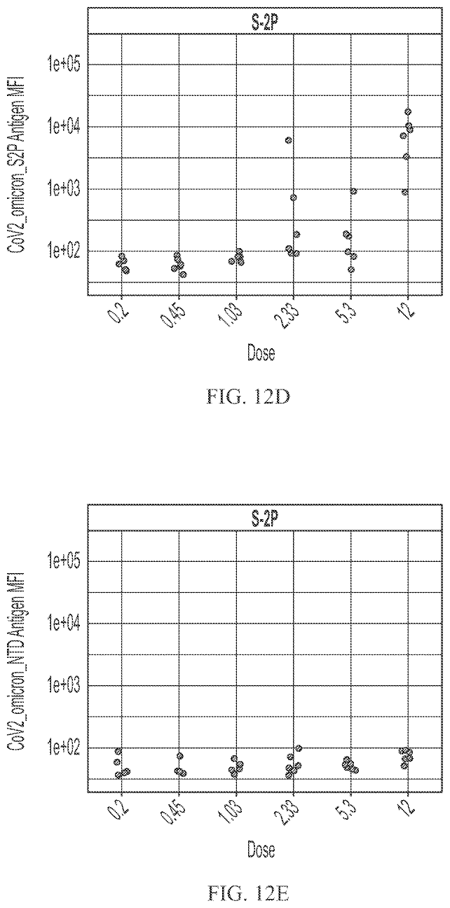

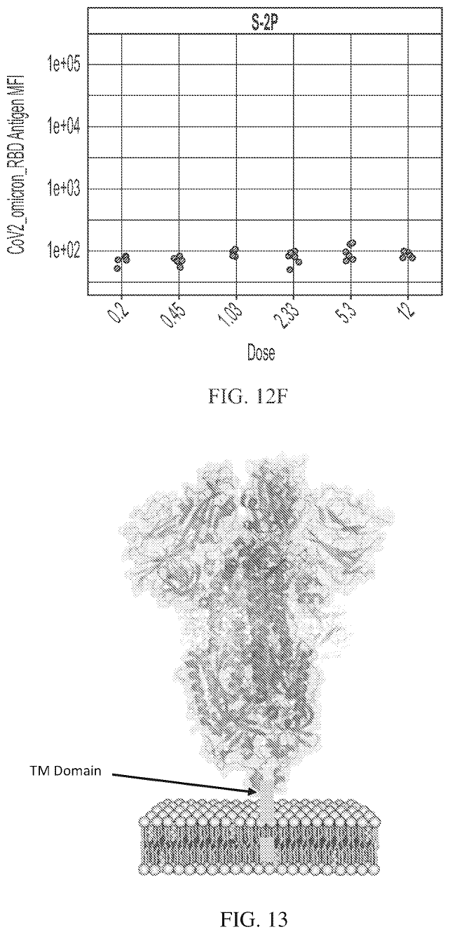

On days 1 and 22, BALB/c mice were immunized intra-muscularly (IM) with 12, 5.3, 2.33, 1.03, 0.45, or 0.20 μg of mRNA encoding a combination of the following proteins: OC43_S2P_Prko, HKU_S2P_Prko, 229_S2P, NL63_S2P at a 1:1:1:1 ratio. The immunization regimen was a prime on day 1 and boost on day 21 with the same amount of specified vaccine (Table 8). On day 36, sera were collected for neutralization and Luminex analysis. Binding was examined for S-2P, RBD antigens, NTD antigens, S antigens, and S2 antigens from OC43 (FIGS. 8A-8E), and HKU (FIGS. 9A-9E) pseudoviruses. Binding was examined for S-2P antigens, NTD antigens, and S antigens from 229E pseudovirus (FIGS. 10A-10C). Binding was also examined for S2-P antigens, RBD antigens, NTD antigens, and S antigens from NL63 (FIGS. 11A-11D) pseudovirus. Antibody binding and neutralization for each pseudovirus tested was found to be present in the mouse samples (as demonstrated by mean fluorescence intensity). OC43, 229E, and NL63 pseudovirus neutralization assays were performed. Briefly, VSVΔG-based pseudoviruses (PsV) were generated for each virus and then cells were infected with each pseudovirus separately and neutralization was measured. Neutralization was observed for each pseudovirus with the combination mRNA formulation. The results are shown in Table 9 below. In particular, neutralization was observed with the VSV PsV expressing OC43-d19 spike protein. In studies with the VSV pseudovirus expressing the 229E-d19 spike protein, neutralizing titers were observed. Further, studies were conducted with the VSV PsV expressing NL63-d19 spike protein and neutralizing titers were observed following combination mRNA exposure.

TABLE 8
mRNA immunogen 1273 Designs

| | | Dose | |
|---|---|---|---|
| Group | Material | (ug/ms) | #Mice |
| 1 | 1283: OC43, HKU1, 229E, NL63 (1:1:1:1) | 12 | 6 |
| 2 | 1283: OC43, HKU1, 229E, NL63 (1:1:1:1) | 5.3 | 6 |
| 3 | 1283: OC43, HKU1, 229E, NL63 (1:1:1:1) | 2.33 | 6 |
| 4 | 1283: OC43, HKU1, 229E, NL63 (1:1:1:1) | 1.03 | 6 |
| 5 | 1283: OC43, HKU1, 229E, NL63 (1:1:1:1) | 0.45 | 6 |
| 6 | 1283: OC43, HKU1, 229E, NL63 (1:1:1:1) | 0.20 | 6 |

TABLE 9
Combination mRNA Treatments Pseudovirus
Neutralization Titer

| | |
|---|---|
| OC43, HKU1, 229E, NL63 Dose | |
| (ug/ms) | NT50 |

VSV-OC43-d19 Spike (USA/1967)

| | |
|---|---|
| 12 | 50,650 |
| 5.3 | 28,455 |
| 2.33 | 42,018 |
| 1.03 | 14,176 |
| 0.45 | 3,381 |
| 0.20 | 1,058 |

VSV-229E-d19 Spike (USA/1973)

| | |
|---|---|
| 12 | 58,966 |
| 5.3 | 63,174 |
| 2.33 | 104,929 |
| 1.03 | 32,577 |
| 0.45 | 5,871 |
| 0.20 | 12,123 |

VSV-NL63-d19 Spike (Amsterdam/2003)

| | |
|---|---|
| 12 | 6,243 |
| 5.3 | 3,880 |
| 2.33 | 3,694 |
| 1.03 | 1,139 |
| 0.45 | 979 |
| 0.20 | 376 |

### SEQUENCE LISTING

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a poly(A) tail and/or cap (e.g., 7 mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 36)
GGGAAAUAAGAGAGAAAAGAAGAGUAAGAAGAAAUAUAAGAGCCACC
5′ UTR:
(SEQ ID NO: 2)
GGGAAAUAAGAGAGAAAAGAAGAGUAAGAAGAAAUAUAAGACCCCGG
CGCCGCCACC
3′ UTR:
(SEQ ID NO: 37)
UGAUAAUAGGCUGGAGCCUCGGUGGCCAUGCUUCUUGCCCCUUGGGC
CUCCCCCCAGCCCCUCCUCCCCUUCCUGCACCCGUACCCCCGUGGUC
UUUGAAUAAAGUCUGAGUGGGCGGC
3′ UTR:
(SEQ ID NO: 4)
UGAUAAUAGGCUGGAGCCUCGGUGGCCUAGCUUCUUGCCCCUUGGGC
CUCCCCCCAGCCCCUCCUCCCCUUCCUGCACCCGUACCCCCGUGGUC
UUUGAAUAAAGUCUGAGUGGGCGGC

TABLE 1
Human Coronaviruses with 2P Mutation
OC43_S2P_Prko

| SEQ ID NO: 1 consists of from 5′ end to 3′ end: 5′ UTR | | 1 |
|---|---|---|
| SEQ ID NO: 2, mRNA ORF SEQ ID NO: 3, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGUUCCUUAUCCUGCUGAUCAGCCUGCCCACCGCCUUCGCCGUG | 3 |
| Construct | AUCGGCGACCUGAAGUGCCCACUGGAGAGCCGGACCGGCAGCCUG | |
| (excluding the | AAUAACAUCGAUACCGGACCGCCCAGCAUCAGCACCGCCACCGUG | |
| stop codon) | GACGUGACCAACGGCCUGGGCACCUACUACGUGCUGGACCGGGUG | |
| | UACCUGAAUACCACCCUGUUCCUGAACGGCUAUUACCCCACCAGC | |
| | GGCAGCACCUACCGGAAUAUGGCGCUGAAGGGCACCGACAAGCUG | |
| | AGCACCCUGUGGUUCAAACCACCCUUCCUGAGCGACUUUAUCAAC | |
| | GGCAUUUUCGCGAAGGUGAAGAACACCAAGGUGUUCAAGGACGGC | |
| | GUGAUGUACAGCGAGUUCCCCGCCAUCACCAUUGGGAGCACCUUC | |
| | GUCAACACCAGCUACUCUGUGGUGGUGCAGCCCCGGACCAUCAAC | |
| | AGCACCCAGGACGGUGUGAACAAGCUGCAGGGCCUGCUGGAGGUG | |
| | AGCGUGUGCCAGUACAACAUGUGCGAGUAUCCCCACACCAUCUGC | |
| | CACCCCAACCUGGGCAACCACUUCAAGGAGCUGUGGCACAUGGAC | |
| | ACCGGAGUGGUGAGCUGCCUGUACAAGCGGAACUUCACCUACGAC | |
| | GUGAACGCUACCUACCUGUACUUCCACUUCUACCAGGAGGGCGGC | |
| | ACCUUCUACGCCUACUUCACCGAUACCGGCGUGGUGACCAAGUUC | |
| | CUGUUCAACGUGUACCUGGGCAUGGCCCUGAGCCACUACUACGUU | |
| | AUGCCCCUGACCUGUAUCAGCCGGCGGAACAUCGGCUUCACCCUG | |
| | GAGUACUGGGUGACCCCUCUGACCAGCCGGCAGUACCUGCUGGCC | |
| | UUCAACCAGGACGGCAUCAUCUUCAACGCCGUGGACUGCAUGUCA | |
| | GACUUCAUGAGCGAGAUCAAGUGCAAGACCCAGAGCAUCGCCCCU | |
| | CCCACCGGCGUGUACGAGCUGAACGGUUACACCGUGCAGCCCAUC | |
| | GCCGACGUGUACCGGCGGAAGCCCGACCUGCCCAACUGCAACAUC | |
| | GAGGCCUGGCUGAACGACAAGAGCGUGCCCAGCCCACUGAACUGG | |
| | GAGCGGAAGACCUUCAGUAACUGCAACUUCAACAUGAGCAGCCUG | |
| | AUGAGCUUCAUCCAGGCCGACUCAUUCACCUGCAACAACAUCGAC | |
| | GCCGCCAAGAUCUACGGCAUGUGCUUCAGCAGCAUCACCAUCGAC | |
| | AAGUUCGCCAUCCCAAACGGCCGGAAGGUGGACUUACAGCUGGGC | |
| | AAUCUGGGCUACCUGCAGAGCUUCAACUACCGGAUCGACACGACC | |
| | GCCACCUCCUGCCAGUUAUACUACAAUCUGCCUGCCGCCAACGUG | |
| | AGCGUGAGCCGCUUCAACCCCAGCACCUGGAACAAGCGGUUCGGC | |
| | UUCAUCGAGGACAGCGUGUUCAAACCCCAGCCCGCCGGCGUGUUC | |
| | ACAAACCACGACGUGGUGUACGCCCAGCACUGCUUUAAGGCCCCU | |
| | AAGAACUUCUGCCCCUGCAAGUCCAAUAGCAGCUUGUGCGUCGGG | |
| | UCUGGCCCCGGAAAGAACAACGGGAUUGGCACCUGCCCCGCUGGC | |
| | ACCAACUACCUGACCUGCCACAACCUCUGCAACCCCGACCCCAUCA | |
| | CCUUCACCGGCCCCUACAAGUGUCCCCAGACCAAGAGCCUGGUGG | |
| | GCAUCGGCGAGCACUGCAGCGGCCUGGCAGUGAAGAGCGACUACU | |
| | GCGGCGGCAACCUGUGCACCUGCCAGCCCCAGGCCUUCCUGGGCUG | |
| | GAGCGCCGACAGCUGCCUGCAGGGCGACAAGUGCAACAUCUUCGC | |
| | CAAUCUGAUCCUGCACGACGUCAACAGCGGACUGACCUGCAGCAC | |
| | CGACCUGCAGAAGGCCAACACCGACAUCAAGCUGGGCGUGUGCGU | |
| | GAACUACGACCUGUACGGCAUCAGCGGCCAGGGCAUCUUCGUGGA | |
| | GGUCAACGCCACCUAUUACAACAGCUGGCAGAACCUGCUGUACGA | |
| | CAGCAACGGUAAUUUAUACGGCUUCCGGGACUACAUCACCAACCG | |
| | GACCUUCAUGAUCCGGAGCUGCUACAGCGGCCGGGUGAGCGCAGC | |
| | UUUCCACGCCAACAGCAGCGAGCCCGCCCUGCUGUUUCGGAACAU | |
| | CAAGUGUAACUACGUGUUCAACAACAGCCUGACCCGGCAGCUGCA | |
| | GCCUAUCAACUACUUCGACAGCUACCUGGGCUGCGUGGUGAACGC | |
| | AUACAACAGCACUGCCAUCAGCGUGCAGACCUGCGACCUGACCGU | |
| | GGGCUCCGGGUACUGCGUGGACUACAGCAAGAACGGCGGCUCCGG | |
| | CGGAGCUAUCACCACCGGCUACCGGUUCACCAACUUCGAGCCCUUC | |
| | ACCGUGAACAGCGUGAACGACAGCCUGGAGCCCGUGGGCGGACUG | |
| | UACGAGAUCCAGAUCCCCAGCGACUUCACCAUCGGCAACAUGGAG | |
| | GAGUUCAUCCAGACCUCAAGCCCCAAGGUGACCAUCGACUGCGCC | |
| | GCCUUCGUGUGCGGCGACUACGCCGCCUGCAAGAGCCAGCUGGUG | |
| | GAGUACGGCAGCUUCUGCGACAACAUCAACGCCAUCCUGACCGAG | |
| | GUGAACGAACUGCUGGACACCACCCAGUUACAGGUGGCCAACAGU | |
| | CUGAUGAACGGCGUGACCCUGAGUAUCAAGCUGAAGGACGGAGUG | |
| | AACUUCAACGUGGACGACAUCAAUUUCAGCAGCGUGCUGGGCUGC | |
| | CUGGGCAGCGAGUGCAGCAAGGCCAGUAGCCGGAGCGCCAUAGAG | |
| | GACCUGCUGUUCGACAAGGUGAAACUGAGCGACGUGGGCUUCGUG | |
| | GCCGCCUACAACAACUGCACCGGUGGCGCCGAGAUCCGGGACCUG | |
| | AUCUGCGUGCAGAGCUACAAGGGCAUCAAAGUGCUGCCACCUCUG | |
| | CUGAGCGAGAACCAGAUCAGCGGCUACACCCUCGCCGCCACCAGCG | |
| | CUAGCCUGUUUCCUCCUUGGACUGCUGCCGCAGGCGUGCCCUUCU | |
| | ACCUGAACGUGCAGUACCGGAUUAACGGCCUCGGUGUGACCAUGG | |
| | ACGUGCUGAGCCAGAACCAGAAGCUGAUCGCCAACGCCUUCAACA | |
| | ACGCCCUGGACGCCAUCCAGGAGGGAUUCGACGCCACCAACAGCG | |
| | CCCUGGUGAAGAUCCAGGCGGUAGUGAACGCCAACGCUGAGGCCC | |
| | UGAACAACCUGCUGCAACAGCUGAGCAACCGGUUCGGCGCCAUCA | |
| | GCAGCAGCCUGCAGGAGAUCCUGAGCCGGCUGGAUCCACCAGAGG | |
| | CCGAGGCCCAGAUCGACCGGCUGAUCAACGGCCGGCUGACCGCCCU | |
| | GAACGCCUACGUGAGCCAGCAGCUGAGCGACAGCACCCUGGUCAA | |
| | GUUCAGCGCCGCCCAGGCCAUGGAGAAAGUGAACGAGUGCGUGAA | |
| | GAGCCAGAGCAGCCGGAUCAACUUCUGCGGCAACGGCAACCACAU | |
| | CAUCAGCCUGGUGCAGAACGCCCCUUACGGCCUGUACUUUAUCCA | |
| | CUUCAGCUACGUGCCCACCAAGUACGUGACCGCCAAGGUGAGCCC | |
| | CGGCCUGUGCAUCGCCGGCGACCGGGGAAUCGCUCCCAAGAGCGG | |
| | CUACUUCGUGAACGUGAACAACACCUGGAUGUACACCGGCAGCGG | |
| | AUACUACUACCCCGAGCCCAUCACCGAGAACAACGUGGUGGUGAU | |
| | GAGCACCUGCGCAGUGAACUACACCAAGGCUCCUUACGUGAUGCU | |
| | GAACACCUCUAUCCCAAAUCUGCCCGACUUCCGGGAGGAGCUGGA | |
| | CCAGUGGUUCAAGAACCAGACCAGCGUGGCCCCUGAUCUGAGCCU | |
| | GGACUACAUUAACGUGACCUUCCUGGACCUGCAGGUGGAGAUGAA | |
| | CCGGCUGCAGGAGGCCAUCAAGGUGCUGAACCAGUCAUACAUCAA | |
| | CCUGAAGGACAUCGGCACCUACGAGUACUACGUGAAGUGGCCCUG | |
| | GUACGUGUGGCUGCUGAUCUGCCUGGCCGGCGUGGCCAUGCUGGU | |
| | GCUGCUGUUCUUCAUCUGCUGCUGCACCGGCUGCGGCACCAGCUG | |
| | CUUCAAGAAGUGCGGCGGCUGCUGCGACGACUACACCGGAUACCA | |
| | GGAGCUGGUGAUCAAGACCAGCCACGACGAC | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MFLILLISLPTAFAVIGDLKCPLESRTGSLNNIDTGPPSISTATVDVTNGLG | 5 |
| amino acid | TYYVLDRVYLNTTLFLNGYYPTSGSTYRNMALKGTDKLSTLWFKPPFL | |
| sequence | SDFINGIFAKVKNTKVFKDGVMYSEFPAITIGSTFVNTSYSVVVQPRTINS | |
| | TQDGVNKLQGLLEVSVCQYNMCEYPHTICHPNLGNHFKELWHMDTGV | |
| | VSCLYKRNFTYDVNATYLYFHFYQEGGTFYAYFTDTGVVTKFLFNVYL | |
| | GMALSHYYVMPLTCISRRNIGFTLEYWVTPLTSRQYLLAFNQDGIIFNA | |
| | VDCMSDFMSEIKCKTQSIAPPTGVYELNGYTVQPIADVYRRKPDLPNCN | |
| | IEAWLNDKSVPSPLNWERKTFSNCNFNMSSLMSFIQADSFTCNNIDAAK | |
| | IYGMCFSSITIDKFAIPNGRKVDLQLGNLGYLQSFNYRIDTTATSCQLYY | |
| | NLPAANVSVSRFNPSTWNKRFGFIEDSVFKPQPAGVFTNHDVVYAQHC | |
| | FKAPKNFCPCKSNSSLCVGSGPGKNNGIGTCPAGTNYLTCHNLCNPDPI | |
| | TFTGPYKCPQTKSLVGIGEHCSGLAVKSDYCGGNLCTCQPQAFLGWSA | |
| | DSCLQGDKCNIFANLILHDVNSGLTCSTDLQKANTDIKLGVCVNYDLY | |
| | GISGQGIFVEVNATYYNSWQNLLYDSNGNLYGFRDYITNRTFMIRSCYS | |
| | GRVSAAFHANSSEPALLFRNIKCNYVFNNSLTRQLQPINYFDSYLGCVV | |
| | NAYNSTAISVQTCDLTVGSGYCVDYSKNggsggAITTGYRFTNFEPFTVNS | |
| | VNDSLEPVGGLYEIQIPSDFTIGNMEEFIQTSSPKVTIDCAAFVCGDYAA | |
| | CKSQLVEYGSFCDNINAILTEVNELLDTTQLQVANSLMNGVTLSIKLKD | |
| | GVNFNVDDINFSSVLGCLGSECSKASSRSAIEDLLFDKVKLSDVGFVAA | |
| | YNNCTGGAEIRDLICVQSYKGIKVLPPLLSENQISGYTLAATSASLFPPW | |
| | TAAAGVPFYLNVQYRINGLGVTMDVLSQNQKLIANAFNNALDAIQEGF | |
| | DATNSALVKIQAVVNANAEALNNLLQQLSNRFGAISSSLQEILSRLDPPE | |
| | AEAQIDRLINGRLTALNAYVSQQLSDSTLVKFSAAQAMEKVNECVKSQ | |
| | SSRINFCGNGNHIISLVQNAPYGLYFIHFSYVPTKYVTAKVSPGLCIAGD | |
| | RGIAPKSGYFVNVNNTWMYTGSGYYYPEPITENNVVVMSTCAVNYTK | |
| | APYVMLNTSIPNLPDFREELDQWFKNQTSVAPDLSLDYINVTFLDLQVE | |
| | MNRLQEAIKVLNQSYINLKDIGTYEYYVKWPWYVWLLICLAGVAMLV | |
| | LLFFICCCTGCGTSCFKKCGGCCDDYTGYQELVIKTSHDD | |
| PolyA tail | 100 nt | |

HKU1_S2P_Prko

| SEQ ID NO: 6 consists of from 5′ end to 3′ end: 5′ UTR | | 6 |
|---|---|---|
| SEQ ID NO: 2, mRNA ORF SEQ ID NO: 7, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGCUGCUGAUCAUCUUUAUCCUGCCCACCACCCUGGCCGUGAUC | 7 |
| Construct | GGAGACUUCAACUGUACCAACUUCGCUAUUAACGACCUGAACACA | |
| (excluding the | ACCAUACCCCGGAUCAGCGAGUACGUGGUGGACGUGAGCUACGGU | |
| stop codon) | CUGGGCACCUACUACAUCCUGGACCGGGUGUACCUGAAUACCACC | |
| | AUCCUGUUUACCGGCUACUUCCCCAAGAGCGGCGCCAACUUCCGG | |
| | GACCUGAGCCUGAAGGGCACCACCAAGCUGAGCACCCUGUGGUAC | |
| | CAGAAGCCCUUCCUCUCCGACUUCAAUAACGGCAUCUUCAGCCGG | |
| | GUGAAGAAUACCAAGCUAUACGUGAACAAGACCCUGUACAGCGAG | |
| | UUCAGCACCAUAGUGAUCGGCAGCGUGUUUAUCAACAAUAGCUAU | |
| | ACAAUCGUGGUGCAGCCCCACAACGGCGUGCUGGAGAUCACAGCC | |
| | UGCCAGUACACCAUGUGCGAGUAUCCCCACACCAUCUGCAAGUCA | |
| | AUUGGCAGCUCCCGGAACGAGAGCUGGCACUUCGAUAAGAGCGAG | |
| | CCCCUGUGCCUAUUCAAGAAGAACUUUACUUACAACGUGAGCACC | |
| | GAUUGGCUGUACUUCCACUUCUACCAGGAGCGGGGCACCUUCUAC | |
| | GCCUACUACGCAGACAGCGGCAUGCCCACUACCUUCCUGUUCAGC | |
| | UUAUACCUGGGAACCCUGCUAAGCCACUAUUACGUGCUGCCCCUG | |
| | ACCUGCAACGCCAUCUCGAGCAACACCGACAACGAGACCCUGCAG | |
| | UACUGGGUGACCCCUCUGAGCAAGCGGCAGUACCUGCUGAAAUUC | |
| | GACAACCGGGGCGUGAUCACCAACGCCGUGGACUGCAGUAGCAGC | |
| | UUCUUUAGCGAGAUCCAGUGCAAGACCAAGAGCCUGCUGCCCAAC | |
| | ACCGGCGUGUACGAUCUGAGCGGAUUUACCGUGAAGCCCGUGGCC | |
| | ACCGUGCACCGGCGGAUCCCCGACCUGCCUGACUGCGACAUCGACA | |
| | AGUGGCUGAAUAACUUUAACGUGCCCAGUCCUCUGAACUGGGAGC | |
| | GGAAGAUUUUCAGUAACUGCAACUUUAAUCUGUCCACCCUGCUGC | |
| | GGCUGGUGCACACCGAUAGCUUUAGCUGCAACAACUUCGACGAGA | |
| | GCAAGAUCUACGGCAGCUGCUUCAAGAGCAUCGUGCUCGAUAAGU | |
| | UCGCCAUCCCCAACAGCCGGCGGAGCGACCUGCAGCUGGGCAGUA | |
| | GCGGAUUCCUGCAGAGCAGCAAUUACAAGAUCGACACCACCAGCA | |
| | GCUCUUGCCAGUUGUACUACAGCCUACCCGCCAUCAACGUGACAA | |
| | UCAACAACUACAACCCUAGCAGCUGGAACCGGCGGUACGGCUUUA | |
| | ACAAUUUUAACCUGAGCAGUCACAGUGUGGUGUACAGCCGGUACU | |
| | GCUUCAGUGUGAACAACACCUUUUGCCCCUGCGCCAAGCCCAGCU | |
| | UCGCCAGCUCGUGCAAGAGCCACAAGCCACCUAGCGCCAGCUGCCC | |
| | CAUCGGCACCAACUACCGGAGCUGCGAGAGCACCACAGUGCUGGA | |
| | CCACACCGACUGGUGCCGGUGUAGCUGCCUGCCCGACCCCAUCACC | |
| | GCCUACGACCCUCGGAGCUGUAGCCAGAAGAAGUCUCUGGUGGGC | |
| | GUGGGCGAGCACUGCGCCGGCUUCGGCGUGGACGAGGAGAAGUGC | |
| | GGCGUACUGGACGGGAGCUAUAACGUGAGUUGCCUGUGUAGCACC | |
| | GACGCCUUCCUGGGGUGGAGCUACGACACCUGCGUGAGCAACAAC | |
| | CGGUGCAACAUAUUCAGCAAUUUCAUCCUGAACGGAAUUAACUCU | |
| | GGCACCACUUGCAGUAACGAUCUGCUGCAGCCCAAUACGGAGGUG | |
| | UAUACCGACGUGUGCGUUGACUACGACUUGUACGGCAUCACCGGA | |
| | CAGGGCAUCUUUAAGGAGGUGUCGGCCGUGUACUAUAACUCUUGG | |
| | CAGAACCUGCUCUACGACUCCAACGGCAACAUCAUCGGUUUCAAG | |
| | GACUUUGUGACCAACAAGACAUACAACAUCUUUCCCUGCUACGCU | |
| | GGUCGGGUGAGCGCCGCGUUCCACCAGAACGCCAGCAGCCUGGCU | |
| | CUGCUGUACCGGAACCUCAAGUGCAGCUACGUGCUCAACAACAUU | |
| | AGCCUGGCCACCCAGCCCUACUUCGACAGCUACCUGGGCUGCGUG | |
| | UUCAACGCCGACAACCUGACCGACUACAGCGUGUCAAGCUGCGCC | |
| | CUGCGGAUGGGCAGCGGCUUCUGCGUGGACUAUAACAGCCCCAGC | |
| | UUUUCCAGCAGCGGCGGCUCCGGUGGUAGCAUCUCCGCCAGCUAC | |
| | CGGUUCGUGACCUUCGAGCCCUUCAACGUCAGCUUCGUGAACGAC | |
| | AGCAUCGAGAGUGUGGGCGGUCUGUACGAGAUCAAGAUCCCCACC | |
| | AACUUUACCAUCGUGGGCCAGGAGGAGUUCAUCCAGACCAACAGU | |
| | CCCAAGGUGACCAUCGACUGCAGCCUGUUCGUGUGCAGCAACUAC | |
| | GCCGCCUGCCACGACCUGUUAUCGGAGUACGGUACCUUCUGCGAC | |
| | AAUAUCAACAGCAUCUUAGACGAAGUGAACGGCCUGCUGGACACC | |
| | ACUCAGCUGCACGUGGCUGACACCCUGAUGCAAGGCGUGACUCUG | |
| | UCUAGCAACCUUAACACCAACCUGCAUUUCGACGUGGACAACAUC | |
| | AAUUUCAAGUCCCUGGUGGGGUGCCUGGGGCCUCACUGCGGCUCA | |
| | AGCAGCCGGAGCUUCUUCGAGGACCUGUUGUUCGACAAGGUGAAG | |
| | CUGUCCGACGUGGGCUUCGUGGAGGCCUACAACAACUGCACUGGU | |
| | GGCUCCGAGAUCCGGGACUUACUGUGCGUGCAGAGCUUCAACGGC | |
| | AUUAAGGUGCUGCCUCCCAUCCUGAGCGAGAGCCAAAUCAGCGGC | |
| | UACACCACCGCCGCCACGGUUGCUGCCAUGUUUCCGCCUUGGAGC | |
| | GCUGCGGCCGGGAUACCCUUCAGCCUGAACGUGCAGUACAGAAUC | |
| | AACGGACUGGGAGUGACCAUGGACGUGUUGAAUAAGAAUCAGAAG | |
| | CUGAUCGCCACCGCCUUCAACAACGCCCUGCUGAGCAUCCAGAACG | |
| | GCUUCAGCGCCACCAACAGCGCCCUGGCCAAGAUCCAGAGCGUGG | |
| | UGAACAGCAACGCCCAGGCCCUGAAUAGCCUGCUACAGCAGCUGU | |
| | UCAACAAGUUCGGGGCCAUCUCUUCCAGCCUGCAGGAGAUCCUGU | |
| | CUCGGCUGGACCCUCCUGAGGCCCAGGUGCAGAUCGACCGGCUGA | |
| | UCAACGGCCGGCUGACCGCCCUGAACGCCUACGUGAGCCAGCAAC | |
| | UGAGCGAUAUCAGCCUGGUGAAGUUCGGUGCCGCUCUGGCCAUGG | |
| | AGAAGGUGAACGAGUGCGUGAAGAGCCAGAGCCCACGGAUCAACU | |
| | UCUGCGGAAACGGUAACCACAUCCUGUCACUGGUGCAGAACGCUC | |
| | CCUACGGCCUUCUGUUCAUGCACUUCAGCUACAAGCCCAUCUCAU | |
| | UCAAGACCGUGCUGGUGAGCCCCGGCCUGUGCAUCAGCGGGGACG | |
| | UGGGAAUCGCGCCCAAGCAGGGCUACUUUAUCAAGCACAACGACC | |
| | ACUGGAUGUUCACCGGCAGCAGCUACUACUACCCCGAGCCCAUCA | |
| | GCGACAAGAACGUGGUGUUCAUGAAUACUUGCAGCGUGAAUUUCA | |
| | CCAAGGCCCCGCUGGUGUAUCUGAACCACAGCGUGCCCAAGCUGU | |
| | CAGACUUCGAAAGCGAGCUGAGCCACUGGUUCAAGAACCAGACCA | |
| | GCAUCGCCCCAAACCUGACACUGAACCUGCACACCAUUAACGCCAC | |
| | CUUUCUGGACCUGUACUACGAAAUGAACCUGAUCCAGGAGAGCAU | |
| | CAAGAGCUUAAACAACAGCUACAUCAACCUGAAGGAUAUCGGCAC | |
| | AUACGAGAUGUACGUGAAGUGGCCCUGGUACGUGUGGCUGCUCAU | |
| | AAGCUUCAGCUUCAUCAUCUUCCUGGUGCUGCUGUUCUUCAUCUG | |
| | CUGUUGCACGGGCUGCGGCAGCGCUUGCUUCAGCAAGUGCCACAA | |
| | CUGCUGCGACGAGUACGGCGGCCACCACGACUUCGUGAUCAAGAC | |
| | CAGCCACGACGAC | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGGGC | |
| Corresponding | MLLIIFILPTTLAVIGDFNCTNFAINDLNTTIPRISEYVVDVSYGLGTYYIL | 8 |
| amino acid | DRVYLNTTILFTGYFPKSGANFRDLSLKGTTKLSTLWYQKPFLSDENNGI | |
| sequence | FSRVKNTKLYVNKTLYSEFSTIVIGSVFINNSYTIVVQPHNGVLEITACQY | |
| | TMCEYPHTICKSIGSSRNESWHFDKSEPLCLFKKNFTYNVSTDWLYFHF | |
| | YQERGTFYAYYADSGMPTTFLFSLYLGTLLSHYYVLPLTCNAISSNTDN | |
| | ETLQYWVTPLSKRQYLLKFDNRGVITNAVDCSSSFFSEIQCKTKSLLPNT | |
| | GVYDLSGFTVKPVATVHRRIPDLPDCDIDKWLNNFNVPSPLNWERKIFS | |
| | NCNFNLSTLLRLVHTDSFSCNNFDESKIYGSCFKSIVLDKFAIPNSRRSDL | |
| | QLGSSGFLQSSNYKIDTTSSSCQLYYSLPAINVTINNYNPSSWNRRYGFN | |
| | NFNLSSHSVVYSRYCFSVNNTFCPCAKPSFASSCKSHKPPSASCPIGTNY | |
| | RSCESTTVLDHTDWCRCSCLPDPITAYDPRSCSQKKSLVGVGEHCAGFG | |
| | VDEEKCGVLDGSYNVSCLCSTDAFLGWSYDTCVSNNRCNIFSNFILNGI | |
| | NSGTTCSNDLLQPNTEVYTDVCVDYDLYGITGQGIFKEVSAVYYNSWQ | |
| | NLLYDSNGNIIGFKDFVTNKTYNIFPCYAGRVSAAFHQNASSLALLYRN | |
| | LKCSYVLNNISLATQPYFDSYLGCVFNADNLTDYSVSSCALRMGSGFCV | |
| | DYNSPSFSSSGGSGGSISASYRFVTFEPFNVSFVNDSIESVGGLYEIKIPTN | |
| | FTIVGQEEFIQTNSPKVTIDCSLFVCSNYAACHDLLSEYGTFCDNINSILD | |
| | EVNGLLDTTQLHVADTLMQGVTLSSNLNTNLHFDVDNINFKSLVGCLG | |
| | PHCGSSSRSFFEDLLFDKVKLSDVGFVEAYNNCTGGSEIRDLLCVQSFN | |
| | GIKVLPPILSESQISGYTTAATVAAMFPPWSAAAGIPFSLNVQYRINGLG | |
| | VTMDVLNKNQKLIATAFNNALLSIQNGFSATNSALAKIQSVVNSNAQA | |
| | LNSLLQQLFNKFGAISSSLQEILSRLDPPEAQVQIDRLINGRLTALNAYVS | |
| | QQLSDISLVKFGAALAMEKVNECVKSQSPRINFCGNGNHILSLVQNAPY | |
| | GLLFMHFSYKPISFKTVLVSPGLCISGDVGIAPKQGYFIKHNDHWMFTG | |
| | SSYYYPEPISDKNVVFMNTCSVNFTKAPLVYLNHSVPKLSDFESELSHW | |
| | FKNQTSIAPNLTLNLHTINATFLDLYYEMNLIQESIKSLNNSYINLKDIGT | |
| | YEMYVKWPWYVWLLISFSFIIFLVLLFFICCCTGCGSACFSKCHNCCDE | |
| | YGGHHDFVIKTSHDD | |
| PolyA tail | 100 nt | |

229E_S2P

| SEQ ID NO: 9 consists of from 5′ end to 3′ end: 5′ UTR | | 9 |
|---|---|---|
| SEQ ID NO: 2, mRNA ORF SEQ ID NO: 10, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGUUCGUGCUGCUGGUGGCCUACGCCCUGCUGCACAUCGCCGGC | 10 |
| Construct | UGCCAGACCAUUAACGGCACAAACACCAGCCACUCCGUUUGCAAC | |
| (excluding the | GGCUGCGUGGGCCACAGCGAGAACGUCUUCGCCGUGGAGAGCGGC | |
| stop codon) | GGCUACAUCCCUAGCAACUUCGCCUUCAACAACUGGUUCCUGCUG | |
| | ACCAACACCAGCUCCGUGGUGGACGGCGUGGUGCGGAGCUUCCAG | |
| | CCCCUGCUGCUGAACUGCCUGUGGAGUGUGAGCGGCAGCCAGUUC | |
| | AUCACCGGCUUCGUGUACUUCAACGGCACCGGCAAGGGCGCCUGC | |
| | AAGGGCUUCUACAGCAACGCCAGCUCUGACGUGAUUCGGUACAAC | |
| | AUAAACUUCGAGGAGAACCUGCGGCGGGGCACCAUCCUGUUCAAG | |
| | ACCAGCUACGGCGCCGUGGUGUUCUACUGCACCAACAACACCCUG | |
| | GUGAGCGGCGACGCCCACAUCCCCAGCGGCACCGUGCUGGGCAAC | |
| | UUCUACUGCUUCGUGAACACCACCAUCGGCAACGAGACGACCAGC | |
| | GCCUUCGUGGGCGCCCUGCCCAAGACCGUGCGGGAGUUCGUGAUC | |
| | AGCCGGACCGGCCACUUCUACAUUAACGGAUACCGGUACUUCUCC | |
| | CUGGGCAACGUGGAGGCCGUGAACUUUAACGUGACCAACGCCGCC | |
| | ACCACCGUGUGCACCGUAGCCCUGGCCAGCUACGCCGACGUGCUCG | |
| | UAAACGUGAGCCAGACCGCCAUCGCCAACAUCAUCUACUGCAACA | |
| | GCGUGAUCAAUCGGCUGCGGUGCGACCAGCUGAGCUUCGACGUGC | |
| | CCGACGGCUUCUACUCUACCAGCCCCAUCCAGCCUGUGGAACUGCC | |
| | CGUGAGCAUCGUGAGCCUGCCCGUGUACCACAAGCACACCUUCAU | |
| | CGUGCUGUACGUGAACUUCGAGCACCAGAGAGGCCCCGGCAAGUG | |
| | CUACAACUGCCGGCCCGCCGUGAUCAACAUCACCCUGGCUAACUUC | |
| | AACGAGACCAAGGGCCCUCUGUGCGUUGAUACCAGCCAUUUCACC | |
| | ACCCAGUUCGUGGACAACGUCAAGCUGGCCCGGUGGAGCGCCAGC | |
| | AUCGACACCGGCAACUGCCCCUUCAGCUUCGGCAAGGUGAUCAAC | |
| | UUCGUGAAGUUCGGCAGCGUGUGCUUCAGCCUGAAGUACAUCCCA | |
| | GGCGGCUGCGCCAUGCCCAUCAUGGCCAACCUGGUGAACUACAAG | |
| | AGCCACAACAUCGGCAGCUUAUACGUGAGCUGGAGCGACGGCGAC | |
| | GUGAUCACCGGCGUGCCCAAGCCCGUGGAGGGCGUGUCUAGCUUC | |
| | AUGAACGUGACCCUGAACAAGUGCACUAAGUACAACAUCUACGAC | |
| | GUCAGCGGCGUGGGCGUGAUACGGAUCAGCAACGACACCUUCCUG | |
| | AACGGCAUCACCUACACCAGCACCAGCGGCAACCUGCUGGGCUUC | |
| | AAGGACGUGACGAACGGCACCAUCUACAGCAUCACCCCUUGCAAC | |
| | CCUCCUGACCAGCUGGUGGUGUACCAGCAGGCCGUGGUGGGCGCC | |
| | AUGUUAAGCGAGAACUUCACCUCUUACGGCUUCAGCAACGUGGUG | |
| | GAGAUGCCCAAGUUCUUCUACGCCAGCAACGGCACUUACAACUGC | |
| | ACCGACGCCGUGCUGACCUACUCAAGCUUCGGCGUGUGCGCCGAC | |
| | GGCAGCAUCAUCGCCGUGCAGCCCCGGAACGUGAGCUACGACAGC | |
| | GUGAGCGCCAUCGUGACCGCCAACCUGUCUAUCCCCAGCAACUGG | |
| | ACCACCAGCGUGCAGGUGGAGUACCUGCAGAUUACCAGCACUCCC | |
| | AUCGUGGUGGAUUGCAGCACCUACGUGUGCAACGGCAACGUGCGG | |
| | UGCGUGGAGCUGCUGAAGCAGUACACCAGCGCUUGCAAGACCAUC | |
| | GAGGACGCCCUGCGGAACAGCGCCAUGCUGGAGUCUGCCGACGUG | |
| | AGCGAGAUGCUGACCUUCGACAAGAAGGCCUUCACACUGGCCAAC | |
| | GUGAGCAGCUUUGGCGACUACAACCUGAGCAGCGUGAUUCCAAGC | |
| | CUUCCUCGGAGCGGCUCUAGGGUGGCCGGCCGGUCUGCAAUCGAG | |
| | GACAUCUUGUUCAGCAAGCUGGUGACCAGCGGCCUGGGCACAGUG | |
| | GACGCCGACUACAAGAAGUGCACCAAAGGCCUGAGCAUCGCCGAU | |
| | CUGGCCUGCGCCCAGUACUACAACGGAAUCAUGGUGCUUCCAGGG | |
| | GCUGCCGACGCAGAGCGGAUGGCCAUGUACACCGGCAGCCUGAUC | |
| | GGCGGAAUCGCCCUGGGCGGACUGACCAGCGCUGCCAGCAUCCCC | |
| | UUUAGCCUGGCCAUCCAGAGCCGGCUGAACUACGUGGCCCUUCAG | |
| | ACUGACGUGCUGCAGGAGAACCAGCGGAUCCUGGCCGCCAGCUUC | |
| | AACAAGGCCAUGACCAACAUCGUGGACGCCUUCACAGGCGUGAAC | |
| | GACGCCAUCACCCAGACCAGCCAGGCCCUGCAGACCGUGGCCACCG | |
| | CCCUCAACAAGAUCCAGGACGUGGUGAACCAGCAGGGCAACAGCC | |
| | UGAACCACCUGACCUCUCAGCUGCGGCAGAACUUCCAGGCCAUCA | |
| | GCAGUAGCAUCCAAGCAAUCUACGACCGGCUGGAUCCACCACAGG | |
| | CCGACCAGCAGGUGGACCGGCUGAUAACCGGCCGCCUGGCUGCCC | |
| | UUAACGUGUUCGUGAGCCAUACCCUGACCAAGUACACCGAGGUGC | |
| | GGGCCUCUCGGCAGCUGGCCCAGCAGAAGGUGAACGAGUGCGUGA | |
| | AGAGCCAGAGCAAGCGGUACGGCUUCUGCGGCAACGGGACCCACA | |
| | UCUUCAGCCUUGUGAACGCCGCCCCUGAAGGCCUGGUGUUCCUGC | |
| | ACACCGUGCUACUGCCCACCCAGUACAAGGACGUUGAGGCCUGGA | |
| | GCGGCCUGUGCGUGGACGGGAUCAACGGCUACGUGCUCCGGCAGC | |
| | CCAACCUGGCCCUGUACAAGGAGGGCAACUACUACCGGAUCACCA | |
| | GCCGGAUCAUGUUCGAGCCCCGGAUCCCCACCAUCGCCGACUUCGU | |
| | GCAGAUCGAGAACUGCAACGUGACCUUUGUGAACAUUAGCCGGAG | |
| | CGAGCUGCAGACCAUCGUGCCCGAGUACAUCGACGUGAACAAGAC | |
| | CCUGCAGGAGCUGAGCUACAAGCUGCCCAACUACACCGUUCCCGA | |
| | CCUGGUGGUGGAGCAGUACAACCAGACCAUCCUGAACCUGACAAG | |
| | CGAGAUAAGCACCCUGGAGAACAAGAGCGCCGAGCUGAAUUACAC | |
| | CGUGCAGAAGCUCCAGACACUGAUCGACAACAUCAACAGCACUCU | |
| | GGUGGACCUGAAGUGGCUGAACCGGGUGGAGACCUACAUCAAGUG | |
| | GCCCUGGUGGGUGUGGCUGUGCAUCAGCGUGGUGCUGAUCUUCGU | |
| | GGUGAGCAUGCUGCUGCUGUGCUGCUGCAGCACCGGCUGCUGCGG | |
| | CUUCUUCAGCUGCUUCGCCAGCAGCAUCCGGGGUUGCUGCGAGAG | |
| | CACCAAGUUACCCUACUACGACGUGGAGAAGAUCCACAUCCAG | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MFVLLVAYALLHIAGCQTINGTNTSHSVCNGCVGHSENVFAVESGGYIP | 11 |
| amino acid | SNFAFNNWFLLTNTSSVVDGVVRSFQPLLLNCLWSVSGSQFITGFVYFN | |
| sequence | GTGKGACKGFYSNASSDVIRYNINFEENLRRGTILFKTSYGAVVFYCTN | |
| | NTLVSGDAHIPSGTVLGNFYCFVNTTIGNETTSAFVGALPKTVREFVISR | |
| | TGHFYINGYRYFSLGNVEAVNFNVTNAATTVCTVALASYADVLVNVSQ | |
| | TAIANIIYCNSVINRLRCDQLSFDVPDGFYSTSPIQPVELPVSIVSLPVYHK | |
| | HTFIVLYVNFEHQRGPGKCYNCRPAVINITLANFNETKGPLCVDTSHFTT | |
| | QFVDNVKLARWSASIDTGNCPFSFGKVINFVKFGSVCFSLKYIPGGCAM | |
| | TKYNIYDVSGVGVIRISNDTFLNGITYTSTSGNLLGFKDVTNGTIYSITPC | |
| | PIMANLVNYKSHNIGSLYVSWSDGDVITGVPKPVEGVSSFMNVTLNKC | |
| | TKYNIYDVSGVGVIRISNDTFLNGITYTSTSGNLLGFKDVTNGTIYSITPC | |
| | NPPDQLVVYQQAVVGAMLSENFTSYGFSNVVEMPKFFYASNGTYNCT | |
| | DAVLTYSSFGVCADGSIIAVQPRNVSYDSVSAIVTANLSIPSNWTTSVQV | |
| | EYLQITSTPIVVDCSTYVCNGNVRCVELLKQYTSACKTIEDALRNSAML | |
| | ESADVSEMLTFDKKAFTLANVSSFGDYNLSSVIPSLPRSGSRVAGRSAIE | |
| | DILFSKLVTSGLGTVDADYKKCTKGLSIADLACAQYYNGIMVLPGAAD | |
| | AERMAMYTGSLIGGIALGGLTSAASIPFSLAIQSRLNYVALQTDVLQEN | |
| | QRILAASFNKAMTNIVDAFTGVNDAITQTSQALQTVATALNKIQDVVN | |
| | QQGNSLNHLTSQLRQNFQAISSSIQAIYDRLDPPQADQQVDRLITGRLAA | |
| | LNVFVSHTLTKYTEVRASRQLAQQKVNECVKSQSKRYGFCGNGTHIFS | |
| | LVNAAPEGLVFLHTVLLPTQYKDVEAWSGLCVDGINGYVLRQPNLALY | |
| | KEGNYYRITSRIMFEPRIPTIADFVQIENCNVTFVNISRSELQTIVPEYIDV | |
| | NKTLQELSYKLPNYTVPDLVVEQYNQTILNLTSEISTLENKSAELNYTVQ | |
| | KLQTLIDNINSTLVDLKWLNRVETYIKWPWWVWLCISVVLIFVVSMLLL | |
| | CCCSTGCCGFFSCFASSIRGCCESTKLPYYDVEKIHIQ | |
| PolyA tail | 100 nt | |

NL63_S2P

| SEQ ID NO: 12 consists of from 5′ end to 3′ end: 5′ UTR | | 12 |
|---|---|---|
| SEQ ID NO: 2, mRNA ORF SEQ ID NO: 13, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGAAGCUGUUCCUGAUCUUACUGAUCCUGCCCCUGGUGAGCUGC | 13 |
| Construct | UUCUCUACCUGCAACAGCAACGCCAGCAUCAGCAUGCUCCAACUG | |
| (excluding the | GGCGUGCCUGACAACAGCUCUACCAUCGUGACCGGCCUGCUGCCC | |
| stop codon) | GUGCACUGGAUCUGCGCCAACCAAAGCACCAGCACCUACCCCGCAA | |
| | ACGGCUUCUUCUACAUCGACGUGGGCAAGCACCGGAGCGCCUUCG | |
| | CCCUGCACAGCGGCUACUACGACGCCAACCAGUACUAUAUCUACC | |
| | UGACCAACAACAUCAGCCUGAACGCCCCAGUGACCCUGAAGAUCU | |
| | GCAAGUUCGGCAACACCAGCUUCGACUUCCUGAGCAACGUGAGUA | |
| | CCAGCCACCACUGCAUUGUGAACUCAAGCUUCACCGAGCAGCUGG | |
| | GAGUGCCCCUGGGCAUCACCAUCAGCGGCGAGACCGUGCGGCUGC | |
| | ACCUGUACAACGCCACCCGGACCUUCUACGUGCCCGCCGCCUACAA | |
| | GCUGACCAAGCUGAGCGUGAAGUGCUACUUCAGCGAGAGCUGCGU | |
| | GUUUAGCGUGGUAAACGCCACCGUGACCGUGAACGUGACCACCCU | |
| | GAACGGCCGGAUCGUGAACUACACCGUGUGCGACGACUGCAACGG | |
| | CUACACCGACAACAUCUUUAGCGUGCAGCAGGACGGGCGGAUCCC | |
| | CAACGGCUUUAGCUUCAACAACUGGUUCCUGCUGACCAACGGCAG | |
| | CACCCUGGUGGACGGCGUGAGCCGGCUGUACCAGCCCCUGCGGCU | |
| | GACCUGCCUGUGGCCCGUGCCCGGUCUGAAGAGCUCCACCGGCUU | |
| | CGUGUACUUCAACGCCACUGGCAGCGACGUGAAUUGUAACGGCUA | |
| | UCAGCACCACAGCGUGGCCGACGUGAUGCGGUACAACCUGAACUU | |
| | CAGCUCAAACAGCGUGGACAACCUUAAGAGCGGCGUGAUCGUGUU | |
| | UAAGACACUGCAGUACGACGUGCUCUUCUACUGCAGCAACAGCAG | |
| | CAGCGGCGUGCUGGACACCACCAUACCCUUCGGCCCCAGCAGCCAG | |
| | CCCUACUAUUGCUUCAUCAACAGCACCAUCAACACCACCCACGUGU | |
| | CUACCUUCGUGGGCGUGCUGCCGCCCAUCGUGCGGGAGAUCGUUG | |
| | UGGCCCGGACCGGCCAGUUCUACAUCAACGGCUUCAAGUACUUCG | |
| | ACCUUGGCUUCAUCGAGGCCGUGAACUUCAACGUGACAACCGCCA | |
| | GCGCCACCGACUUCUGGACCGUGGCCUUCGCCACCUUUGUGGACG | |
| | UGCUGGUCAACGUGAGCGCCACCAACAUCCAGAAUCUGCUGUACU | |
| | GCGACAGCCCCUUCGAGAAGCUGCAGUGCGAGCACCUGCAGUUCG | |
| | GCCUGCAGGACGGCUUCUACAGCGCCAACUUCCUGGACGACAACG | |
| | UGCUGCCCGACACCUACGUCGCCCUGCCCAUCUACUACCAGCACAC | |
| | CGACAUCAACUUCACCGCCACCGCCAGCUUCGGAGGCAGCUGUUA | |
| | CGUGUGCAAGCCCCACCAGGUGAACAUCAGUCUGAACGGCAACAC | |
| | UAGUGUGUGCGUGCGGACCAGCCACUUCAGCAUACGGUACAUCUA | |
| | CAACCGGGUGAAGAGCGGAAGCCCCGGCGACAGCAGCUGGCACAU | |
| | CUACCUAAAGAGCGGCACCUGCCCCUUCAGCUUUAGCAAGCUGAA | |
| | CAACUUCCAGAAGUUCAAGACCAUUUGCUUCAGCACCGUGGCAGU | |
| | GCCCGGCAGCUGUAACUUUCCCCUGGAAGCCACCUGGCACUACACC | |
| | AGCUACACCAUCGUGGGCGCCCUGUACGUGACCUGGAGCGAGGGC | |
| | AACAGCAUCACCGGCGUGCCCUACCCCGUGAGCGGCAUCCGGGAG | |
| | UUCAGCAACCUGGUGCUGAAUAACUGCACGAAGUACAACAUCUAC | |
| | GAUUACGUGGGCACCGGCAUCAUCCGGUCUAGCAACCAGAGCCUG | |
| | GCCGGCGGCAUCACCUACGUAAGCAACAGCGGCAACCUGCUGGGC | |
| | UUCAAGAACGUUAGCACCGGCAACAUCUUCAUCGUGACGCCCUGC | |
| | AACCAGCCCGACCAGGUGGCCGUGUACCAGCAGAGCAUCAUCGGC | |
| | GCCAUGACCGCCGUGAACGAGAGCCGGUACGGUCUGCAGAACCUG | |
| | CUGCAGCUGCCCAACUUCUACUACGUGAGCAACGGCGGCAACAAC | |
| | UGCACCACCGCUGUGAUGACCUACAGCAACUUCGGCAUCUGCGCC | |
| | GACGGCAGCCUGAUCCCCGUGCGGCCCCGGAACAGCAGCGAUAAC | |
| | GGCAUCAGCGCCAUCAUCACCGCCAAUCUGAGCAUCCCCAGCAACU | |
| | GGACCACCAGCGUGCAGGUGGAGUACCUGCAGAUCACCUCAACUC | |
| | CCAUCGUGGUGGACUGCGCCACCUACGUGUGUAACGGCAAUCCCC | |
| | GGUGCAAGAACCUGUUGAAGCAGUACACCAGCGCCUGCAAGACCA | |
| | UCGAGGACGCCCUGAGGCUGAGCGCCCACCUGGAGACCAACGACG | |
| | UGAGCAGCAUGCUUACCUUCGACUCCAACGCCUUCAGCCUGGCCA | |
| | ACGUGACUAGCUUCGGCGACUAUAACCUGAGCUCUGUGCUGCCCC | |
| | AGCGGAACAUCCGGAGCUCCCGGAUCGCCGGCAGAAGCGCCCUGG | |
| | AGGACCUGCUGUUCAGCAAGGUGGUGACCAGCGGCCUGGGCACCG | |
| | UGGACGUGGAUUACAAGAGCUGCACCAAGGGCCUGAGCAUCGCCG | |
| | AUCUGGCCUGCGCCCAGUACUACAACGGCAUUAUGGUGUUACCCG | |
| | GCGUUGCCGACGCCGAGCGGAUGGCCAUGUACACCGGCAGUCUGA | |
| | UCGGCGGCAUGGUGCUGGGCGGCCUGACAAGCGCCGCCGCCAUCC | |
| | CCUUUAGCCUGGCUCUGCAGGCCCGGCUGAACUACGUGGCCCUGC | |
| | AAACCGACGUGCUGCAGGAGAACCAGAAGAUCCUGGCCGCCUCAU | |
| | UCAACAAGGCCAUCAACAACAUUGUGGCCAGCUUCAGCAGCGUGA | |
| | ACGACGCCAUCACCCAGACCGCCGAGGCCAUCCACACCGUCACCAU | |
| | CGCCCUGAACAAGAUCCAGGACGUGGUGAACCAGCAGGGCUCCGC | |
| | CCUUAACCACCUGACCAGCCAGCUGCGGCACAACUUUCAGGCCAUC | |
| | AGCAAUAGCAUCCAGGCCAUCUACGACCGGCUGGACCCACCCCAG | |
| | GCCGACCAGCAGGUGGACCGGCUGAUCACCGGCCGGUUGGCCGCA | |
| | CUGAACGCCUUCGUGAGCCAGGUGCUGAACAAGUACACCGAGGUG | |
| | CGGAGCUCUCGGCGGCUGGCCCAGCAGAAGAUCAACGAGUGCGUG | |
| | AAGAGCCAGAGCAACCGGUACGGCUUCUGCGGCAACGGCACCCAC | |
| | AUCUUCAGCAUCGUGAACAGCGCCCCUGACGGCCUACUGUUCCUG | |
| | CACACCGUGCUGCUGCCCACCGACUACAAGAACGUGAAGGCCUGG | |
| | AGCGGCAUUUGCGUGGACGGCAUCUACGGCUACGUGCUGCGGCAG | |
| | CCCAACUUGGUGCUGUACAGCGACAACGGCGUGUUCCGGAUCACC | |
| | AGCCGGGUGAUGUUCCAGCCCCGACUGCCCGUUCUGAGCGACUUU | |
| | GUGCAGAUCUACAACUGCAACGUGACCUUCGUGAACAUUAGCCGG | |
| | GUUAAGCUGCAUACCGUGAUCCCCGACUACGUGGACGUUAACAAG | |
| | ACCCUGCAGGAGUUCGCCCAGAACUUGCCCAAGUACGUGAAGCCC | |
| | AAUUUCGACCUGACACCCUUCAACCUGACCUACCUGAACCUGAGC | |
| | AGCGAGCUGAAGCAGCUGGAGGCCAAGACCGCCAGCCUGUUCCAG | |
| | ACCACCGUGGAGCUGCAGGGCCUGAUCGACCAGAUCAAUAGCACA | |
| | UACGUGGACCUGAAGCUGCUGAACCGGUUCGAGAACUACAUCAAG | |
| | UGGCCCUGGUGGGUGUGGCUGAUCAUCAGCGUGGUGUUCGUGGUG | |
| | CUGCUGAGCCUGCUGGUGUUCUGCUGCCUGAGCACCGGCUGCUGU | |
| | GGCUGCUGCAACUGCCUGACCUCCAGCAUGCGGGGCUGCUGCGAC | |
| | UGCGGCUCCACCAAGCUGCCCUACUACGAGUUCGAGAAGGUGCAC | |
| | GUGCAG | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MKLFLILLILPLVSCFSTCNSNASISMLQLGVPDNSSTIVTGLLPVHWICA | 14 |
| amino acid | NQSTSTYPANGFFYIDVGKHRSAFALHSGYYDANQYYIYLTNNISLNAP | |
| sequence | VTLKICKFGNTSFDFLSNVSTSHHCIVNSSFTEQLGVPLGITISGETVRLH | |
| | LYNATRTFYVPAAYKLTKLSVKCYFSESCVFSVVNATVTVNVTTLNGRI | |
| | VNYTVCDDCNGYTDNIFSVQQDGRIPNGFSFNNWFLLTNGSTLVDGVS | |
| | RLYQPLRLTCLWPVPGLKSSTGFVYFNATGSDVNCNGYQHHSVADVM | |
| | RYNLNFSSNSVDNLKSGVIVFKTLQYDVLFYCSNSSSGVLDTTIPFGPSS | |
| | QPYYCFINSTINTTHVSTFVGVLPPIVREIVVARTGQFYINGFKYFDLGFI | |
| | EAVNFNVTTASATDFWTVAFATFVDVLVNVSATNIQNLLYCDSPFEKL | |
| | QCEHLQFGLQDGFYSANFLDDNVLPDTYVALPIYYQHTDINFTATASFG | |
| | GSCYVCKPHQVNISLNGNTSVCVRTSHFSIRYIYNRVKSGSPGDSSWHIY | |
| | LKSGTCPFSFSKLNNFQKFKTICFSTVAVPGSCNFPLEATWHYTSYTIVG | |
| | ALYVTWSEGNSITGVPYPVSGIREFSNLVLNNCTKYNIYDYVGTGIIRSS | |
| | NQSLAGGITYVSNSGNLLGFKNVSTGNIFIVTPCNQPDQVAVYQQSIIGA | |
| | MTAVNESRYGLQNLLQLPNFYYVSNGGNNCTTAVMTYSNFGICADGSL | |
| | IPVRPRNSSDNGISAIITANLSIPSNWTTSVQVEYLQITSTPIVVDCATYVC | |
| | NGNPRCKNLLKQYTSACKTIEDALRLSAHLETNDVSSMLTFDSNAFSLA | |
| | NVTSFGDYNLSSVLPQRNIRSSRIAGRSALEDLLFSKVVTSGLGTVDVDY | |
| | KSCTKGLSIADLACAQYYNGIMVLPGVADAERMAMYTGSLIGGMVLG | |
| | GLTSAAAIPFSLALQARLNYVALQTDVLQENQKILAASENKAINNIVASF | |
| | SSVNDAITQTAEAIHTVTIALNKIQDVVNQQGSALNHLTSQLRHNFQAIS | |
| | NSIQAIYDRLDPPQADQQVDRLITGRLAALNAFVSQVLNKYTEVRSSRR | |
| | LAQQKINECVKSQSNRYGFCGNGTHIFSIVNSAPDGLLFLHTVLLPTDYK | |
| | NVKAWSGICVDGIYGYVLRQPNLVLYSDNGVFRITSRVMFQPRLPVLSD | |
| | FVQIYNCNVTFVNISRVKLHTVIPDYVDVNKTLQEFAQNLPKYVKPNFD | |
| | LTPFNLTYLNLSSELKQLEAKTASLFQTTVELQGLIDQINSTYVDLKLLN | |
| | RFENYIKWPWWVWLIISVVFVVLLSLLVFCCLSTGCCGCCNCLTSSMRG | |
| | CCDCGSTKLPYYEFEKVHVQ | |
| PolyA tail | 100 nt | |

OC43_S2P_Prko_AY585228

| SEQ ID NO: 15 consists of from 5′ end to 3′ end: 5′ UTR | | 15 |
|---|---|---|
| SEQ ID NO: 2, mRNA ORF SEQ ID NO: 16, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGUUCCUUAUCCUGCUGAUCAGCCUGCCCACCGCCUUCGCCGUG | 16 |
| Construct | AUCGGCGACCUGAAGUGCACCAGCGACAACAUCAAUGACAAGGAU | |
| (excluding the | ACCGGACCGCCUCCCAUCAGCACCGACACCGUGGACGUGACCAACG | |
| stop codon) | GCCUGGGCACCUACUACGUGCUGGACCGGGUGUACCUGAAUACCA | |
| | CCCUGUUCCUGAACGGCUAUUACCCCACCAGCGGCAGCACCUACCG | |
| | GAAUAUGGCGCUGAAGGGCAGCGUGCUGCUGAGCAGACUGUGGUU | |
| | CAAACCACCCUUCCUGAGCGACUUUAUCAACGGCAUUUUCGCGAA | |
| | GGUGAAGAACACCAAGGUGAUCAAGGACAGAGUGAUGUACAGCGA | |
| | GUUCCCCGCCAUCACCAUUGGGAGCACCUUCGUCAACACCAGCUAC | |
| | UCUGUGGUGGUGCAGCCCCGGACCAUCAACAGCACCCAGGACGGU | |
| | GACAACAAGCUGCAGGGCCUGCUGGAGGUGAGCGUGUGCCAGUAC | |
| | AACAUGUGCGAGUAUCCCCAGACCAUCUGCCACCCCAACCUGGGC | |
| | AACCACAGAAAGGAGCUGUGGCACCUGGACACCGGAGUGGUGAGC | |
| | UGCCUGUACAAGCGGAACUUCACCUACGACGUGAACGCUGACUAC | |
| | CUGUACUUCCACUUCUACCAGGAGGGCGGCACCUUCUACGCCUAC | |
| | UUCACCGAUACCGGCGUGGUGACCAAGUUCCUGUUCAACGUGUAC | |
| | CUGGGCAUGGCCCUGAGCCACUACUACGUUAUGCCCCUGACCUGU | |
| | AACAGCAAGCUGACCCUGGAGUACUGGGUGACCCCUCUGACCAGC | |
| | CGGCAGUACCUGCUGGCCUUCAACCAGGACGGCAUCAUCUUCAAC | |
| | GCCGUGGACUGCAUGUCAGACUUCAUGAGCGAGAUCAAGUGCAAG | |
| | ACCCAGAGCAUCGCCCCUCCCACCGGCGUGUACGAGCUGAACGGU | |
| | UACACCGUGCAGCCCAUCGCCGACGUGUACCGGCGGAAGCCCAACC | |
| | UGCCCAACUGCAACAUCGAGGCCUGGCUGAACGACAAGAGCGUGC | |
| | CCAGCCCACUGAACUGGGAGCGGAAGACCUUCAGUAACUGCAACU | |
| | UCAACAUGAGCAGCCUGAUGAGCUUCAUCCAGGCCGACUCAUUCA | |
| | CCUGCAACAACAUCGACGCCGCCAAGAUCUACGGCAUGUGCUUCA | |
| | GCAGCAUCACCAUCGACAAGUUCGCCAUCCCAAACGGCCGGAAGG | |
| | UGGACUUACAGCUGGGCAAUCUGGGCUACCUGCAGAGCUUCAACU | |
| | ACCGGAUCGACACGACCGCCACCUCCUGCCAGUUAUACUACAAUC | |
| | UGCCUGCCGCCAACGUGAGCGUGAGCCGCUUCAACCCCAGCACCUG | |
| | GAACAAGCGGUUCGGCUUCAUCGAGGACAGCGUGUUCAAACCCAG | |
| | ACCCGCCGGCGUGCUGACAAACCACGACGUGGUGUACGCCCAGCA | |
| | CUGCUUUAAGGCCCCUAAGAACUUCUGCCCCUGCAAGCUGAAUGG | |
| | CAGCUGCGUCGGGUCUGGCCCCGGAAAGAACAACGGGAUUGGCAC | |
| | CUGCCCCGCUGGCACCAACUACCUGACCUGCGACAACCUCUGCACC | |
| | CCUGACCCCAUCACCUUCACCGGCACCUACAAGUGUCCUCAGACCA | |
| | AGAGCCUGGUGGGCAUCGGCGAGCACUGCAGCGGCCUGGCAGUGA | |
| | AGAGCGACUACUGCGGCGGCAACAGCUGCACCUGCAGACCCCAGG | |
| | CCUUCCUGGGCUGGAGCGCCGACAGCUGCCUGCAGGGCGACAAGU | |
| | GCAACAUCUUCGCCAAUUUCAUCCUGCACGACGUCAACAGCGGAC | |
| | UGACCUGCAGCACCGACCUGCAGAAGGCCAACACCGACAUCAUCC | |
| | UGGGCGUGUGCGUGAACUACGACCUGUACGGCAUCCUGGGCCAGG | |
| | GCAUCUUCGUGGAGGUCAACGCCACCUAUUACAACAGCUGGCAGA | |
| | ACCUGCUGUACGACAGCAACGGUAAUUUAUACGGCUUCCGGGACU | |
| | ACAUCACCAACCGGACCUUCAUGAUCCGGAGCUGCUACAGCGGCC | |
| | GGGUGAGCGCAGCUUUCCACGCCAACAGCAGCGAGCCCGCCCUGC | |
| | UGUUUCGGAACAUCAAGUGUAACUACGUGUUCAACAACAGCCUGA | |
| | CCCGGCAGCUGCAGCCUAUCAACUACUUCGACAGCUACCUGGGCU | |
| | GCGUGGUGAACGCAUACAACAGCACUGCCAUCAGCGUGCAGACCU | |
| | GCGACCUGACCGUGGGCUCCGGGUACUGCGUGGACUACAGCAAGA | |
| | ACGGCGGCUCCGGCGGAGCUAUCACCACCGGCUACCGGUUCACCA | |
| | ACUUCGAGCCCUUCACCGUGAACAGCGUGAACGACAGCCUGGAGC | |
| | CCGUGGGCGGACUGUACGAGAUCCAGAUCCCCAGCGAGUUCACCA | |
| | UCGGCAACAUGGUGGAGUUCAUCCAGACCUCAAGCCCCAAGGUGA | |
| | CCAUCGACUGCGCCGCCUUCGUGUGCGGCGACUACGCCGCCUGCAA | |
| | GAGCCAGCUGGUGGAGUACGGCAGCUUCUGCGACAAUAUCAACGC | |
| | CAUCCUGACCGAGGUGAACGAACUGCUGGACACCACCCAGUUACA | |
| | GGUGGCCAACAGUCUGAUGAACGGCGUGACCCUGAGUACCAAGCU | |
| | GAAGGACGGAGUGAACUUCAACGUGGACGACAUCAAUUUCAGCCC | |
| | CGUGCUGGGCUGCCUGGGCAGCGAGUGCAGCAAGGCCAGUAGCCG | |
| | GAGCGCCAUAGAGGACCUGCUGUUCGACAAGGUGAAACUGAGCGA | |
| | CGUGGGCUUCGUGGAGGCCUACAACAACUGCACCGGUGGCGCCGA | |
| | GAUCCGGGACCUGAUCUGCGUGCAGAGCUACAAGGGCAUCAAAGU | |
| | GCUGCCACCUCUGCUGAGCGAGAACCAGAUCAGCGGCUACACCCU | |
| | CGCCGCCACCAGCGCUAGCCUGUUUCCUCCUUGGACUGCUGCCGCA | |
| | GGCGUGCCCUUCUACCUGAACGUGCAGUACCGGAUUAACGGCCUC | |
| | GGUGUGACCAUGGACGUGCUGAGCCAGAACCAGAAGCUGAUCGCC | |
| | AACGCCUUCAACAACGCCCUGUACGCCAUCCAGGAGGGAUUCGAC | |
| | GCCACCAACAGCGCCCUGGUGAAGAUCCAGGCGGUAGUGAACGCC | |
| | AACGCUGAGGCCCUGAACAACCUGCUGCAACAGCUGAGCAACCGG | |
| | UUCGGCGCCAUCAGCGCCAGCCUGCAGGAGAUCCUGAGCCGGCUG | |
| | GAUCCACCAGAGGCCGAGGCCCAGAUCGACCGGCUGAUCAACGGC | |
| | CGGCUGACCGCCCUGAACGCCUACGUGAGCCAGCAGCUGAGCGAC | |
| | AGCACCCUGGUCAAGUUCAGCGCCGCCCAGGCCAUGGAGAAAGUG | |
| | AACGAGUGCGUGAAGAGCCAGAGCAGCCGGAUCAACUUCUGCGGC | |
| | AACGGCAACCACAUCAUCAGCCUGGUGCAGAACGCCCCUUACGGC | |
| | CUGUACUUUAUCCACUUCAGCUACGUGCCCACCAAGUACGUGACC | |
| | GCCAGAGUGAGCCCCGGCCUGUGCAUCGCCGGCGACCGGGGAAUC | |
| | GCUCCCAAGAGCGGCUACUUCGUGAACGUGAACAACACCUGGAUG | |
| | UACACCGGCAGCGGAUACUACUACCCCGAGCCCAUCACCGAGAAC | |
| | AACGUGGUGGUGAUGAGCACCUGCGCAGUGAACUACACCAAGGCU | |
| | CCUUACGUGAUGCUGAACACCUCUAUCCCAAAUCUGCCCGACUUC | |
| | AAGGAGGAGCUGGACCAGUGGUUCAAGAACCAGACCAGCGUGGCC | |
| | CCUGAUCUGAGCCUGGACUACAUUAACGUGACCUUCCUGGACCUG | |
| | CAGGUGGAGAUGAACCGGCUGCAGGAGGCCAUCAAGGUGCUGAAC | |
| | CAGUCAUACAUCAACCUGAAGGACAUCGGCACCUACGAGUACUAC | |
| | GUGAAGUGGCCCUGGUACGUGUGGCUGCUGAUCUGCCUGGCCGGC | |
| | GUGGCCAUGCUGGUGCUGCUGUUCUUCAUCUGCUGCUGCACCGGC | |
| | UGCGGCACCAGCUGCUUCAAGAAGUGCGGCGGCUGCUGCGACGAC | |
| | UACACCGGAUACCAGGAGCUGGUGAUCAAGACCAGCCACGACGAC | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MFLILLISLPTAFAVIGDLKCTSDNINDKDTGPPPISTDTVDVTNGLGTYY | 17 |
| amino acid | VLDRVYLNTTLFLNGYYPTSGSTYRNMALKGSVLLSRLWFKPPFLSDFI | |
| sequence | NGIFAKVKNTKVIKDRVMYSEFPAITIGSTFVNTSYSVVVQPRTINSTQD | |
| | GDNKLQGLLEVSVCQYNMCEYPQTICHPNLGNHRKELWHLDTGVVSC | |
| | LYKRNFTYDVNADYLYFHFYQEGGTFYAYFTDTGVVTKFLFNVYLGM | |
| | ALSHYYVMPLTCNSKLTLEYWVTPLTSRQYLLAFNQDGIIFNAVDCMS | |
| | DFMSEIKCKTQSIAPPTGVYELNGYTVQPIADVYRRKPNLPNCNIEAWL | |
| | NDKSVPSPLNWERKTFSNCNFNMSSLMSFIQADSFTCNNIDAAKIYGMC | |
| | FSSITIDKFAIPNGRKVDLQLGNLGYLQSFNYRIDTTATSCQLYYNLPAA | |
| | NVSVSRFNPSTWNKRFGFIEDSVFKPRPAGVLTNHDVVYAQHCFKAPK | |
| | NFCPCKLNGSCVGSGPGKNNGIGTCPAGTNYLTCDNLCTPDPITFTGTY | |
| | KCPQTKSLVGIGEHCSGLAVKSDYCGGNSCTCRPQAFLGWSADSCLQG | |
| | DKCNIFANFILHDVNSGLTCSTDLQKANTDIILGVCVNYDLYGILGQGIF | |
| | VEVNATYYNSWQNLLYDSNGNLYGFRDYITNRTFMIRSCYSGRVSAAF | |
| | HANSSEPALLFRNIKCNYVFNNSLTRQLQPINYFDSYLGCVVNAYNSTAI | |
| | SVQTCDLTVGSGYCVDYSKNGGSGGAITTGYRFTNFEPFTVNSVNDSLE | |
| | PVGGLYEIQIPSEFTIGNMVEFIQTSSPKVTIDCAAFVCGDYAACKSQLV | |
| | EYGSFCDNINAILTEVNELLDTTQLQVANSLMNGVTLSTKLKDGVNFNV | |
| | DDINFSPVLGCLGSECSKASSRSAIEDLLFDKVKLSDVGFVEAYNNCTG | |
| | GAEIRDLICVQSYKGIKVLPPLLSENQISGYTLAATSASLFPPWTAAAGV | |
| | PFYLNVQYRINGLGVTMDVLSQNQKLIANAFNNALYAIQEGFDATNSA | |
| | LVKIQAVVNANAEALNNLLQQLSNRFGAISASLQEILSRLDPPEAEAQID | |
| | RLINGRLTALNAYVSQQLSDSTLVKFSAAQAMEKVNECVKSQSSRINFC | |
| | GNGNHIISLVQNAPYGLYFIHFSYVPTKYVTARVSPGLCIAGDRGIAPKS | |
| | GYFVNVNNTWMYTGSGYYYPEPITENNVVVMSTCAVNYTKAPYVML | |
| | NTSIPNLPDFKEELDQWFKNQTSVAPDLSLDYINVTFLDLQVEMNRLQE | |
| | AIKVLNQSYINLKDIGTYEYYVKWPWYVWLLICLAGVAMLVLLFFICC | |
| | CTGCGTSCFKKCGGCCDDYTGYQELVIKTSHDD | |
| PolyA tail | 100 nt | |

HKU1_S2P_Prko_AY585228

| SEQ ID NO: 18 consists of from 5′ end to 3′ end: 5′ UTR | | 18 |
|---|---|---|
| SEQ ID NO: 2, mRNA ORF SEQ ID NO: 19, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGUUCCUUAUCCUGCUGAUCAGCCUGCCCACCGCCUUCGCCGUG | 19 |
| Construct | AUCGGCGACCUGAAGUGCACCAGCGACAACAUCAAUGACAAGGAU | |
| (excluding the | ACCGGACCGCCUCCCAUCAGCACCGACACCGUGGACGUGACCAACG | |
| stop codon) | GCCUGGGCACCUACUACGUGCUGGACCGGGUGUACCUGAAUACCA | |
| | CCCUGUUCCUGAACGGCUAUUACCCCACCAGCGGCAGCACCUACCG | |
| | GAAUAUGGCGCUGAAGGGCAGCGUGCUGCUGAGCAGACUGUGGUU | |
| | CAAACCACCCUUCCUGAGCGACUUUAUCAACGGCAUUUUCGCGAA | |
| | GGUGAAGAACACCAAGGUGAUCAAGGACAGAGUGAUGUACAGCGA | |
| | GUUCCCCGCCAUCACCAUUGGGAGCACCUUCGUCAACACCAGCUAC | |
| | UCUGUGGUGGUGCAGCCCCGGACCAUCAACAGCACCCAGGACGGU | |
| | GACAACAAGCUGCAGGGCCUGCUGGAGGUGAGCGUGUGCCAGUAC | |
| | AACAUGUGCGAGUAUCCCCAGACCAUCUGCCACCCCAACCUGGGC | |
| | AACCACAGAAAGGAGCUGUGGCACCUGGACACCGGAGUGGUGAGC | |
| | UGCCUGUACAAGCGGAACUUCACCUACGACGUGAACGCUGACUAC | |
| | CUGUACUUCCACUUCUACCAGGAGGGCGGCACCUUCUACGCCUAC | |
| | UUCACCGAUACCGGCGUGGUGACCAAGUUCCUGUUCAACGUGUAC | |
| | CUGGGCAUGGCCCUGAGCCACUACUACGUUAUGCCCCUGACCUGU | |
| | AACAGCAAGCUGACCCUGGAGUACUGGGUGACCCCUCUGACCAGC | |
| | CGGCAGUACCUGCUGGCCUUCAACCAGGACGGCAUCAUCUUCAAC | |
| | GCCGUGGACUGCAUGUCAGACUUCAUGAGCGAGAUCAAGUGCAAG | |
| | ACCCAGAGCAUCGCCCCUCCCACCGGCGUGUACGAGCUGAACGGU | |
| | UACACCGUGCAGCCCAUCGCCGACGUGUACCGGCGGAAGCCCAACC | |
| | UGCCCAACUGCAACAUCGAGGCCUGGCUGAACGACAAGAGCGUGC | |
| | CCAGCCCACUGAACUGGGAGCGGAAGACCUUCAGUAACUGCAACU | |
| | UCAACAUGAGCAGCCUGAUGAGCUUCAUCCAGGCCGACUCAUUCA | |
| | CCUGCAACAACAUCGACGCCGCCAAGAUCUACGGCAUGUGCUUCA | |
| | GCAGCAUCACCAUCGACAAGUUCGCCAUCCCAAACGGCCGGAAGG | |
| | UGGACUUACAGCUGGGCAAUCUGGGCUACCUGCAGAGCUUCAACU | |
| | ACCGGAUCGACACGACCGCCACCUCCUGCCAGUUAUACUACAAUC | |
| | UGCCUGCCGCCAACGUGAGCGUGAGCCGCUUCAACCCCAGCACCUG | |
| | GAACAAGCGGUUCGGCUUCAUCGAGGACAGCGUGUUCAAACCCAG | |
| | ACCCGCCGGCGUGCUGACAAACCACGACGUGGUGUACGCCCAGCA | |
| | CUGCUUUAAGGCCCCUAAGAACUUCUGCCCCUGCAAGCUGAAUGG | |
| | CAGCUGCGUCGGGUCUGGCCCCGGAAAGAACAACGGGAUUGGCAC | |
| | CUGCCCCGCUGGCACCAACUACCUGACCUGCGACAACCUCUGCACC | |
| | CCUGACCCCAUCACCUUCACCGGCACCUACAAGUGUCCUCAGACCA | |
| | AGAGCCUGGUGGGCAUCGGCGAGCACUGCAGCGGCCUGGCAGUGA | |
| | AGAGCGACUACUGCGGCGGCAACAGCUGCACCUGCAGACCCCAGG | |
| | CCUUCCUGGGCUGGAGCGCCGACAGCUGCCUGCAGGGCGACAAGU | |
| | GCAACAUCUUCGCCAAUUUCAUCCUGCACGACGUCAACAGCGGAC | |
| | UGACCUGCAGCACCGACCUGCAGAAGGCCAACACCGACAUCAUCC | |
| | UGGGCGUGUGCGUGAACUACGACCUGUACGGCAUCCUGGGCCAGG | |
| | GCAUCUUCGUGGAGGUCAACGCCACCUAUUACAACAGCUGGCAGA | |
| | ACCUGCUGUACGACAGCAACGGUAAUUUAUACGGCUUCCGGGACU | |
| | ACAUCACCAACCGGACCUUCAUGAUCCGGAGCUGCUACAGCGGCC | |
| | GGGUGAGCGCAGCUUUCCACGCCAACAGCAGCGAGCCCGCCCUGC | |
| | UGUUUCGGAACAUCAAGUGUAACUACGUGUUCAACAACAGCCUGA | |
| | CCCGGCAGCUGCAGCCUAUCAACUACUUCGACAGCUACCUGGGCU | |
| | GCGUGGUGAACGCAUACAACAGCACUGCCAUCAGCGUGCAGACCU | |
| | GCGACCUGACCGUGGGCUCCGGGUACUGCGUGGACUACAGCAAGA | |
| | ACGGCGGCUCCGGCGGAGCUAUCACCACCGGCUACCGGUUCACCA | |
| | ACUUCGAGCCCUUCACCGUGAACAGCGUGAACGACAGCCUGGAGC | |
| | CCGUGGGCGGACUGUACGAGAUCCAGAUCCCCAGCGAGUUCACCA | |
| | UCGGCAACAUGGUGGAGUUCAUCCAGACCUCAAGCCCCAAGGUGA | |
| | CCAUCGACUGCGCCGCCUUCGUGUGCGGCGACUACGCCGCCUGCAA | |
| | GAGCCAGCUGGUGGAGUACGGCAGCUUCUGCGACAAUAUCAACGC | |
| | CAUCCUGACCGAGGUGAACGAACUGCUGGACACCACCCAGUUACA | |
| | GGUGGCCAACAGUCUGAUGAACGGCGUGACCCUGAGUACCAAGCU | |
| | GAAGGACGGAGUGAACUUCAACGUGGACGACAUCAAUUUCAGCCC | |
| | CGUGCUGGGCUGCCUGGGCAGCGAGUGCAGCAAGGCCAGUAGCCG | |
| | GAGCGCCAUAGAGGACCUGCUGUUCGACAAGGUGAAACUGAGCGA | |
| | CGUGGGCUUCGUGGAGGCCUACAACAACUGCACCGGUGGCGCCGA | |
| | GAUCCGGGACCUGAUCUGCGUGCAGAGCUACAAGGGCAUCAAAGU | |
| | GCUGCCACCUCUGCUGAGCGAGAACCAGAUCAGCGGCUACACCCU | |
| | CGCCGCCACCAGCGCUAGCCUGUUUCCUCCUUGGACUGCUGCCGCA | |
| | GGCGUGCCCUUCUACCUGAACGUGCAGUACCGGAUUAACGGCCUC | |
| | GGUGUGACCAUGGACGUGCUGAGCCAGAACCAGAAGCUGAUCGCC | |
| | AACGCCUUCAACAACGCCCUGUACGCCAUCCAGGAGGGAUUCGAC | |
| | GCCACCAACAGCGCCCUGGUGAAGAUCCAGGCGGUAGUGAACGCC | |
| | AACGCUGAGGCCCUGAACAACCUGCUGCAACAGCUGAGCAACCGG | |
| | UUCGGCGCCAUCAGCGCCAGCCUGCAGGAGAUCCUGAGCCGGCUG | |
| | GAUCCACCAGAGGCCGAGGCCCAGAUCGACCGGCUGAUCAACGGC | |
| | CGGCUGACCGCCCUGAACGCCUACGUGAGCCAGCAGCUGAGCGAC | |
| | AGCACCCUGGUCAAGUUCAGCGCCGCCCAGGCCAUGGAGAAAGUG | |
| | AACGAGUGCGUGAAGAGCCAGAGCAGCCGGAUCAACUUCUGCGGC | |
| | AACGGCAACCACAUCAUCAGCCUGGUGCAGAACGCCCCUUACGGC | |
| | CUGUACUUUAUCCACUUCAGCUACGUGCCCACCAAGUACGUGACC | |
| | GCCAGAGUGAGCCCCGGCCUGUGCAUCGCCGGCGACCGGGGAAUC | |
| | GCUCCCAAGAGCGGCUACUUCGUGAACGUGAACAACACCUGGAUG | |
| | UACACCGGCAGCGGAUACUACUACCCCGAGCCCAUCACCGAGAAC | |
| | AACGUGGUGGUGAUGAGCACCUGCGCAGUGAACUACACCAAGGCU | |
| | CCUUACGUGAUGCUGAACACCUCUAUCCCAAAUCUGCCCGACUUC | |
| | AAGGAGGAGCUGGACCAGUGGUUCAAGAACCAGACCAGCGUGGCC | |
| | CCUGAUCUGAGCCUGGACUACAUUAACGUGACCUUCCUGGACCUG | |
| | CAGGUGGAGAUGAACCGGCUGCAGGAGGCCAUCAAGGUGCUGAAC | |
| | CAGUCAUACAUCAACCUGAAGGACAUCGGCACCUACGAGUACUAC | |
| | GUGAAGUGGCCCUGGUACGUGUGGCUGCUGAUCUGCCUGGCCGGC | |
| | GUGGCCAUGCUGGUGCUGCUGUUCUUCAUCUGCUGCUGCACCGGC | |
| | UGCGGCACCAGCUGCUUCAAGAAGUGCGGCGGCUGCUGCGACGAC | |
| | UACACCGGAUACCAGGAGCUGGUGAUCAAGACCAGCCACGACGAC | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MFLILLISLPTAFAVIGDLKCTSDNINDKDTGPPPISTDTVDVTNGLGTYY | 20 |
| amino acid | VLDRVYLNTTLFLNGYYPTSGSTYRNMALKGSVLLSRLWFKPPFLSDFI | |
| sequence | NGIFAKVKNTKVIKDRVMYSEFPAITIGSTFVNTSYSVVVQPRTINSTQD | |
| | GDNKLQGLLEVSVCQYNMCEYPQTICHPNLGNHRKELWHLDTGVVSC | |
| | LYKRNFTYDVNADYLYFHFYQEGGTFYAYFTDTGVVTKFLFNVYLGM | |
| | ALSHYYVMPLTCNSKLTLEYWVTPLTSRQYLLAFNQDGIIFNAVDCMS | |
| | DFMSEIKCKTQSIAPPTGVYELNGYTVQPIADVYRRKPNLPNCNIEAWL | |
| | NDKSVPSPLNWERKTFSNCNFNMSSLMSFIQADSFTCNNIDAAKIYGMC | |
| | FSSITIDKFAIPNGRKVDLQLGNLGYLQSFNYRIDTTATSCQLYYNLPAA | |
| | NVSVSRFNPSTWNKRFGFIEDSVFKPRPAGVLTNHDVVYAQHCFKAPK | |
| | NFCPCKLNGSCVGSGPGKNNGIGTCPAGTNYLTCDNLCTPDPITFTGTY | |
| | KCPQTKSLVGIGEHCSGLAVKSDYCGGNSCTCRPQAFLGWSADSCLQG | |
| | DKCNIFANFILHDVNSGLTCSTDLQKANTDIILGVCVNYDLYGILGQGIF | |
| | VEVNATYYNSWQNLLYDSNGNLYGFRDYITNRTFMIRSCYSGRVSAAF | |
| | HANSSEPALLFRNIKCNYVENNSLTRQLQPINYFDSYLGCVVNAYNSTAI | |
| | SVQTCDLTVGSGYCVDYSKNGGSGGAITTGYRFTNFEPFTVNSVNDSLE | |
| | PVGGLYEIQIPSEFTIGNMVEFIQTSSPKVTIDCAAFVCGDYAACKSQLV | |
| | EYGSFCDNINAILTEVNELLDTTQLQVANSLMNGVTLSTKLKDGVNFNV | |
| | DDINFSPVLGCLGSECSKASSRSAIEDLLFDKVKLSDVGFVEAYNNCTG | |
| | GAEIRDLICVQSYKGIKVLPPLLSENQISGYTLAATSASLFPPWTAAAGV | |
| | PFYLNVQYRINGLGVTMDVLSQNQKLIANAFNNALYAIQEGFDATNSA | |
| | LVKIQAVVNANAEALNNLLQQLSNRFGAISASLQEILSRLDPPEAEAQID | |
| | RLINGRLTALNAYVSQQLSDSTLVKFSAAQAMEKVNECVKSQSSRINFC | |
| | GNGNHIISLVQNAPYGLYFIHFSYVPTKYVTARVSPGLCIAGDRGIAPKS | |
| | GYFVNVNNTWMYTGSGYYYPEPITENNVVVMSTCAVNYTKAPYVML | |
| | NTSIPNLPDFKEELDQWFKNQTSVAPDLSLDYINVTFLDLQVEMNRLQE | |
| | AIKVLNQSYINLKDIGTYEYYVKWPWYVWLLICLAGVAMLVLLFFICC | |
| | CTGCGTSCFKKCGGCCDDYTGYQELVIKTSHDD | |
| PolyA tail | 100 nt | |

229E_S2P_AF304460

| SEQ ID NO: 21 consists of from 5′ end to 3′ end: 5′ UTR | | 21 |
|---|---|---|
| SEQ ID NO: 2, mRNA ORF SEQ ID NO: 22, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGUUCGUGCUGCUGGUGGCCUACGCCCUGCUGCACAUCGCCGGC | 22 |
| Construct | UGCCAGACCACCAACGGCCUGAACACCAGCUACUCCGUUUGCAAC | |
| (excluding the | GGCUGCGUGGGCUACAGCGAGAACGUCUUCGCCGUGGAGAGCGGC | |
| stop codon) | GGCUACAUCCCUAGCGACUUCGCCUUCAACAACUGGUUCCUGCUG | |
| | ACCAACACCAGCUCCGUGGUGGACGGCGUGGUGCGGAGCUUCCAG | |
| | CCCCUGCUGCUGAACUGCCUGUGGAGUGUGAGCGGCCUGAGAUUC | |
| | ACCACCGGCUUCGUGUACUUCAACGGCACCGGCAGAGGCGACUGC | |
| | AAGGGCUUCAGCAGCGACGUGCUGUCUGACGUGAUUCGGUACAAC | |
| | CUGAACUUCGAGGAGAACCUGCGGCGGGGCACCAUCCUGUUCAAG | |
| | ACCAGCUACGGCGUGGUGGUGUUCUACUGCACCAACAACACCCUG | |
| | GUGAGCGGCGACGCCCACAUCCCCUUCGGCACCGUGCUGGGCAAC | |
| | UUCUACUGCUUCGUGAACACCACCAUCGGCAACGAGACGACCAGC | |
| | GCCUUCGUGGGCGCCCUGCCCAAGACCGUGCGGGAGUUCGUGAUC | |
| | AGCCGGACCGGCCACUUCUACAUUAACGGAUACCGGUACUUCACC | |
| | CUGGGCAACGUGGAGGCCGUGAACUUUAACGUGACCACCGCCGAG | |
| | ACCACCGACUUCUGCACCGUAGCCCUGGCCAGCUACGCCGACGUGC | |
| | UCGUAAACGUGAGCCAGACCAGCAUCGCCAACAUCAUCUACUGCA | |
| | ACAGCGUGAUCAAUCGGCUGCGGUGCGACCAGCUGAGCUUCGACG | |
| | UGCCCGACGGCUUCUACUCUACCAGCCCCAUCCAGAGCGUGGAAC | |
| | UGCCCGUGAGCAUCGUGAGCCUGCCCGUGUACCACAAGCACACCU | |
| | UCAUCGUGCUGUACGUGGACUUCAAGCCCCAGAGCGGCGGCGGCA | |
| | AGUGCUUCAACUGCUACCCCGCCGGCGUGAACAUCACCCUGGCUA | |
| | ACUUCAACGAGACCAAGGGCCCUCUGUGCGUUGAUACCAGCCAUU | |
| | UCACCACCAAGUACGUGGCCGUGUACGCCAACGUGGGCCGGUGGA | |
| | GCGCCAGCAUCAACACCGGCAACUGCCCCUUCAGCUUCGGCAAGG | |
| | UGAACAACUUCGUGAAGUUCGGCAGCGUGUGCUUCAGCCUGAAGG | |
| | ACAUCCCAGGCGGCUGCGCCAUGCCCAUCGUGGCCAACUGGGCCU | |
| | ACAGCAAGUACUACACCAUCGGCAGCUUAUACGUGAGCUGGAGCG | |
| | ACGGCGACGGCAUCACCGGCGUGCCCCAGCCCGUGGAGGGCGUGU | |
| | CUAGCUUCAUGAACGUGACCCUGGACAAGUGCACUAAGUACAACA | |
| | UCUACGACGUCAGCGGCGUGGGCGUGAUACGGGUGAGCAACGACA | |
| | CCUUCCUGAACGGCAUCACCUACACCAGCACCAGCGGCAACCUGCU | |
| | GGGCUUCAAGGACGUGACGAAGGGCACCAUCUACAGCAUCACCCC | |
| | UUGCAACCCUCCUGACCAGCUGGUGGUGUACCAGCAGGCCGUGGU | |
| | GGGCGCCAUGUUAAGCGAGAACUUCACCUCUUACGGCUUCAGCAA | |
| | CGUGGUGGAGCUGCCCAAGUUCUUCUACGCCAGCAACGGCACUUA | |
| | CAACUGCACCGACGCCGUGCUGACCUACUCAAGCUUCGGCGUGUG | |
| | CGCCGACGGCAGCAUCAUCGCCGUGCAGCCCCGGAACGUGAGCUA | |
| | CGACAGCGUGAGCGCCAUCGUGACCGCCAACCUGUCUAUCCCCAGC | |
| | AACUGGACCACCAGCGUGCAGGUGGAGUACCUGCAGAUUACCAGC | |
| | ACUCCCAUCGUGGUGGAUUGCAGCACCUACGUGUGCAACGGCAAC | |
| | GUGCGGUGCGUGGAGCUGCUGAAGCAGUACACCAGCGCUUGCAAG | |
| | ACCAUCGAGGACGCCCUGCGGAACAGCGCCAGACUGGAGUCUGCC | |
| | GACGUGAGCGAGAUGCUGACCUUCGACAAGAAGGCCUUCACACUG | |
| | GCCAACGUGAGCAGCUUUGGCGACUACAACCUGAGCAGCGUGAUU | |
| | CCAAGCCUUCCUACCAGCGGCUCUAGGGUGGCCGGCCGGUCUGCA | |
| | AUCGAGGACAUCUUGUUCAGCAAGCUGGUGACCAGCGGCCUGGGC | |
| | ACAGUGGACGCCGACUACAAGAAGUGCACCAAAGGCCUGAGCAUC | |
| | GCCGAUCUGGCCUGCGCCCAGUACUACAACGGAAUCAUGGUGCUU | |
| | CCAGGGGUGGCCGACGCAGAGCGGAUGGCCAUGUACACCGGCAGC | |
| | CUGAUCGGCGGAAUCGCCCUGGGCGGACUGACCAGCGCUGUGAGC | |
| | AUCCCCUUUAGCCUGGCCAUCCAGGCCCGGCUGAACUACGUGGCCC | |
| | UUCAGACUGACGUGCUGCAGGAGAACCAGAAGAUCCUGGCCGCCA | |
| | GCUUCAACAAGGCCAUGACCAACAUCGUGGACGCCUUCACAGGCG | |
| | UGAACGACGCCAUCACCCAGACCAGCCAGGCCCUGCAGACCGUGGC | |
| | CACCGCCCUCAACAAGAUCCAGGACGUGGUGAACCAGCAGGGCAA | |
| | CAGCCUGAACCACCUGACCUCUCAGCUGCGGCAGAACUUCCAGGCC | |
| | AUCAGCAGUAGCAUCCAAGCAAUCUACGACCGGCUGGAUCCACCA | |
| | CAGGCCGACCAGCAGGUGGACCGGCUGAUAACCGGCCGCCUGGCU | |
| | GCCCUUAACGUGUUCGUGAGCCAUACCCUGACCAAGUACACCGAG | |
| | GUGCGGGCCUCUCGGCAGCUGGCCCAGCAGAAGGUGAACGAGUGC | |
| | GUGAAGAGCCAGAGCAAGCGGUACGGCUUCUGCGGCAACGGGACC | |
| | CACAUCUUCAGCAUCGUGAACGCCGCCCCUGAAGGCCUGGUGUUC | |
| | CUGCACACCGUGCUACUGCCCACCCAGUACAAGGACGUUGAGGCC | |
| | UGGAGCGGCCUGUGCGUGGACGGGACCAACGGCUACGUGCUCCGG | |
| | CAGCCCAACCUGGCCCUGUACAAGGAGGGCAACUACUACCGGAUC | |
| | ACCAGCCGGAUCAUGUUCGAGCCCCGGAUCCCCACCAUGGCCGACU | |
| | UCGUGCAGAUCGAGAACUGCAACGUGACCUUUGUGAACAUUAGCC | |
| | GGAGCGAGCUGCAGACCAUCGUGCCCGAGUACAUCGACGUGAACA | |
| | AGACCCUGCAGGAGCUGAGCUACAAGCUGCCCAACUACACCGUUC | |
| | CCGACCUGGUGGUGGAGCAGUACAACCAGACCAUCCUGAACCUGA | |
| | CAAGCGAGAUAAGCACCCUGGAGAACAAGAGCGCCGAGCUGAAUU | |
| | ACACCGUGCAGAAGCUCCAGACACUGAUCGACAACAUCAACAGCA | |
| | CUCUGGUGGACCUGAAGUGGCUGAACCGGGUGGAGACCUACAUCA | |
| | AGUGGCCCUGGUGGGUGUGGCUGUGCAUCAGCGUGGUGCUGAUCU | |
| | UCGUGGUGAGCAUGCUGCUGCUGUGCUGCUGCAGCACCGGCUGCU | |
| | GCGGCUUCUUCAGCUGCUUCGCCAGCAGCAUCCGGGGUUGCUGCG | |
| | AGAGCACCAAGUUACCCUACUACGACGUGGAGAAGAUCCACAUCC | |
| | AG | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MFVLLVAYALLHIAGCQTTNGLNTSYSVCNGCVGYSENVFAVESGGYI | 23 |
| amino acid | PSDFAFNNWFLLTNTSSVVDGVVRSFQPLLLNCLWSVSGLRFTTGFVYF | |
| sequence | NGTGRGDCKGFSSDVLSDVIRYNLNFEENLRRGTILFKTSYGVVVFYCT | |
| | NNTLVSGDAHIPFGTVLGNFYCFVNTTIGNETTSAFVGALPKTVREFVIS | |
| | RTGHFYINGYRYFTLGNVEAVNFNVTTAETTDFCTVALASYADVLVNV | |
| | HKHTFIVLYVDFKPQSGGGKCFNCYPAGVNITLANFNETKGPLCVDTSH | |
| | SQTSIANIIYCNSVINRLRCDQLSFDVPDGFYSTSPIQSVELPVSIVSLPVY | |
| | HKHTFIVLYVDFKPQSGGGKCFNCYPAGVNITLANFNETKGPLCVDTSH | |
| | FTTKYVAVYANVGRWSASINTGNCPFSFGKVNNFVKFGSVCFSLKDIPG | |
| | GCAMPIVANWAYSKYYTIGSLYVSWSDGDGITGVPQPVEGVSSFMNVT | |
| | LDKCTKYNIYDVSGVGVIRVSNDTFLNGITYTSTSGNLLGFKDVTKGTI | |
| | YSITPCNPPDQLVVYQQAVVGAMLSENFTSYGFSNVVELPKFFYASNGT | |
| | YNCTDAVLTYSSFGVCADGSIIAVQPRNVSYDSVSAIVTANLSIPSNWTT | |
| | SVQVEYLQITSTPIVVDCSTYVCNGNVRCVELLKQYTSACKTIEDALRN | |
| | SARLESADVSEMLTFDKKAFTLANVSSFGDYNLSSVIPSLPTSGSRVAGR | |
| | SAIEDILFSKLVTSGLGTVDADYKKCTKGLSIADLACAQYYNGIMVLPG | |
| | VADAERMAMYTGSLIGGIALGGLTSAVSIPFSLAIQARLNYVALQTDVL | |
| | QENQKILAASFNKAMTNIVDAFTGVNDAITQTSQALQTVATALNKIQDV | |
| | VNQQGNSLNHLTSQLRQNFQAISSSIQAIYDRLDPPQADQQVDRLITGRL | |
| | AALNVFVSHTLTKYTEVRASRQLAQQKVNECVKSQSKRYGFCGNGTHI | |
| | FSIVNAAPEGLVFLHTVLLPTQYKDVEAWSGLCVDGTNGYVLRQPNLA | |
| | LYKEGNYYRITSRIMFEPRIPTMADFVQIENCNVTFVNISRSELQTIVPEYI | |
| | DVNKTLQELSYKLPNYTVPDLVVEQYNQTILNLTSEISTLENKSAELNYT | |
| | VQKLQTLIDNINSTLVDLKWLNRVETYIKWPWWVWLCISVVLIFVVSM | |
| | LLLCCCSTGCCGFFSCFASSIRGCCESTKLPYYDVEKIHIQ | |
| PolyA tail | 100 nt | |

NL63_S2P_AY567487

| SEQ ID NO: 24 consists of from 5′ end to 3′ end: 5′ UTR | | 24 |
|---|---|---|
| SEQ ID NO: 2, mRNA ORF SEQ ID NO: 25, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGAAGCUGUUCCUGAUCUUACUGGUGCUGCCCCUGGCCAGCUGC | 25 |
| Construct | UUCUUCACCUGCAACAGCAACGCCAACCUGAGCAUGCUCCAACUG | |
| (excluding the | GGCGUGCCUGACAACAGCUCUACCAUCGUGACCGGCCUGCUGCCC | |
| stop codon) | ACCCACUGGUUCUGCGCCAACCAAAGCACCAGCGUGUACAGCGCA | |
| | AACGGCUUCUUCUACAUCGACGUGGGCAACCACCGGAGCGCCUUC | |
| | GCCCUGCACACCGGCUACUACGACGCCAACCAGUACUAUAUCUAC | |
| | GUGACCAACGAGAUCGGCCUGAACGCCAGCGUGACCCUGAAGAUC | |
| | UGCAAGUUCAGCAGGAACACCACCUUCGACUUCCUGAGCAACGCC | |
| | AGUAGCAGCUUCGACUGCAUUGUGAACCUGCUGUUCACCGAGCAG | |
| | CUGGGAGCCCCUCUGGGCAUCACCAUCAGCGGCGAGACCGUGCGG | |
| | CUGCACCUGUACAACGUGACCCGGACCUUCUACGUGCCCGCCGCCU | |
| | ACAAGCUGACCAAGCUGAGCGUGAAGUGCUACUUCAACUACAGCU | |
| | GCGUGUUUAGCGUGGUAAACGCCACCGUGACCGUGAACGUGACCA | |
| | CCCACAACGGCCGGGUGGUGAACUACACCGUGUGCGACGACUGCA | |
| | ACGGCUACACCGACAACAUCUUUAGCGUGCAGCAGGACGGGCGGA | |
| | UCCCCAACGGCUUUCCCUUCAACAACUGGUUCCUGCUGACCAACG | |
| | GCAGCACCCUGGUGGACGGCGUGAGCCGGCUGUACCAGCCCCUGC | |
| | GGCUGACCUGCCUGUGGCCCGUGCCCGGUCUGAAGAGCUCCACCG | |
| | GCUUCGUGUACUUCAACGCCACUGGCAGCGACGUGAAUUGUAACG | |
| | GCUAUCAGCACAACAGCGUGGUGGACGUGAUGCGGUACAACCUGA | |
| | ACUUCAGCGCCAACAGCCUGGACAACCUUAAGAGCGGCGUGAUCG | |
| | UGUUUAAGACACUGCAGUACGACGUGCUCUUCUACUGCAGCAACA | |
| | GCAGCAGCGGCGUGCUGGACACCACCAUACCCUUCGGCCCCAGCAG | |
| | CCAGCCCUACUAUUGCUUCAUCAACAGCACCAUCAACACCACCCAC | |
| | GUGUCUACCUUCGUGGGCAUCCUGCCGCCCACCGUGCGGGAGAUC | |
| | GUUGUGGCCCGGACCGGCCAGUUCUACAUCAACGGCUUCAAGUAC | |
| | UUCGACCUUGGCUUCAUCGAGGCCGUGAACUUCAACGUGACAACC | |
| | GCCAGCGCCACCGACUUCUGGACCGUGGCCUUCGCCACCUUUGUG | |
| | GACGUGCUGGUCAACGUGAGCGCCACCAACAUCCAGAAUCUGCUG | |
| | UACUGCGACAGCCCCUUCGAGAAGCUGCAGUGCGAGCACCUGCAG | |
| | UUCGGCCUGCAGGACGGCUUCUACAGCGCCAACUUCCUGGACGAC | |
| | AACGUGCUGCCCGAGACCUACGUCGCCCUGCCCAUCUACUACCAGC | |
| | ACACCGACAUCAACUUCACCGCCACCGCCAGCUUCGGAGGCAGCUG | |
| | UUACGUGUGCAAGCCCCACCAGGUGAACAUCAGUCUGAACGGCAA | |
| | CACUAGUGUGUGCGUGCGGACCAGCCACUUCAGCAUACGGUACAU | |
| | CUACAACCGGGUGAAGAGCGGAAGCCCCGGCGACAGCAGCUGGCA | |
| | CAUCUACCUAAAGAGCGGCACCUGCCCCUUCAGCUUUAGCAAGCU | |
| | GAACAACUUCCAGAAGUUCAAGACCAUUUGCUUCAGCACCGUGGA | |
| | GGUGCCCGGCAGCUGUAACUUUCCCCUGGAAGCCACCUGGCACUA | |
| | CACCAGCUACACCAUCGUGGGCGCCCUGUACGUGACCUGGAGCGA | |
| | GGGCAACAGCAUCACCGGCGUGCCCUACCCCGUGAGCGGCAUCCG | |
| | GGAGUUCAGCAACCUGGUGCUGAAUAACUGCACGAAGUACAACAU | |
| | CUACGAUUACGUGGGCACCGGCAUCAUCCGGUCUAGCAACCAGAG | |
| | CCUGGCCGGCGGCAUCACCUACGUAAGCAACAGCGGCAACCUGCU | |
| | GGGCUUCAAGAACGUUAGCACCGGCAACAUCUUCAUCGUGACGCC | |
| | CUGCAACCAGCCCGACCAGGUGGCCGUGUACCAGCAGAGCAUCAU | |
| | CGGCGCCAUGACCGCCGUGAACGAGAGCCGGUACGGUCUGCAGAA | |
| | CCUGCUGCAGCUGCCCAACUUCUACUACGUGAGCAACGGCGGCAA | |
| | CAACUGCACCACCGCUGUGAUGACCUACAGCAACUUCGGCAUCUG | |
| | CGCCGACGGCAGCCUGAUCCCCGUGCGGCCCCGGAACAGCAGCGAU | |
| | AACGGCAUCAGCGCCAUCAUCACCGCCAAUCUGAGCAUCCCCAGCA | |
| | ACUGGACCACCAGCGUGCAGGUGGAGUACCUGCAGAUCACCUCAA | |
| | CUCCCAUCGUGGUGGACUGCGCCACCUACGUGUGUAACGGCAAUC | |
| | CCCGGUGCAAGAACCUGUUGAAGCAGUACACCAGCGCCUGCAAGA | |
| | CCAUCGAGGACGCCCUGAGGCUGAGCGCCCACCUGGAGACCAACG | |
| | ACGUGAGCAGCAUGCUUACCUUCGACUCCAACGCCUUCAGCCUGG | |
| | CCAACGUGACUAGCUUCGGCGACUAUAACCUGAGCUCUGUGCUGC | |
| | CCCAGCGGAACAUCCGGAGCUCCCGGAUCGCCGGCAGAAGCGCCCU | |
| | GGAGGACCUGCUGUUCAGCAAGGUGGUGACCAGCGGCCUGGGCAC | |
| | CGUGGACGUGGAUUACAAGAGCUGCACCAAGGGCCUGAGCAUCGC | |
| | CGAUCUGGCCUGCGCCCAGUACUACAACGGCAUUAUGGUGUUACC | |
| | CGGCGUUGCCGACGCCGAGCGGAUGGCCAUGUACACCGGCAGUCU | |
| | GAUCGGCGGCAUGGUGCUGGGCGGCCUGACAAGCGCCGCCGCCAU | |
| | CCCCUUUAGCCUGGCUCUGCAGGCCCGGCUGAACUACGUGGCCCU | |
| | GCAAACCGACGUGCUGCAGGAGAACCAGAAGAUCCUGGCCGCCUC | |
| | AUUCAACAAGGCCAUCAACAACAUUGUGGCCAGCUUCAGCAGCGU | |
| | GAACGACGCCAUCACCCAGACCGCCGAGGCCAUCCACACCGUCACC | |
| | AUCGCCCUGAACAAGAUCCAGGACGUGGUGAACCAGCAGGGCUCC | |
| | GCCCUUAACCACCUGACCAGCCAGCUGCGGCACAACUUUCAGGCCA | |
| | UCAGCAAUAGCAUCCAGGCCAUCUACGACCGGCUGGACCCACCCCA | |
| | GGCCGACCAGCAGGUGGACCGGCUGAUCACCGGCCGGUUGGCCGC | |
| | ACUGAACGCCUUCGUGAGCCAGGUGCUGAACAAGUACACCGAGGU | |
| | GCGGGGCUCUCGGCGGCUGGCCCAGCAGAAGAUCAACGAGUGCGU | |
| | GAAGAGCCAGAGCAACCGGUACGGCUUCUGCGGCAACGGCACCCA | |
| | CAUCUUCAGCAUCGUGAACAGCGCCCCUGACGGCCUACUGUUCCU | |
| | GCACACCGUGCUGCUGCCCACCGACUACAAGAACGUGAAGGCCUG | |
| | GAGCGGCAUUUGCGUGGACGGCAUCUACGGCUACGUGCUGCGGCA | |
| | GCCCAACUUGGUGCUGUACAGCGACAACGGCGUGUUCCGGGUGAC | |
| | CAGCCGGGUGAUGUUCCAGCCCCGACUGCCCGUUCUGAGCGACUU | |
| | UGUGCAGAUCUACAACUGCAACGUGACCUUCGUGAACAUUAGCCG | |
| | GGUUGAGCUGCAUACCGUGAUCCCCGACUACGUGGACGUUAACAA | |
| | GACCCUGCAGGAGUUCGCCCAGAACUUGCCCAAGUACGUGAAGCC | |
| | CAAUUUCGACCUGACACCCUUCAACCUGACCUACCUGAACCUGAG | |
| | CAGCGAGCUGAAGCAGCUGGAGGCCAAGACCGCCAGCCUGUUCCA | |
| | GACCACCGUGGAGCUGCAGGGCCUGAUCGACCAGAUCAAUAGCAC | |
| | AUACGUGGACCUGAAGCUGCUGAACCGGUUCGAGAACUACAUCAA | |
| | GUGGCCCUGGUGGGUGUGGCUGAUCAUCAGCGUGGUGUUCGUGGU | |
| | GCUGCUGAGCCUGCUGGUGUUCUGCUGCCUGAGCACCGGCUGCUG | |
| | UGGCUGCUGCAACUGCCUGACCUCCAGCAUGCGGGGCUGCUGCGA | |
| | CUGCGGCUCCACCAAGCUGCCCUACUACGAGUUCGAGAAGGUGCA | |
| | CGUGCAG | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MKLFLILLVLPLASCFFTCNSNANLSMLQLGVPDNSSTIVTGLLPTHWFC | 26 |
| amino acid | ANQSTSVYSANGFFYIDVGNHRSAFALHTGYYDANQYYIYVTNEIGLN | |
| sequence | ASVTLKICKFSRNTTFDFLSNASSSFDCIVNLLFTEQLGAPLGITISGETVR | |
| | LHLYNVTRTFYVPAAYKLTKLSVKCYFNYSCVFSVVNATVTVNVTTHN | |
| | GRVVNYTVCDDCNGYTDNIFSVQQDGRIPNGFPFNNWFLLTNGSTLVD | |
| | GVSRLYQPLRLTCLWPVPGLKSSTGFVYFNATGSDVNCNGYQHNSVVD | |
| | VMRYNLNFSANSLDNLKSGVIVFKTLQYDVLFYCSNSSSGVLDTTIPFG | |
| | PSSQPYYCFINSTINTTHVSTFVGILPPTVREIVVARTGQFYINGFKYFDL | |
| | GFIEAVNFNVTTASATDFWTVAFATFVDVLVNVSATNIQNLLYCDSPFE | |
| | KLQCEHLQFGLQDGFYSANFLDDNVLPETYVALPIYYQHTDINFTATAS | |
| | FGGSCYVCKPHQVNISLNGNTSVCVRTSHFSIRYIYNRVKSGSPGDSSW | |
| | HIYLKSGTCPFSFSKLNNFQKFKTICFSTVEVPGSCNFPLEATWHYTSYTI | |
| | VGALYVTWSEGNSITGVPYPVSGIREFSNLVLNNCTKYNIYDYVGTGIIR | |
| | SSNQSLAGGITYVSNSGNLLGFKNVSTGNIFIVTPCNQPDQVAVYQQSII | |
| | GAMTAVNESRYGLQNLLQLPNFYYVSNGGNNCTTAVMTYSNFGICAD | |
| | GSLIPVRPRNSSDNGISAIITANLSIPSNWTTSVQVEYLQITSTPIVVDCAT | |
| | YVCNGNPRCKNLLKQYTSACKTIEDALRLSAHLETNDVSSMLTFDSNA | |
| | FSLANVTSFGDYNLSSVLPQRNIRSSRIAGRSALEDLLFSKVVTSGLGTV | |
| | DVDYKSCTKGLSIADLACAQYYNGIMVLPGVADAERMAMYTGSLIGG | |
| | MVLGGLTSAAAIPFSLALQARLNYVALQTDVLQENQKILAASENKAINN | |
| | IVASFSSVNDAITQTAEAIHTVTIALNKIQDVVNQQGSALNHLTSQLRHN | |
| | FQAISNSIQAIYDRLDPPQADQQVDRLITGRLAALNAFVSQVLNKYTEV | |
| | RGSRRLAQQKINECVKSQSNRYGFCGNGTHIFSIVNSAPDGLLFLHTVLL | |
| | PTDYKNVKAWSGICVDGIYGYVLRQPNLVLYSDNGVFRVTSRVMFQPR | |
| | LPVLSDFVQIYNCNVTFVNISRVELHTVIPDYVDVNKTLQEFAQNLPKY | |
| | VKPNFDLTPFNLTYLNLSSELKQLEAKTASLFQTTVELQGLIDQINSTYV | |
| | DLKLLNRFENYIKWPWWVWLIISVVFVVLLSLLVFCCLSTGCCGCCNCL | |
| | TSSMRGCCDCGSTKLPYYEFEKVHVQ | |
| PolyA tail | 100 nt | |

OC43_SP_2P_1273-TM

| SEQ ID NO: 27 consists of from 5′ end to 3′ end: 5′ UTR | | 27 |
|---|---|---|
| SEQ ID NO: 2, mRNA ORF SEQ ID NO: 28, and 3′ UTR SEQ ID NO: 4. | | |
| Chemistry | 1-methylpseudouridine | |
| Cap | C1 | |
| 5′ UTR | GGGAAAUAAGAGAGAAAAGAAGAGUAAGAAGAAAUAUAAGACCC | 2 |
| | CGGCGCCGCCACC | |
| ORF of mRNA | AUGUUCCUGAUCCUGCUGAUCAGCCUGCCCACCGCCUUCGCCGUG | 28 |
| Construct | AUCGGCGACCUGAAGUGCCCUCUGGAGAGCCGGACCGGCAGCCUG | |
| (excluding the | AACAAUAUCGACACCGGCCCUCCCAGCAUCAGCACCGCCACCGUGG | |
| stop codon) | ACGUGACCAACGGCCUGGGCACCUACUACGUGCUGGACCGGGUGU | |
| | ACCUGAACACCACCCUGUUCCUGAACGGCUAUUACCCCACCAGCGG | |
| | CAGCACCUACCGGAAUAUGGCGCUGAAGGGCACCGACAAGCUGAG | |
| | CACCCUGUGGUUCAAGCCACCCUUCCUGAGCGACUUUAUCAACGG | |
| | CAUCUUUGCCAAGGUGAAGAACACCAAGGUGUUCAAGGACGGCGU | |
| | GAUGUACAGCGAGUUCCCCGCCAUCACUAUCGGUAGCACGUUCGU | |
| | GAACACCAGCUACAGCGUGGUGGUACAGCCCCGGACCAUCAACAG | |
| | CACCCAGGACGGAGUGAACAAGCUGCAGGGCCUGCUGGAGGUGAG | |
| | CGUGUGCCAGUACAACAUGUGCGAGUACCCUCACACCAUCUGCCA | |
| | CCCCAACCUCGGCAACCACUUCAAGGAGCUGUGGCACAUGGAUAC | |
| | CGGCGUGGUGAGCUGCCUGUACAAGCGGAACUUCACCUACGACGU | |
| | CAACGCCACCUACCUGUACUUCCACUUCUACCAGGAAGGAGGCAC | |
| | CUUCUACGCCUACUUCACCGACACCGGAGUGGUGACCAAGUUCCU | |
| | GUUCAACGUGUACCUGGGCAUGGCCCUGAGCCACUACUACGUCAU | |
| | GCCCCUGACUUGUAUCAGCCGGCGGAACAUCGGCUUCACCCUGGA | |
| | GUACUGGGUGACCCCACUGACCAGCCGGCAGUACCUGCUGGCCUU | |
| | CAACCAGGACGGCAUCAUCUUCAACGCCGUGGACUGCAUGAGCGA | |
| | UUUCAUGAGCGAGAUCAAGUGCAAGACCCAGAGCAUCGCGCCACC | |
| | CACCGGCGUGUACGAGCUGAACGGGUACACCGUGCAGCCCAUCGC | |
| | CGACGUGUACCGGCGGAAGCCCGACCUGCCAAACUGCAACAUCGA | |
| | GGCCUGGCUGAACGACAAGAGCGUGCCCUCACCCCUGAACUGGGA | |
| | GCGGAAGACCUUCAGCAACUGCAACUUCAACAUGAGCAGCCUGAU | |
| | GAGCUUCAUCCAGGCUGACAGCUUCACCUGCAACAACAUCGACGC | |
| | CGCCAAGAUCUACGGCAUGUGCUUCAGCUCUAUCACCAUCGACAA | |
| | GUUCGCCAUCCCCAACGGCCGGAAGGUGGACCUGCAACUGGGCAA | |
| | CCUGGGCUACCUGCAGAGCUUCAACUACCGGAUCGACACGACCGC | |
| | CACCAGUUGCCAGCUCUACUACAAUCUGCCCGCCGCCAACGUGAGC | |
| | GUGAGCCGGUUCAACCCCAGCACCUGGAACAAGCGGUUCGGCUUC | |
| | AUCGAAGACAGCGUGUUCAAGCCCCAGCCAGCCGGCGUGUUCACC | |
| | AACCACGACGUGGUGUACGCCCAGCACUGCUUUAAGGCCCCUAAG | |
| | AACUUCUGCCCCUGCAAGAGUAACAGCAGCUUGUGUGUUGGCAGC | |
| | GGCCCCGGCAAGAACAACGGCAUUGGCACCUGCCCCGCUGGCACCA | |
| | ACUACCUGACCUGCCACAACCUGUGCAACCCCGACCCCAUCACCUU | |
| | CACCGGCCCCUACAAGUGUCCCCAGACCAAGAGCCUGGUGGGCAU | |
| | CGGCGAGCACUGCAGCGGCCUGGCCGUGAAGAGCGACUACUGCGG | |
| | CGGCAAUCUGUGCACCUGCCAGCCCCAGGCCUUCCUGGGCUGGAG | |
| | CGCCGACAGCUGCCUGCAGGGCGACAAGUGCAACAUCUUCGCCAA | |
| | CCUGAUACUGCACGACGUGAAUAGCGGCCUGACCUGUAGCACCGA | |
| | CCUGCAGAAGGCCAACACCGACAUCAAGCUCGGCGUGUGCGUUAA | |
| | CUACGACCUGUACGGCAUCAGCGGCCAGGGCAUCUUCGUGGAGGU | |
| | GAACGCUACCUACUACAACAGCUGGCAGAACCUGCUGUACGACAG | |
| | CAACGGCAAUUUGUACGGCUUCCGGGACUACAUCACCAACCGGAC | |
| | CUUCAUGAUCCGGAGCUGCUACAGCGGACGGGUGAGCGCCGCUUU | |
| | CCACGCCAACAGCAGCGAGCCCGCCCUGCUGUUUCGGAACAUCAA | |
| | GUGUAACUACGUGUUCAACAACAGCCUGACCCGGCAGUUACAGCC | |
| | CAUCAACUACUUCGACAGCUACCUGGGCUGCGUCGUGAACGCUUA | |
| | CAACAGCACAGCCAUCAGCGUGCAGACCUGCGACCUGACCGUGGG | |
| | CAGCGGGUACUGCGUGGACUACAGCAAGAACCGGCGGUCUCGGCG | |
| | GGCUAUCACCACCGGCUACCGGUUCACCAAUUUCGAGCCCUUCACC | |
| | GUGAACAGCGUGAACGACAGCCUGGAGCCCGUGGGCGGUCUGUAC | |
| | GAGAUCCAGAUCCCCAGCGACUUCACCAUCGGCAACAUGGAGGAG | |
| | UUCAUCCAGACCAGCAGCCCCAAGGUGACCAUCGACUGCGCCGCCU | |
| | UCGUGUGCGGCGACUACGCCGCCUGUAAGAGCCAGCUGGUGGAGU | |
| | ACGGCAGCUUCUGCGACAACAUCAACGCCAUCCUGACCGAGGUUA | |
| | ACGAGCUGCUGGACACCACCCAGCUGCAGGUGGCCAACAGCCUCA | |
| | UGAACGGCGUGACCCUGAGCAUCAAGCUGAAAGACGGGGUGAACU | |
| | UCAACGUGGACGACAUCAACUUCAGCAGCGUGCUGGGCUGCCUGG | |
| | GCAGCGAGUGCAGCAAGGCCUCCAGCCGGAGCGCCAUCGAGGACC | |
| | UGCUGUUCGACAAGGUGAAACUGAGCGACGUGGGCUUCGUGGCCG | |
| | CCUACAACAACUGCACUGGCGGCGCCGAGAUCCGGGACCUGAUCU | |
| | GCGUGCAAAGCUACAAGGGCAUCAAAGUGCUUCCACCCCUGCUGA | |
| | GCGAGAACCAGAUCAGCGGCUACACCCUGGCUGCCACCAGCGCCA | |
| | GCCUGUUUCCUCCCUGGACAGCAGCUGCCGGCGUGCCCUUCUACCU | |
| | GAACGUGCAGUACCGGAUUAACGGACUGGGCGUGACCAUGGACGU | |
| | GCUGAGCCAGAACCAGAAGCUGAUCGCCAACGCCUUCAACAACGC | |
| | CCUGGACGCCAUCCAGGAGGGCUUCGACGCCACCAACAGCGCUCU | |
| | GGUGAAGAUCCAGGCCGUGGUGAACGCCAACGCAGAGGCCCUGAA | |
| | CAACCUGCUGCAACAGCUGAGCAACCGGUUCGGCGCCAUCAGCAG | |
| | CAGCCUGCAGGAGAUCCUGAGCCGGCUGGAUCCUCCUGAGGCCGA | |
| | GGCCCAGAUCGACCGGCUGAUCAACGGCCGCCUGACCGCCCUGAAC | |
| | GCCUACGUGAGCCAGCAGCUGAGCGACAGCACCCUGGUGAAAUUC | |
| | AGCGCCGCCCAGGCCAUGGAGAAGGUGAACGAGUGCGUGAAGUCA | |
| | CAGAGCAGCCGGAUCAACUUCUGCGGCAACGGAAACCACAUCAUC | |
| | AGCCUGGUGCAGAACGCGCCCUACGGUUUGUACUUCAUCCACUUC | |
| | AGCUACGUGCCCACCAAGUACGUGACCGCAAAGGUGAGCCCCGGC | |
| | CUGUGUAUCGCCGGCGACCGGGGCAUUGCACCCAAGAGCGGCUAC | |
| | UUCGUGAACGUGAACAACACCUGGAUGUACACCGGAUCUGGCUAC | |
| | UACUACCCCGAGCCCAUCACCGAGAACAACGUGGUGGUGAUGAGC | |
| | ACCUGCGCCGUGAACUACACCAAGGCCCCUUACGUGAUGCUGAAU | |
| | ACCAGCAUCCCAAACCUGCCCGACUUCCGGGAGGAGCUGGACCAG | |
| | UGGUUUAAGAACCAAACCAGCGUGGCACCCGACCUGAGCCUGGAC | |
| | UACAUUAACGUGACCUUCCUGGACUUGCAGGUGGAGAUGAACAGG | |
| | CUGCAGGAGGCCAUCAAGGUGCUGAACCAGAGCUACAUCAACCUG | |
| | AAGGACAUCGGCACCUACGAGUACUACGUGAAGUGGCCCUGGUAC | |
| | GUGUGGCUGCUGAUCUGCCUGGCCGGCGUGGCCAUGCUGGUGCUG | |
| | CUGUUCUUCAUCUGCUGCUGCACCGGCUGCGGCACCAGCUGCUUC | |
| | AAGAAGUGCGGCGGCUGCUGCGACGACUACACCGGCUAUCAGGAG | |
| | CUGGUGAUCAAGACCAGCCACGACGAC | |
| 3′ UTR | UGAUAAUAGGCUGGAGCCUCGGUGGCCUAGCUUCUUGCCCCUUGG | 4 |
| | GCCUCCCCCCAGCCCCUCCUCCCCUUCCUGCACCCGUACCCCCGUG | |
| | GUCUUUGAAUAAAGUCUGAGUGGGCGGC | |
| Corresponding | MFLILLISLPTAFAVIGDLKCPLESRTGSLNNIDTGPPSISTATVDVTNGLG | 29 |
| amino acid | TYYVLDRVYLNTTLFLNGYYPTSGSTYRNMALKGTDKLSTLWFKPPFL | |
| sequence | SDFINGIFAKVKNTKVFKDGVMYSEFPAITIGSTFVNTSYSVVVQPRTINS | |
| | TQDGVNKLQGLLEVSVCQYNMCEYPHTICHPNLGNHFKELWHMDTGV | |
| | VSCLYKRNFTYDVNATYLYFHFYQEGGTFYAYFTDTGVVTKFLFNVYL | |
| | GMALSHYYVMPLTCISRRNIGFTLEYWVTPLTSRQYLLAFNQDGIIFNA | |
| | VDCMSDFMSEIKCKTQSIAPPTGVYELNGYTVQPIADVYRRKPDLPNCN | |
| | IEAWLNDKSVPSPLNWERKTFSNCNFNMSSLMSFIQADSFTCNNIDAAK | |
| | IYGMCFSSITIDKFAIPNGRKVDLQLGNLGYLQSFNYRIDTTATSCQLYY | |
| | NLPAANVSVSRFNPSTWNKRFGFIEDSVFKPQPAGVFTNHDVVYAQHC | |
| | FKAPKNFCPCKSNSSLCVGSGPGKNNGIGTCPAGTNYLTCHNLCNPDPI | |
| | TFTGPYKCPQTKSLVGIGEHCSGLAVKSDYCGGNLCTCQPQAFLGWSA | |
| | DSCLQGDKCNIFANLILHDVNSGLTCSTDLQKANTDIKLGVCVNYDLY | |
| | GISGQGIFVEVNATYYNSWQNLLYDSNGNLYGFRDYITNRTFMIRSCYS | |
| | GRVSAAFHANSSEPALLFRNIKCNYVFNNSLTRQLQPINYFDSYLGCVV | |
| | NAYNSTAISVQTCDLTVGSGYCVDYSKNRRSRRAITTGYRFTNFEPFTV | |
| | NSVNDSLEPVGGLYEIQIPSDFTIGNMEEFIQTSSPKVTIDCAAFVCGDYA | |
| | ACKSQLVEYGSFCDNINAILTEVNELLDTTQLQVANSLMNGVTLSIKLK | |
| | DGVNFNVDDINFSSVLGCLGSECSKASSRSAIEDLLFDKVKLSDVGFVA | |
| | AYNNCTGGAEIRDLICVQSYKGIKVLPPLLSENQISGYTLAATSASLFPP | |
| | WTAAAGVPFYLNVQYRINGLGVTMDVLSQNQKLIANAFNNALDAIQE | |
| | GFDATNSALVKIQAVVNANAEALNNLLQQLSNRFGAISSSLQEILSRLDP | |
| | PEAEAQIDRLINGRLTALNAYVSQQLSDSTLVKFSAAQAMEKVNECVK | |
| | SQSSRINFCGNGNHIISLVQNAPYGLYFIHFSYVPTKYVTAKVSPGLCIAG | |
| | DRGIAPKSGYFVNVNNTWMYTGSGYYYPEPITENNVVVMSTCAVNYT | |
| | KAPYVMLNTSIPNLPDFREELDQWFKNQTSVAPDLSLDYINVTFLDLQV | |
| | EMNRLQEAIKVLNQSYINLKDIGTYEYYVKWPWYVWLLICLAGVAML | |
| | VLLFFICCCTGCGTSCFKKCGGCCDDYTGYQEL VIKTSHDD | |
| PolyA tail | 100 nt | |

* It should be understood that any one of the open reading frames and/or corresponding amino acid sequences described in Table 1 may include or exclude the signal sequence. It should also be understood that the signal sequence may be replaced by a different signal sequence, for example, any one of SEQ ID NOs: 38-43.

### EMBODIMENTS

1. A composition comprising a messenger ribonucleic acid (mRNA) comprising an open reading frame (ORF) encoding a human coronavirus membrane bound spike protein, wherein the coronavirus is selected from the group consisting of: NL63, OC43, 229E, HKU1, and bat originating coronaviruses comprising a double proline stabilizing mutation, and a lipid nanoparticle.

2. The composition of embodiment 1, wherein the human coronavirus membrane bound spike protein comprises the amino acid sequence of SEQ ID NO: 5.

3. The composition of embodiment 2, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 3.

4. The composition of embodiment 3, wherein the ORF comprises the nucleotide sequence of SEQ ID NO: 3.

5. The composition of embodiment 1, wherein the human coronavirus membrane bound spike protein comprises the amino acid sequence of SEQ ID NO: 8.

6. The composition of embodiment 4, wherein the ORF comprises a nucleotide sequencing having at least 75%, at least 80%, at least 90%, at least 95%, at least 96%, at least 97%, at least 98%, or at least 99% identity to the nucleotide sequence of SEQ ID NO: 7.

7. The composition of embodiment 6, wherein the ORF comprises the nucleotide sequence of SEQ ID NO: 7.

8. The composition of embodiment 1, wherein the human coronavirus membrane bound spike protein comprises the amino acid sequence of SEQ ID NO: 11.

9. The composition of embodiment 8, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 10.

10. The composition of embodiment 9, wherein the ORF comprises the nucleotide sequence of SEQ ID NO: 10.

11. The composition of embodiment 1, wherein the human coronavirus membrane bound spike protein comprises the amino acid sequence of SEQ ID NO: 14.

12. The composition of embodiment 11, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 13.

13. The composition of embodiment 12, wherein the ORF comprises a nucleotide sequence of SEQ ID NO: 13.

14. The composition of embodiment 1, wherein the human coronavirus membrane bound spike protein comprises the amino acid sequence of SEQ ID NO: 17.

15. The composition of embodiment 14, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 16.

16. The composition of embodiment 15, wherein the ORF comprises a nucleotide sequence of SEQ ID NO: 16.

17. The composition of embodiment 1, wherein the human coronavirus membrane bound spike protein comprises the amino acid sequence of SEQ ID NO: 20.

18. The composition of embodiment 17, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 19.

19. The composition of embodiment 18, wherein the ORF comprises a nucleotide sequence of SEQ ID NO: 19.

20. The composition of embodiment 1, wherein the human coronavirus membrane bound spike protein comprises the amino acid sequence of SEQ ID NO: 23.

21. The composition of embodiment 20, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 22.

22. The composition of embodiment 21, wherein the ORF comprises a nucleotide sequence of SEQ ID NO: 22.

23. The composition of embodiment 1, wherein the human coronavirus membrane bound spike protein comprises the amino acid sequence of SEQ ID NO: 26.

24. The composition of embodiment 23, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 25.

25. The composition of embodiment 24, wherein the ORF comprises a nucleotide sequence of SEQ ID NO: 25.

26. The composition of embodiment 1, wherein the human coronavirus membrane bound spike protein comprises the amino acid sequence of SEQ ID NO: 29.

27. The composition of embodiment 26, wherein the ORF comprises a nucleotide sequence having at least 75%, at least 80%, at least 90%, at least 95%, at least 96%, at least 97%, at least 98%, or at least 99% identity to the nucleotide sequence of SEQ ID NO: 28.

28. The composition of embodiment 27, wherein the ORF comprises a nucleotide sequence of SEQ ID NO: 28.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

The entire contents of International Application Nos. PCT/US2015/02740, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

What is claimed is:

1. A composition comprising:
(a) a first messenger ribonucleic acid (mRNA) comprising an open reading frame (ORF) that encodes a first human coronavirus membrane bound spike protein comprising a prefusion conformation stabilizing mutation;
(b) a second mRNA comprising an ORF that encodes a second human coronavirus membrane bound spike protein comprising a prefusion conformation stabilizing mutation; and
(c) a third mRNA comprising an ORF that encodes a third human coronavirus membrane bound spike protein comprising a prefusion conformation stabilizing mutation, and
wherein the first human coronavirus membrane bound spike protein, the second human coronavirus membrane bound spike protein, and the third human coronavirus membrane bound spike protein are from different human coronaviruses, and
wherein the mRNAs of (a) and (b) and (c) are formulated in a lipid nanoparticle.

2. The composition of claim 1, further comprising:
(d) a fourth mRNA comprising an ORF that encodes a fourth human coronavirus membrane bound spike protein comprising a prefusion conformation stabilizing mutation, and
wherein the fourth human coronavirus membrane bound spike protein is from a different human coronavirus than the first, second, and third human coronavirus spike proteins, and
wherein the mRNA of (d) is formulated in the lipid nanoparticle.

3. The composition of claim 2, further comprising:
(e) a fifth mRNA comprising an ORF that encodes a fifth human coronavirus membrane bound spike protein comprising a prefusion conformation stabilizing mutation, and
wherein the mRNA of (e) is in the lipid nanoparticle.

4. The composition of any one of claims 1-3, wherein each of the human coronavirus membrane bound spike proteins has less than 95% sequence similarity to one another.

5. The composition of any one of claims 1-3, wherein each of the human coronavirus membrane bound spike proteins has less than 90% sequence similarity to one another.

6. The composition of any one of claims 1-3, wherein each of the human coronavirus membrane bound spike proteins has less than 80% sequence similarity to one another.

7. The composition of claim 1, wherein the ratio of the first:second:third mRNA is 1:1:1.

8. The composition of claim 2, wherein the ratio of the first:second:third:fourth mRNA is 1:1:1:1.

9. The composition of claim 3, wherein the ratio of the first:second:third:fourth:fifth mRNA is 1:1:1:1:1.

10. The composition of any one of claims 1-9, wherein the prefusion conformation stabilizing mutation is located within a crown of a helix turn region of each human coronavirus membrane bound spike protein, optionally wherein the prefusion conformation stabilizing mutation comprises a double proline mutation or a quadruple glycine (4G) mutation.

11. The composition of claim 10, wherein the crown of the helix turn region comprises about 12 amino acids in the S2 subunit between the heptad region 1 (HR1) and central helix (CH) or heptad 2 (HR2) regions of the S2 subunit of the coronavirus spike protein.

12. The composition of any one of claims 1-11, wherein the prefusion conformation stabilizing mutation is a substitution relative to a wild-type human coronavirus spike protein.

13. The composition of any one of claims 1-12, wherein the first human coronavirus is selected from the group consisting of: MERS-CoV, SARS-CoV, SARS-CoV-2, NL63, OC43, 229E, and HKU1.

14. The composition of any one of claims 1-13, wherein the second human coronavirus is selected from the group consisting of: MERS-CoV, SARS-CoV, SARS-CoV-2, NL63, OC43, 229E, and HKU1.

15. The composition of any one of claims 1-14, wherein the third human coronavirus is selected from the group consisting of: MERS-CoV, SARS-CoV, SARS-CoV-2, NL63, OC43, 229E, and HKU1.

16. The composition of any one of claims 1-15, wherein the fourth human coronavirus is selected from the group consisting of: MERS-CoV, SARS-CoV, SARS-CoV-2, NL63, OC43, 229E, and HKU1.

17. The composition of any one of claims 1-16, wherein the fifth human coronavirus is selected from the group consisting of: MERS-CoV, SARS-CoV, SARS-CoV-2, NL63, OC43, 229E, and HKU1.

18. The composition of any one of claims 1-12, wherein each of the first, second, third, fourth and fifth human coronaviruses is a SARS-CoV-2.

19. The composition of any one of claims 1-12, wherein each of the first, second, third, fourth and fifth human coronaviruses is a selected from the group consisting of: NL63, OC43, 229E, HKU1, and SARS-CoV-2.

20. The composition of any one of claims 1-19, wherein each mRNA comprises a chemical modification.

21. The composition of claim 20, wherein each mRNA is chemically modified with 1-methyl-pseudouridine, such that each U in the sequence is a 1-methyl-pseudouridine.

22. The composition of any one of claims 1-21, wherein the lipid nanoparticle comprises a PEG-modified lipid, a non-cationic lipid, a sterol, an ionizable amino lipid, or any combination thereof.

23. The composition of claim 22, wherein the lipid nanoparticle comprises 0.5-15 mol % PEG-modified lipid; 5-25 mol % non-cationic lipid; 25-55 mol % sterol; and 20-60 mol % ionizable amino lipid.

24. The composition of claim 23, wherein the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol; and the ionizable amino lipid has the structure of Compound 1:

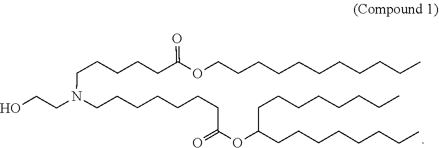 [SMILES: CCCCCCCCCCCOC(=O)CCCCCN(CCO)CCCCCCCC(=O)OC(CCCCCCCC)CCCCCCCC]

25. The composition of any one of claims 1-24, wherein the first human coronavirus is a NL63, the second human coronavirus is a OC43, the third human coronavirus is a 229E, and the fourth human coronavirus is a HKU1.

26. A composition comprising an mRNA vaccine comprising mRNA encoding at least a first and a second human coronavirus antigen, wherein the human coronavirus antigens comprise at least one human coronavirus spike antigen comprising a prefusion conformation stabilizing mutation of a first circulating human coronavirus, and at least one human coronavirus spike antigen comprising a prefusion conformation stabilizing mutation of a second circulating human coronavirus, wherein the second circulating human coronavirus is a later-emerging variant or strain of the first circulating human coronavirus, wherein the first and second human coronavirus spike antigens have at least one amino acid variance from one another, and wherein the composition further comprises a lipid nanoparticle.

27. The composition of claim 26, wherein the human coronavirus antigens are encoded by one or two mRNAs.

28. The composition of claim 27, wherein the mRNA comprises a single mRNA encoding the at least two human coronavirus antigens.

29. The composition of claim 28, wherein the mRNA comprises two mRNA each comprising a single open reading frame (ORF) encoding one of the two human coronavirus antigens.

30. The composition of any one of claims 26-29, wherein the human coronavirus is selected from the group consisting of: NL63, OC43, 229E, and HKU1.

31. The composition of any one of claims 26-30, further comprising:
an mRNA comprising an ORF that encodes a third human coronavirus membrane bound spike protein comprising a prefusion conformation stabilizing mutation, wherein the third human coronavirus membrane bound spike proteins has less than 95% sequence similarity to the first and second membrane bound spike proteins, and wherein the mRNA comprising the ORF that encodes the third human coronavirus membrane bound spike protein is in the lipid nanoparticle.

32. The composition of any one of claims 26-31, further comprising:
an mRNA comprising an ORF that encodes a fourth human coronavirus membrane bound spike protein comprising a prefusion conformation stabilizing mutation, wherein the fourth human coronavirus membrane bound spike proteins has less than 95% sequence similarity to the first and second membrane bound spike proteins, and wherein the mRNA comprising the ORF that encodes the fourth human coronavirus membrane bound spike protein is in the lipid nanoparticle.

33. The composition of any one of claims 26-32, wherein the prefusion conformation stabilizing mutation is located within a crown of a helix turn region of each human coronavirus membrane bound spike protein, optionally wherein the prefusion conformation stabilizing mutation comprises a double proline mutation or a quadruple glycine (4G) mutation.

34. The composition of claim 33, wherein the crown of the helix turn region comprises about 12 amino acids in the S2 subunit between the heptad region 1 (HR1) and central helix (CH) or heptad 2 (HR2) regions of the S2 subunit of the coronavirus spike protein.

35. The composition of any one of claims 26-34, wherein the prefusion conformation stabilizing mutation is a substitution relative to a wild-type human coronavirus spike protein.

36. The composition of any one of claims 26-35, wherein the first, second, third and fourth human coronaviruses are selected from the group consisting of: MERS-CoV, SARS-CoV, SARS-CoV-2, NL63, OC43, 229E, and HKU1.

37. The composition of any one of claims 1-36, wherein the composition is a multivalent mRNA composition and wherein each of the mRNA polynucleotides comprises one or more Identification and Ratio Determination (IDR) sequences.

38. The composition of claim 37, wherein the IDR sequence comprises between 0 and 25 nucleotides.

39. The composition of any one of claims 37-38, wherein the IDR sequence comprises a recognition site for a restriction enzyme.

40. The composition of claim 42, wherein the restriction enzyme is XbaI.

41. The composition of any one of claims 1-40, wherein each of the mRNA polynucleotides in the composition is complementary with and does not interfere with each other mRNA polynucleotide in the composition.

42. A method comprising administering the composition of any one of claims 1-41 to a subject.

43. A method comprising
administering to a subject a composition comprising a lipid nanoparticle comprising:
(a) a first messenger ribonucleic acid (mRNA) comprising an open reading frame (ORF) that encodes a first human coronavirus membrane bound spike protein comprising a prefusion conformation stabilizing mutation, wherein the first human coronavirus membrane bound spike protein is from a first human coronavirus;
(b) a second mRNA comprising an ORF that encodes a second human coronavirus membrane bound spike protein comprising a prefusion conformation stabilizing mutation, wherein the second human coronavirus membrane bound spike protein is from a second human coronavirus;
(c) a third mRNA comprising an ORF that encodes a third human coronavirus membrane bound spike protein comprising a prefusion conformation stabilizing mutation, wherein the third human coronavirus membrane bound spike protein is from a third human coronavirus,
(d) optionally a fourth mRNA comprising an ORF that encodes a fourth human coronavirus membrane bound spike protein comprising a prefusion conformation stabilizing mutation, wherein the fourth human coronavirus membrane bound spike protein is from a fourth human coronavirus; and
(e) optionally a fifth mRNA comprising an ORF that encodes a fifth human coronavirus membrane bound spike protein comprising a prefusion conformation stabilizing mutation, wherein the fifth human coronavirus membrane bound spike protein is from a fifth human coronavirus;
wherein the first human coronavirus, the second human coronavirus, and the third human coronavirus and optionally the fourth human coronavirus and the fifth human coronavirus are from different human coronaviruses, and wherein the composition is in an effective amount for producing a neutralizing antibody response against the first human coronavirus, the second human coronavirus, and the third human coronavirus and optionally the fourth human coronavirus and the fifth human coronavirus.

44. The method of claim 43, wherein the composition is in an effective amount for producing a neutralizing antibody response against a human coronavirus that is within a related clade or is a variant of the first human coronavirus.

45. The method of claim 43 or 44, wherein the composition is in an effective amount for producing a neutralizing antibody response against a human coronavirus that is within a related clade or is a variant of the second human coronavirus.

46. The method of any one of claims 43-45, wherein the composition is in an effective amount for producing a neutralizing antibody response against a human coronavirus that is within a related clade or is a variant of the third human coronavirus.

47. The method of any one of claims 43-46, wherein the composition is in an effective amount for producing a neutralizing antibody response against a human coronavirus that is within a related clade or is a variant of the fourth human coronavirus.

48. The method of any one of claims 43-47, wherein the composition is in an effective amount for producing a neutralizing antibody response against a human coronavirus that is within a related clade or is a variant of the fifth human coronavirus.

49. The method of any one of claims 43-48, wherein each of the human coronavirus membrane bound spike proteins has less than 95% sequence similarity to one another.

50. The method of any one of claims 43-49, wherein the mRNAs of (a) and (b) and (c) and (d) and (e) are in a single lipid nanoparticle composition.

51. The method of any one of claims 43-50, wherein the mRNAs of (a) and (b) and (c) and (d) and (e) are in different lipid nanoparticles.
